SEQUENCE LISTING (38 sequences)

SEQ ID NO: 1 - DNA, 8362 bp, Homo sapiens
  CDS (71)..(8215)
ggctacagtcagtggagaggactttcactgactgactgactgcgtctcaaaacccatggggatccccaccatggatgtgaaagaacgcaggccttactgctccctgaccaagagcagacgagagaaggaacggcgctacacaaattcctccgcagacaatgaggagtgccgggtacccacacagaagtcctacagttccagcgagacattgaaagcttttgatcatgattcctcgcggctgctttacggcaacagagtgaaggatttggttcacagagaagcagacgagttcactagacaaggacagaattttaccctaaggcagttaggagtttgtgaaccagcaactcgaagaggactggcattttgtgcggaaatggggctccctcacagaggttactctatcagtgcagggtcagatgctgatactgaaaatgaagcagtgatgtccccagagcatgccatgagactttggggcaggggggtcaaatcaggccgcagctcctgcctgtcaagtcggtccaactcagccctcaccctgacagatacggagcacgaaaacaagtccgacagtgagaatgagcaacctgcaagcaatcaaggccagtctaccctgcagcccttgccgccttcccataagcagcactctgcacagcatcatccatccatcacttctctcaacagaaactccctgaccaatagaaggaaccagagtccggccccgccggctgctttgcccgccgagctgcaaaccacacccgagtccgtccagctgcaggacagctgggtccttggcagtaatgtaccactggaaagcaggcatttcctattcaaaacaggaacaggtacaacgccactgttcagtactgcaaccccaggatacacaatggcatctggctctgtttattcaccacctactcggccactacctagaaacaccctatcaagaagtgcttttaaattcaagaagtcttcaaagtactgtagctggaaatgcactgcactgtgtgccgtaggggtctcggtgctcctggcaatactcctgtcttattttatagcaatgcatctctttggcctcaactggcagctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaaaggcttaccgccttcccatactcagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcttgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagagggttgtcctggtctgtgcaacagcaatggaagatgtaccctggaccaaaatggctggcattgtgtgtgccagcctggatggagaggagcaggctgtgacgtagccatggagactctttgcacagatagcaaggacaatgaaggagatggactcattgactgcatggaccccgattgctgcctacagagttcctgccagaatcagccctattgtcggggactgccggaccctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatgaagaaagaagagaatgatattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcatcatgggcaatgggcgaaggcgcagcatttcctgccccagttgcaatggtcaagctgatggtaacaagttactggccccagtggcgctagcttgtgggatcgatggcagtctgtacgtaggcgatttcaactatgtgcggcggatattcccttctggaaatgtaacaagtgtcttagaactaagaaataaagattttagacatagcagcaacccagctcatagatactaccttgcaacggacccagtcacgggagatctgtacgtttctgacacaaacacccgcagaatttatcgcccaaagtcacttacgggggcaaaagacttgactaaaaatgcagaagtcgtcgcagggacaggggagcaatgccttccgtttgacgaggcgagatgtggggatggagggaaggccgtggaagccacactcatgagtcccaaaggaatggcagttgataagaatggattaatctactttgttgatggaaccatgattaggaaagttgaccaaaatggaatcatatcaactcttctgggctctaacgatttgacttcagccagacctttaacttgtgacaccagcatgcacatcagccaggtacgtctggaatggcccactgacctagccattaaccctatggataactccatttatgtcctggataataatgtagttttacagatcactgaaaatcgtcaagttcgcattgctgctggacggcccatgcactgtcaggttcccggagtggaatatcctgtggggaagcacgcggtgcagacaacactggaatcagccactgccattgctgtgtcctacagtggggtcctgtacattactgaaactgatgagaagaaaattaaccggataaggcaggtcacaacagatggagaaatctccttagtggccggaataccttcagagtgtgactgcaaaaatgatgccaactgtgactgttaccagagtggagatggctacgccaaggatgccaaactcagtgccccatcctccctggctgcttctccagatggtacactgtatattgcagatctagggaatatccggatacgggctgtgtcaaagaataagcctttacttaactctatgaacttctatgaagttgcgtctccaactgatcaagaactctacatctttgacatcaatggtactcaccaatatactgtaagtttagtcactggtgattacctttacaattttagctacagcaatgacaatgatattactgctgtgacagacagcaatggcaacacccttagaattagacgggacccaaatcgcatgccagttcgagtggtgtctcctgataaccaagtgatatggttgacaataggaacaaatggatgtttgaaaagcatgactgctcaaggactggaattagttttgtttacttaccatggcaatagtggccttttagccactaaaagtgatgaaactggatggacaacgttttttgactatgacagtgaaggtcgtctgacaaatgttacgtttccaactggagtggtcacaaacctgcatggggacatggacaaggctatcacagtggacattgagtcatctagccgagaagaagatgtcagcatcacttcaaatctgtcctcgatcgattctttctacaccatggttcaagatcagttaagaaacagctaccagattggttatgacggctccctcagaattatctacgccagtggcctggactcacactaccaaacagagccgcacgttctggctggcaccgctaatccgacggttgccaaaagaaacatgactttgcctggcgagaacggtcaaaacttggtggaatggagattccgaaaagagcaagcccaagggaaagtcaatgtctttggccgcaagctcagggttaatggcagaaacctcctttcagttgactttgatcgaacaacaaagacagaaaagatctatgacgaccaccgtaaatttctactgaggatcgcctacgacacgtctgggcacccgactctctggctgccaagcagcaagctgatggccgtcaatgtcacctattcatccacaggtcaaattgccagcatccagcgaggcaccactagcgagaaagtagattatgacggacaggggaggatcgtgtctcgggtctttgctgatggtaaaacatggagttacacatatttagaaaagtccatggttcttctgcttcatagccagcggcagtacatcttcgaatacgatatgtgggaccgcctgtctgccatcaccatgcccagtgtggctcgccacaccatgcagaccatccgatccattggctactaccgcaacatatacaaccccccggaaagcaacgcctccatcatcacggactacaacgaggaagggctgcttctacaaacagctttcttgggtacaagtcggagggtcttattcaaatacagaaggcagactaggctctcagaaattttatatgatagcacaagagtcagttttacctatgatgaaacagcaggagtcctaaagacagtaaacctccagagtgatggttttatttgcaccattagatacaggcaaattggtcccctgattgacaggcagattttccgctttagtgaagatgggatggtaaatgcaagatttgactatagctatgacaacagctttcgagtgaccagcatgcagggtgtgatcaatgaaacgccactgcctattgatctgtatcagtttgatgacatttctggcaaagttgagcagtttggaaagtttggagttatatattatgatattaaccagatcatttctacagctgtaatgacctatacgaagcactttgatgctcatggccgtatcaaggagattcaatatgagatattcaggtcgctcatgtactggattacaattcagtatgataacatgggtcgggtaaccaagagagagattaaaatagggccctttgccaacaccaccaaatatgcttatgaatatgatgttgatggacagctccaaacagtttacctcaatgaaaagataatgtggcggtacaactacgatctgaatggaaacctccatttactgaacccaagtaacagtgcgcgtctgacaccccttcgctatgacctgcgagacagaatcactcgactgggtgatgttcaatatcggttggatgaagatggtttcctacgtcaaaggggcacggaaatctttgaatatagctccaaggggcttctaactcgcgtttacagtaaaggcagtggctggacagtgatctaccgttatgacggcctgggaaggcgtgtttctagcaaaaccagtctaggacagcacctgcagtttttttatgctgacttaacttatcccactaggattactcatgtctacaaccattcgagttcagaaattacctccctgtattatgatctccaaggacatctttttgccatggaaatcagcagtggggatgaattctatattgcatcggataacacagggacaccactggctgtgttcagtagcaatgggcttatgctgaaacagattcagtacactgcatatggggaaatctattttgactctaatattgactttcaactggtaattggatttcatggtggcctgtatgacccactcaccaaattaatccactttggagaaagagattatgacattttggcaggacggtggacaacacctgacatagaaatctggaaaagaattgggaaggacccagctccttttaacttgtacatgtttaggaataacaaccctgcaagcaaaatccatgacgtgaaagattacatcacagatgttaacagctggctggtgacatttggtttccatctgcacaatgctattcctggattccctgttcccaaatttgatttaacagaaccttcttacgaacttgtgaagagtcagcagtgggatgatataccgcccatcttcggagtccagcagcaagtggcgcggcaggccaaggccttcctgtcgctggggaagatggccgaggtgcaggtgagccggcgccgggccggcggcgcgcagtcctggctgtggttcgccacggtcaagtcgctgatcggcaagggcgtcatgctggccgtcagccagggccgcgtgcagaccaacgtgctcaacatcgccaacgaggactgcatcaaggtggcggccgtgctcaacaacgccttctacctggagaacctgcacttcaccatcgagggcaaggacacgcactacttcatcaagaccaccacgcccgagagcgacctgggcacgctgcggttgaccagcggccgcaaggcgctggagaacggcatcaacgtgacggtgtcgcagtccaccacggtggtgaacggcaggacgcgcaggttcgcggacgtggagatgcagttcggcgcgctggcgctgcacgtgcgctacggcatgaccctggacgaggagaaggcgcgcatcctggagcaggcgcggcagcgcgcgctcgcccgggcctgggcgcgcgagcagcagcgcgtgcgcgacggcgaggagggcgcgcgcctctggacggagggcgagaagcggcagctgctgagcgccggcaaggtgcagggctacgacgggtactacgtactctcggtggagcagtaccccgagctggccgacagcgccaacaacatccagttcctgcggcagagcgagatcggcaggaggggtaagcctatccctaaccctctcctcggtctcgattctacgcgtaccggtcatcatcaccatcaccattaactcgagctcgagtggtcagtcttcactgactgactgactggaaagaggaagggctggaagaggaaggagcttggc

SEQ ID NO: 2 - protein, 2715 aa, Homo sapiens
MDVKERRPYCSLTKSRREKERRYTNSSADNEECRVPTQKSYSSSETLKAFDHDSSRLLYGNRVKDLVHREADEFTRQGQNFTLRQLGVCEPATRRGLAFCAEMGLPHRGYSISAGSDADTENEAVMSPEHAMRLWGRGVKSGRSSCLSSRSNSALTLTDTEHENKSDSENEQPASNQGQSTLQPLPPSHKQHSAQHHPSITSLNRNSLTNRRNQSPAPPAALPAELQTTPESVQLQDSWVLGSNVPLESRHFLFKTGTGTTPLFSTATPGYTMASGSVYSPPTRPLPRNTLSRSAFKFKKSSKYCSWKCTALCAVGVSVLLAILLSYFIAMHLFGLNWQLQQTENDTFENGKVNSDTMPTNTVSLPSGDNGKLGGFTQENNTIDSGELDIGRRAIQEIPPGIFWRSQLFIDQPQFLKFNISLQKDALIGVYGRKGLPPSHTQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKEGCPGLCNSNGRCTLDQNGWHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMKKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELRNKDFRHSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLSAPSSLAASPDGTLYIADLGNIRIRAVSKNKPLLNSMNFYEVASPTDQELYIFDINGTHQYTVSLVTGDYLYNFSYSNDNDITAVTDSNGNTLRIRRDPNRMPVRVVSPDNQVIWLTIGTNGCLKSMTAQGLELVLFTYHGNSGLLATKSDETGWTTFFDYDSEGRLTNVTFPTGVVTNLHGDMDKAITVDIESSSREEDVSITSNLSSIDSFYTMVQDQLRNSYQIGYDGSLRIIYASGLDSHYQTEPHVLAGTANPTVAKRNMTLPGENGQNLVEWRFRKEQAQGKVNVFGRKLRVNGRNLLSVDFDRTTKTEKIYDDHRKFLLRIAYDTSGHPTLWLPSSKLMAVNVTYSSTGQIASIQRGTTSEKVDYDGQGRIVSRVFADGKTWSYTYLEKSMVLLLHSQRQYIFEYDMWDRLSAITMPSVARHTMQTIRSIGYYRNIYNPPESNASIITDYNEEGLLLQTAFLGTSRRVLFKYRRQTRLSEILYDSTRVSFTYDETAGVLKTVNLQSDGFICTIRYRQIGPLIDRQIFRFSEDGMVNARFDYSYDNSFRVTSMQGVINETPLPIDLYQFDDISGKVEQFGKFGVIYYDINQIISTAVMTYTKHFDAHGRIKEIQYEIFRSLMYWITIQYDNMGRVTKREIKIGPFANTTKYAYEYDVDGQLQTVYLNEKIMWRYNYDLNGNLHLLNPSNSARLTPLRYDLRDRITRLGDVQYRLDEDGFLRQRGTEIFEYSSKGLLTRVYSKGSGWTVIYRYDGLGRRVSSKTSLGQHLQFFYADLTYPTRITHVYNHSSSEITSLYYDLQGHLFAMEISSGDEFYIASDNTGTPLAVFSSNGLMLKQIQYTAYGEIYFDSNIDFQLVIGFHGGLYDPLTKLIHFGERDYDILAGRWTTPDIEIWKRIGKDPAPFNLYMFRNNNPASKIHDVKDYITDVNSWLVTFGFHLHNAIPGFPVPKFDLTEPSYELVKSQQWDDIPPIFGVQQQVARQAKAFLSLGKMAEVQVSRRRAGGAQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGRR

SEQ ID NO: 3 - DNA, 7786 bp, Homo sapiens
  CDS (476)..(7603)
aacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagctctagccaccatggagacagacacactcctgctatgggtactgctgctctgggttccaggttccactggtgacgcggcccagccggccaggcgcgcgcgccgtacgaagctttcgcgaggatccctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaaaggcttaccgccttcccatactcagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcttgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagagggttgtcctggtctgtgcaacagcaatggaagatgtaccctggaccaaaatggctggcattgtgtgtgccagcctggatggagaggagcaggctgtgacgtagccatggagactctttgcacagatagcaaggacaatgaaggagatggactcattgactgcatggaccccgattgctgcctacagagttcctgccagaatcagccctattgtcggggactgccggaccctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatgaagaaagaagagaatgatattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcatcatgggcaatgggcgaaggcgcagcatttcctgccccagttgcaatggtcaagctgatggtaacaagttactggccccagtggcgctagcttgtgggatcgatggcagtctgtacgtaggcgatttcaactatgtgcggcggatattcccttctggaaatgtaacaagtgtcttagaactaagaaataaagattttagacatagcagcaacccagctcatagatactaccttgcaacggacccagtcacgggagatctgtacgtttctgacacaaacacccgcagaatttatcgcccaaagtcacttacgggggcaaaagacttgactaaaaatgcagaagtcgtcgcagggacaggggagcaatgccttccgtttgacgaggcgagatgtggggatggagggaaggccgtggaagccacactcatgagtcccaaaggaatggcagttgataagaatggattaatctactttgttgatggaaccatgattaggaaagttgaccaaaatggaatcatatcaactcttctgggctctaacgatttgacttcagccagacctttaacttgtgacaccagcatgcacatcagccaggtacgtctggaatggcccactgacctagccattaaccctatggataactccatttatgtcctggataataatgtagttttacagatcactgaaaatcgtcaagttcgcattgctgctggacggcccatgcactgtcaggttcccggagtggaatatcctgtggggaagcacgcggtgcagacaacactggaatcagccactgccattgctgtgtcctacagtggggtcctgtacattactgaaactgatgagaagaaaattaaccggataaggcaggtcacaacagatggagaaatctccttagtggccggaataccttcagagtgtgactgcaaaaatgatgccaactgtgactgttaccagagtggagatggctacgccaaggatgccaaactcagtgccccatcctccctggctgcttctccagatggtacactgtatattgcagatctagggaatatccggatacgggctgtgtcaaagaataagcctttacttaactctatgaacttctatgaagttgcgtctccaactgatcaagaactctacatctttgacatcaatggtactcaccaatatactgtaagtttagtcactggtgattacctttacaattttagctacagcaatgacaatgatattactgctgtgacagacagcaatggcaacacccttagaattagacgggacccaaatcgcatgccagttcgagtggtgtctcctgataaccaagtgatatggttgacaataggaacaaatggatgtttgaaaagcatgactgctcaaggactggaattagttttgtttacttaccatggcaatagtggccttttagccactaaaagtgatgaaactggatggacaacgttttttgactatgacagtgaaggtcgtctgacaaatgttacgtttccaactggagtggtcacaaacctgcatggggacatggacaaggctatcacagtggacattgagtcatctagccgagaagaagatgtcagcatcacttcaaatctgtcctcgatcgattctttctacaccatggttcaagatcagttaagaaacagctaccagattggttatgacggctccctcagaattatctacgccagtggcctggactcacactaccaaacagagccgcacgttctggctggcaccgctaatccgacggttgccaaaagaaacatgactttgcctggcgagaacggtcaaaacttggtggaatggagattccgaaaagagcaagcccaagggaaagtcaatgtctttggccgcaagctcagggttaatggcagaaacctcctttcagttgactttgatcgaacaacaaagacagaaaagatctatgacgaccaccgtaaatttctactgaggatcgcctacgacacgtctgggcacccgactctctggctgccaagcagcaagctgatggccgtcaatgtcacctattcatccacaggtcaaattgccagcatccagcgaggcaccactagcgagaaagtagattatgacggacaggggaggatcgtgtctcgggtctttgctgatggtaaaacatggagttacacatatttagaaaagtccatggttcttctgcttcatagccagcggcagtacatcttcgaatacgatatgtgggaccgcctgtctgccatcaccatgcccagtgtggctcgccacaccatgcagaccatccgatccattggctactaccgcaacatatacaaccccccggaaagcaacgcctccatcatcacggactacaacgaggaagggctgcttctacaaacagctttcttgggtacaagtcggagggtcttattcaaatacagaaggcagactaggctctcagaaattttatatgatagcacaagagtcagttttacctatgatgaaacagcaggagtcctaaagacagtaaacctccagagtgatggttttatttgcaccattagatacaggcaaattggtcccctgattgacaggcagattttccgctttagtgaagatgggatggtaaatgcaagatttgactatagctatgacaacagctttcgagtgaccagcatgcagggtgtgatcaatgaaacgccactgcctattgatctgtatcagtttgatgacatttctggcaaagttgagcagtttggaaagtttggagttatatattatgatattaaccagatcatttctacagctgtaatgacctatacgaagcactttgatgctcatggccgtatcaaggagattcaatatgagatattcaggtcgctcatgtactggattacaattcagtatgataacatgggtcgggtaaccaagagagagattaaaatagggccctttgccaacaccaccaaatatgcttatgaatatgatgttgatggacagctccaaacagtttacctcaatgaaaagataatgtggcggtacaactacgatctgaatggaaacctccatttactgaacccaagtaacagtgcgcgtctgacaccccttcgctatgacctgcgagacagaatcactcgactgggtgatgttcaatatcggttggatgaagatggtttcctacgtcaaaggggcacggaaatctttgaatatagctccaaggggcttctaactcgcgtttacagtaaaggcagtggctggacagtgatctaccgttatgacggcctgggaaggcgtgtttctagcaaaaccagtctaggacagcacctgcagtttttttatgctgacttaacttatcccactaggattactcatgtctacaaccattcgagttcagaaattacctccctgtattatgatctccaaggacatctttttgccatggaaatcagcagtggggatgaattctatattgcatcggataacacagggacaccactggctgtgttcagtagcaatgggcttatgctgaaacagattcagtacactgcatatggggaaatctattttgactctaatattgactttcaactggtaattggatttcatggtggcctgtatgacccactcaccaaattaatccactttggagaaagagattatgacattttggcaggacggtggacaacacctgacatagaaatctggaaaagaattgggaaggacccagctccttttaacttgtacatgtttaggaataacaaccctgcaagcaaaatccatgacgtgaaagattacatcacagatgttaacagctggctggtgacatttggtttccatctgcacaatgctattcctggattccctgttcccaaatttgatttaacagaaccttcttacgaacttgtgaagagtcagcagtgggatgatataccgcccatcttcggagtccagcagcaagtggcgcggcaggccaaggccttcctgtcgctggggaagatggccgaggtgcaggtgagccggcgccgggccggcggcgcgcagtcctggctgtggttcgccacggtcaagtcgctgatcggcaagggcgtcatgctggccgtcagccagggccgcgtgcagaccaacgtgctcaacatcgccaacgaggactgcatcaaggtggcggccgtgctcaacaacgccttctacctggagaacctgcacttcaccatcgagggcaaggacacgcactacttcatcaagaccaccacgcccgagagcgacctgggcacgctgcggttgaccagcggccgcaaggcgctggagaacggcatcaacgtgacggtgtcgcagtccaccacggtggtgaacggcaggacgcgcaggttcgcggacgtggagatgcagttcggcgcgctggcgctgcacgtgcgctacggcatgaccctggacgaggagaaggcgcgcatcctggagcaggcgcggcagcgcgcgctcgcccgggcctgggcgcgcgagcagcagcgcgtgcgcgacggcgaggagggcgcgcgcctctggacggagggcgagaagcggcagctgctgagcgccggcaaggtgcagggctacgacgggtactacgtactctcggtggagcagtaccccgagctggccgacagcgccaacaacatccagttcctgcggcagagcgagatcggcaggaggggtaagcctatccctaaccctctcctcggtctcgattctacgcgtaccggtcatcatcaccatcaccattaactcgagggtaagcctatccctaaccctctcctcggtctcgattctacgcgtaccggtcatcaccaccatcaccattgagtttaattcatgatcatatcagccatacacatt

SEQ ID NO: 4 - protein, 2376 aa, Homo sapiens
LQQTENDTFENGKVNSDTMPTNTVSLPSGDNGKLGGFTQENNTIDSGELDIGRRAIQEIPPGIFWRSQLFIDQPQFLKFNISLQKDALIGVYGRKGLPPSHTQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKEGCPGLCNSNGRCTLDQNGWHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMKKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELRNKDFRHSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLSAPSSLAASPDGTLYIADLGNIRIRAVSKNKPLLNSMNFYEVASPTDQELYIFDINGTHQYTVSLVTGDYLYNFSYSNDNDITAVTDSNGNTLRIRRDPNRMPVRVVSPDNQVIWLTIGTNGCLKSMTAQGLELVLFTYHGNSGLLATKSDETGWTTFFDYDSEGRLTNVTFPTGVVTNLHGDMDKAITVDIESSSREEDVSITSNLSSIDSFYTMVQDQLRNSYQIGYDGSLRIIYASGLDSHYQTEPHVLAGTANPTVAKRNMTLPGENGQNLVEWRFRKEQAQGKVNVFGRKLRVNGRNLLSVDFDRTTKTEKIYDDHRKFLLRIAYDTSGHPTLWLPSSKLMAVNVTYSSTGQIASIQRGTTSEKVDYDGQGRIVSRVFADGKTWSYTYLEKSMVLLLHSQRQYIFEYDMWDRLSAITMPSVARHTMQTIRSIGYYRNIYNPPESNASIITDYNEEGLLLQTAFLGTSRRVLFKYRRQTRLSEILYDSTRVSFTYDETAGVLKTVNLQSDGFICTIRYRQIGPLIDRQIFRFSEDGMVNARFDYSYDNSFRVTSMQGVINETPLPIDLYQFDDISGKVEQFGKFGVIYYDINQIISTAVMTYTKHFDAHGRIKEIQYEIFRSLMYWITIQYDNMGRVTKREIKIGPFANTTKYAYEYDVDGQLQTVYLNEKIMWRYNYDLNGNLHLLNPSNSARLTPLRYDLRDRITRLGDVQYRLDEDGFLRQRGTEIFEYSSKGLLTRVYSKGSGWTVIYRYDGLGRRVSSKTSLGQHLQFFYADLTYPTRITHVYNHSSSEITSLYYDLQGHLFAMEISSGDEFYIASDNTGTPLAVFSSNGLMLKQIQYTAYGEIYFDSNIDFQLVIGFHGGLYDPLTKLIHFGERDYDILAGRWTTPDIEIWKRIGKDPAPFNLYMFRNNNPASKIHDVKDYITDVNSWLVTFGFHLHNAIPGFPVPKFDLTEPSYELVKSQQWDDIPPIFGVQQQVARQAKAFLSLGKMAEVQVSRRRAGGAQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGRR

SEQ ID NO: 5 - DNA, 2482 bp, Homo sapiens
  CDS (11)..(2473)
cacctcgcgaaactggcagctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaaaggcttaccgccttcccatactcagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcttgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagagggttgtcctggtctgtgcaacagcaatggaagatgtaccctggaccaaaatggctggcattgtgtgtgccagcctggatggagaggagcaggctgtgacgtagccatggagactctttgcacagatagtaaggacaatgaaggagatggactcattgactgcatggatcccgattgctgcctacagagttcctgccagaatcagccctattgtcggggactgccggatcctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatgaagaaagaagagaatgacattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcctcgagggc

SEQ ID NO: 6 - protein, 821 aa, Homo sapiens
NWQLQQTENDTFENGKVNSDTMPTNTVSLPSGDNGKLGGFTQENNTIDSGELDIGRRAIQEIPPGIFWRSQLFIDQPQFLKFNISLQKDALIGVYGRKGLPPSHTQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKEGCPGLCNSNGRCTLDQNGWHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMKKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSS

SEQ ID NO: 7 - DNA, 8657 bp, Homo sapiens
  CDS (151)..(8325)
tttggcctcgggccagaattcggcacgaggggtctggagcttggaggagaagtctgaactaaggataaactaaagagaggccaatgagacttgaaccctgagcctaagttgtcaccagcaggactgatgtgcacacagaaggaatgaagtatggatgtgaaagaacgcaggccttactgctccctgaccaagagcagacgagagaaggaacggcgctacacaaattcctccgcagacaatgaggagtgccgggtacccacacagaagtcctacagttccagcgagacattgaaagcttttgatcatgattcctcgcggctgctttacggcaacagagtgaaggatttggttcacagagaagcagacgagttcactagacaaggacagaattttaccctaaggcagttaggagtttgtgaaccagcaactcgaagaggactggcattttgtgcggaaatggggctccctcacagaggttactctatcagtgcagggtcagatgctgatactgaaaatgaagcagtgatgtccccagagcatgccatgagactttggggcaggggggtcaaatcaggccgcagctcctgcctgtcaagtcggtccaactcagccctcaccctgacagatacggagcacgaaaacaagtccgacagtgagaatgagcaacctgcaagcaatcaaggccagtctaccctgcagcccttgccgccttcccataagcagcactctgcacagcatcatccatccatcacttctctcaacagaaactccctgaccaatagaaggaaccagagtccggccccgccggctgctttgcccgccgagctgcaaaccacacccgagtccgtccagctgcaggacagctgggtccttggcagtaatgtaccactggaaagcaggcatttcctattcaaaacaggaacaggtacaacgccactgttcagtactgcaaccccaggatacacaatggcatctggctctgtttattcaccacctactcggccactacctagaaacaccctatcaagaagtgcttttaaattcaagaagtcttcaaagtactgtagctggaaatgcactgcactgtgtgccgtaggggtctcggtgctcctggcaatactcctgtcttattttatagcaatgcatctctttggcctcaactggcagctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaagaagttaccgccttcccatactcagtcctccccccagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcctgtgcttgcagctcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagacaagataggatataaagagggttgtcctggtctgtgcaacagcaatggaagatgtaccctggaccaaaatggcggacattgtgtgtgccagcctggatggagaggagcaggctgtgacgtagccatggagactctttgcacagatagcaaggacaatgaaggggatggactcattgactgcatggatcccgattgctgcctacagagttcctgccagaatcagccctattgtcggggactgccggatcctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatggagaaagaagagaatgacattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcatcatgggcaatgggcgaaggcgcagcatttcctgccccagttgcaatggtcaagctgatggtaacaagttactggccccagtggcgctagcttgtgggatcgatggcagtctgtacgtaggcgatttcaactacgtgcggcggatattcccttctggaaatgtaacaagtgtcttagaactaagaaataaagattttagacatagcagcaacccagctcatagatactaccttgcaacggatccagtcacgggagatctgtacgtttctgacacaaacacccgcagaatttatcgcccaaagtcacttacgggggcaaaagacttgactaaaaatgcagaagtcgtcgcagggacaggggagcaatgccttccgtttgacgaggcgagatgtggggatggagggaaggccgtggaagccacactcatgagtcccaaaggaatggcagttgataagaatggattaatctactttgttgatggaaccatgattaggaaagttgaccaaaatggaatcatatcaactcttctgggctctaacgatttgacttcagccagacctttaacttgtgacaccagcatgcacatcagccaggtacgtctggaatggcccactgacctagccattaaccctatggataactccatttatgtcctggataataatgtagttttacagatcactgaaaatcgtcaagttcgcattgctgctggacggcccatgcactgtcaggttcccggagtggaatatcctgtggggaagcacgcggtgcagacaacactggaatcagccactgccattgctgtgtcctacagtggggtcctgtacattactgaaactgatgagaagaaaattaaccggataaggcaggtcacaacagatggagaaatctccttagtggccggaataccttcagagtgtgactgcaaaaatgatgccaactgtgactgttaccagagtggagatggctacgccaaggatgccaaactcagtgccccatcctccctggctgcttctccagatggtacactgtatattgcagatctagggaatatccggatccgggctgtgtcaaagaataagcctttacttaactctatgaacttctatgaagttgcgtctccaactgatcaagaactctacatctttgacatcaatggtactcaccaatatactgtaagtttagtcactggtgattacctttacaattttagctacagcaatgacaatgatattactgctgtgacagacagcaatggcaacacccttagaattagacgggacccaaatcgcatgccagttcgagtggtgtctcctgataaccaagtgatatggttgacaataggaacaaatggatgtttgaaaggcatgactgctcaaggactggaattagttttgtttacttaccatggcaatagtggccttttagccactaaaagtgatgaaactggatggacaacgttttttgactatgacagtgaaggtcgtctgacaaatgttacgtttccaactggagtggtcacaaacctgcatggggacatggacaaggctatcacagtggacattgagtcatctagccgagaagaagatgtcagcatcacttcaaatctgtcctcgatcgattctttctacaccatggttcaagatcagttaagaaacagctaccagattggttatgacggctccctcagaattatctacgccagtggcctggactcacactaccaaacagagccgcacgttctggctggcaccgctaatccgacggttgccaaaagaaacatgactttgcctggcgagaacggtcaaaacttggtggaatggagattccgaaaagagcaagcccaagggaaagtcaatgtctttggccgcaagctcagggttaatggcagaaacctcctttcagttgactttgatcgaacaacaaagacagaaaagatctatgacgaccaccgtaaatttctactgaggatcgcctacgacacgtctgggcacccgactctctggctgccaagcagcaagctgatggccgtcaatgtcacctattcatccacaggtcaaattgccagcatccagcgaggcaccactagcgagaaagtagattatgacggacaggggaggatcgtgtctcgggtctttgctgatggtaaaacatggagttacacatatttagaaaagtccatggttcttctgcttcatagccagcggcagtacatcttcgaatacgatatgtgggaccgcctgtctgccatcaccatgcccagtgtggctcgccacaccatgcagaccatccgatccattggctactaccgcaacatatacaaccccccggaaagcaacgcctccatcatcacggactacaacgaggaagggctgcttctacaaacagctttcttgggtacaagtcggagggtcttattcaaatacagaaggcagactaggctctcagaaattttatatgatagcacaagagtcagttttacctatgatgaaacagcaggagtcctaaagacagtaaacctccagagtgatggttttatttgcaccattagatacaggcaaattggtcccctgattgacaggcagattttccgctttagtgaagatgggatggtaaatgcaagatttgactatagctatgacaacagctttcgagtgaccagcatgcagggtgtgatcaatgaaacgccactgcctattgatctgtatcagtttgatgacatttctggcaaagttgagcagtttggaaagtttggagttatatattatgatattaaccagatcatttctacagctgtaatgacctatacgaagcactttgatgctcatggccgtatcaaggagattcaatatgagatattcaggtcgctcatgtactggattacaattcagtatgataacatgggtcgggtaaccaagagagagattaaaatagggccctttgccaacaccaccaaatatgcttatgaatatgatgttgatggacagctccaaacagtttacctcaatgaaaagataatgtggcggtacaactacgatctgaatggaaacctccatttactgaacccaagtaacagtgcgcgtctgacaccccttcgctatgacctgcgagacagaatcactcgactgggtgatgttcaatatcggttggatgaagatggtttcctacgtcaaaggggcacggaaatctttgaatatagctccaaggggcttctaactcgagtttacagtaaaggcagtggctggacagtgatctaccgttatgacggcctgggaaggcgtgtttctagcaaaaccagtctaggacagcacctgcagtttttttatgctgacttaacttatcccactaggattactcatgtctacaaccattcgagttcagaaattacctccctgtattatgatctccaaggacatctttttgccatggaaatcagcagtggggatgaattctatattgcatcggataacacagggacaccactggctgtgttcagtagcaatgggcttatgctgaaacagattcagtacactgcatatggggaaatctattttgactctaatattgactttcaactggtaattggatttcatggtggcctgtatgacccactcaccaaattaatccactttggagaaagagattatgacattttggcaggacggtggacaacacctgacatagaaatctggaaaagaattgggaaggacccagctccttttaacttgtacatgtttaggaataacaaccctgcaagcaaaatccatgacgtgaaagattacatcacagatgttaacagctggctggtgacatttggtttccatctgcacaatgctattcctggattccctgttcccaaatttgatttaacagaaccttcttacgaacttgtgaagagtcagcagtgggatgatataccgcccatcttcggagtccagcagcaagtggcgcggcaggccaaggccttcctgtcgctggggaagatggccgaggtgcaggtgagccggcgccgggccggcggcgcgcagtcctggctgtggttcgccacggtcaagtcgctgatcggcaagggcgtcatgctggccgtcagccagggccgcgtgcagaccaacgtgctcaacatcgccaacgaggactgcatcaaggtggcggccgtgctcaacaacgccttctacctggagaacctgcacttcaccatcgagggcaaggacacgcactacttcatcaagaccaccacgcccgagagcgacctgggcacgctgcggttgaccagcggccgcaaggcgctggagaacggcatcaacgtgacggtgtcgcagtccaccacggtggtgaacggcaggacgcgcaggttcgcggacgtggagatgcagttcggcgcgctggcgctgcacgtgcgctacggcatgaccctggacgaggagaaggcgcgcatcctggagcaggcgcggcagcgcgcgctcgcccgggcctgggcgcgcgagcagcagcgcgtgcgcgacggcgaggagggcgcgcgcctctggacggagggcgagaagcggcagctgctgagcgccggcaaggtgcagggctacgacgggtactacgtactctcggtggagcagtaccccgagctggccgacagcgccaacaacatccagttcctgcggcagagcgagatcggcaggaggtaacgcccgggccgcgcccgccgagccgctcacgccctgcccacattgtcctgtggcacaacccgagtgggactctccaacgcccaagagccttcctcccgggggaatgagactgctgttacgacccacacccacaccgcgaaaacaaggaccgcttttttccgaatgaccttaaaggtgatcggctttaacgaatatgtttacatatgcatagcgctgcactcagtcggactgaacgtagccagaggaaaaaaaaatcatcaaggacaaaggcctcgacctgttgcgctgggccgtctgttccttctaggcactgtatttaactaacttta

SEQ ID NO: 8 - protein, 2725 aa, Homo sapiens
MDVKERRPYCSLTKSRREKERRYTNSSADNEECRVPTQKSYSSSETLKAFDHDSSRLLYGNRVKDLVHREADEFTRQGQNFTLRQLGVCEPATRRGLAFCAEMGLPHRGYSISAGSDADTENEAVMSPEHAMRLWGRGVKSGRSSCLSSRSNSALTLTDTEHENKSDSENEQPASNQGQSTLQPLPPSHKQHSAQHHPSITSLNRNSLTNRRNQSPAPPAALPAELQTTPESVQLQDSWVLGSNVPLESRHFLFKTGTGTTPLFSTATPGYTMASGSVYSPPTRPLPRNTLSRSAFKFKKSSKYCSWKCTALCAVGVSVLLAILLSYFIAMHLFGLNWQLQQTENDTFENGKVNSDTMPTNTVSLPSGDNGKLGGFTQENNTIDSGELDIGRRAIQEIPPGIFWRSQLFIDQPQFLKFNISLQKDALIGVYGRKKLPPSHTQSSPQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACSSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKDKIGYKEGCPGLCNSNGRCTLDQNGGHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMEKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELRNKDFRHSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLSAPSSLAASPDGTLYIADLGNIRIRAVSKNKPLLNSMNFYEVASPTDQELYIFDINGTHQYTVSLVTGDYLYNFSYSNDNDITAVTDSNGNTLRIRRDPNRMPVRVVSPDNQVIWLTIGTNGCLKGMTAQGLELVLFTYHGNSGLLATKSDETGWTTFFDYDSEGRLTNVTFPTGVVTNLHGDMDKAITVDIESSSREEDVSITSNLSSIDSFYTMVQDQLRNSYQIGYDGSLRIIYASGLDSHYQTEPHVLAGTANPTVAKRNMTLPGENGQNLVEWRFRKEQAQGKVNVFGRKLRVNGRNLLSVDFDRTTKTEKIYDDHRKFLLRIAYDTSGHPTLWLPSSKLMAVNVTYSSTGQIASIQRGTTSEKVDYDGQGRIVSRVFADGKTWSYTYLEKSMVLLLHSQRQYIFEYDMWDRLSAITMPSVARHTMQTIRSIGYYRNIYNPPESNASIITDYNEEGLLLQTAFLGTSRRVLFKYRRQTRLSEILYDSTRVSFTYDETAGVLKTVNLQSDGFICTIRYRQIGPLIDRQIFRFSEDGMVNARFDYSYDNSFRVTSMQGVINETPLPIDLYQFDDISGKVEQFGKFGVIYYDINQIISTAVMTYTKHFDAHGRIKEIQYEIFRSLMYWITIQYDNMGRVTKREIKIGPFANTTKYAYEYDVDGQLQTVYLNEKIMWRYNYDLNGNLHLLNPSNSARLTPLRYDLRDRITRLGDVQYRLDEDGFLRQRGTEIFEYSSKGLLTRVYSKGSGWTVIYRYDGLGRRVSSKTSLGQHLQFFYADLTYPTRITHVYNHSSSEITSLYYDLQGHLFAMEISSGDEFYIASDNTGTPLAVFSSNGLMLKQIQYTAYGEIYFDSNIDFQLVIGFHGGLYDPLTKLIHFGERDYDILAGRWTTPDIEIWKRIGKDPAPFNLYMFRNNNPASKIHDVKDYITDVNSWLVTFGFHLHNAIPGFPVPKFDLTEPSYELVKSQQWDDIPPIFGVQQQVARQAKAFLSLGKMAEVQVSRRRAGGAQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGRR

SEQ ID NO: 9 - DNA, 8645 bp, Homo sapiens
  CDS (151)..(8313)
tttggcctcgggccagaattcggcacgaggggtctggagcttggaggagaagtctgaactaaggataaactaaagagaggccaatgagacttgaaccctgagcctaagttgtcaccagcaggactgatgtgcacacagaaggaatgaagtatggatgtgaaagaacgcaggccttactgctccctgaccaagagcagacgagagaaggaacggcgctacacaaattcctccgcagacaatgaggagtgccgggtacccacacagaagtcctacagttccagcgagacattgaaagcttttgatcatgattcctcgcggctgctttacggcaacagagtgaaggatttggttcacagagaagcagacgagttcactagacaaggacagaattttaccctaaggcagttaggagtttgtgaaccagcaactcgaagaggactggcattttgtgcggaaatggggctccctcacagaggttactctatcagtgcagggtcagatgctgatactgaaaatgaagcagtgatgtccccagagcatgccatgagactttggggcaggggggtcaaatcaggccgcagctcctgcctgtcaagtcggtccaactcagccctcaccctgacagatacggagcacgaaaacaagtccgacagtgagaatgagcaacctgcaagcaatcaaggccagtctaccctgcagcccttgccgccttcccataagcagcactctgcacagcatcatccatccatcacttctctcaacagaaactccctgaccaatagaaggaaccagagtccggccccgccggctgctttgcccgccgagctgcaaaccacacccgagtccgtccagctgcaggacagctgggtccttggcagtaatgtaccactggaaagcaggcatttcctattcaaaacaggaacaggtacaacgccactgttcagtactgcaaccccaggatacacaatggcatctggctctgtttattcaccacctactcggccactacctagaaacaccctatcaagaagtgcttttaaattcaagaagtcttcaaagtactgtagctggaaatgcactgcactgtgtgccgtaggggtctcggtgctcctggcaatactcctgtcttattttatagcaatgcatctctttggcctcaactggcagctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaaaggcttaccgccttcccatactcagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcctgtgcttgcagctcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagacaagataggatataaagagggttgtcctggtctgtgcaacagcaatggaagatgtaccctggaccaaaatggcggacattgtgtgtgccagcctggatggagaggagcaggctgtgacgtagccatggagactctttgcacagatagcaaggacaatgaaggggatggactcattgactgcatggatcccgattgctgcctacagagttcctgccagaatcagccctattgtcggggactgccggatcctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatggagaaagaagagaatgacattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcatcatgggcaatgggcgaaggcgcagcatttcctgccccagttgcaatggtcaagctgatggtaacaagttactggccccagtggcgctagcttgtgggatcgatggcagtctgtacgtaggcgatttcaactacgtgcggcggatattcccttctggaaatgtaacaagtgtcttagaactaagaaataaagattttagacatagcagcaacccagctcatagatactaccttgcaacggatccagtcacgggagatctgtacgtttctgacacaaacacccgcagaatttatcgcccaaagtcacttacgggggcaaaagacttgactaaaaatgcagaagtcgtcgcagggacaggggagcaatgccttccgtttgacgaggcgagatgtggggatggagggaaggccgtggaagccacactcatgagtcccaaaggaatggcagttgataagaatggattaatctactttgttgatggaaccatgattaggaaagttgaccaaaatggaatcatatcaactcttctgggctctaacgatttgacttcagccagacctttaacttgtgacaccagcatgcacatcagccaggtacgtctggaatggcccactgacctagccattaaccctatggataactccatttatgtcctggataataatgtagttttacagatcactgaaaatcgtcaagttcgcattgctgctggacggcccatgcactgtcaggttcccggagtggaatatcctgtggggaagcacgcggtgcagacaacactggaatcagccactgccattgctgtgtcctacagtggggtcctgtacattactgaaactgatgagaagaaaattaaccggataaggcaggtcacaacagatggagaaatctccttagtggccggaataccttcagagtgtgactgcaaaaatgatgccaactgtgactgttaccagagtggagatggctacgccaaggatgccaaactcagtgccccatcctccctggctgcttctccagatggtacactgtatattgcagatctagggaatatccggatccgggctgtgtcaaagaataagcctttacttaactctatgaacttctatgaagttgcgtctccaactgatcaagaactctacatctttgacatcaatggtactcaccaatatactgtaagtttagtcactggtgattacctttacaattttagctacagcaatgacaatgatattactgctgtgacagacagcaatggcaacacccttagaattagacgggacccaaatcgcatgccagttcgagtggtgtctcctgataaccaagtgatatggttgacaataggaacaaatggatgtttgaaaggcatgactgctcaaggactggaattagttttgtttacttaccatggcaatagtggccttttagccactaaaagtgatgaaactggatggacaacgttttttgactatgacagtgaaggtcgtctgacaaatgttacgtttccaactggagtggtcacaaacctgcatggggacatggacaaggctatcacagtggacattgagtcatctagccgagaagaagatgtcagcatcacttcaaatctgtcctcgatcgattctttctacaccatggttcaagatcagttaagaaacagctaccagattggttatgacggctccctcagaattatctacgccagtggcctggactcacactaccaaacagagccgcacgttctggctggcaccgctaatccgacggttgccaaaagaaacatgactttgcctggcgagaacggtcaaaacttggtggaatggagattccgaaaagagcaagcccaagggaaagtcaatgtctttggccgcaagctcagggttaatggcagaaacctcctttcagttgactttgatcgaacaacaaagacagaaaagatctatgacgaccaccgtaaatttctactgaggatcgcctacgacacgtctgggcacccgactctctggctgccaagcagcaagctgatggccgtcaatgtcacctattcatccacaggtcaaattgccagcatccagcgaggcaccactagcgagaaagtagattatgacggacaggggaggatcgtgtctcgggtctttgctgatggtaaaacatggagttacacatatttagaaaagtccatggttcttctgcttcatagccagcggcagtacatcttcgaatacgatatgtgggaccgcctgtctgccatcaccatgcccagtgtggctcgccacaccatgcagaccatccgatccattggctactaccgcaacatatacaaccccccggaaagcaacgcctccatcatcacggactacaacgaggaagggctgcttctacaaacagctttcttgggtacaagtcggagggtcttattcaaatacagaaggcagactaggctctcagaaattttatatgatagcacaagagtcagttttacctatgatgaaacagcaggagtcctaaagacagtaaacctccagagtgatggttttatttgcaccattagatacaggcaaattggtcccctgattgacaggcagattttccgctttagtgaagatgggatggtaaatgcaagatttgactatagctatgacaacagctttcgagtgaccagcatgcagggtgtgatcaatgaaacgccactgcctattgatctgtatcagtttgatgacatttctggcaaagttgagcagtttggaaagtttggagttatatattatgatattaaccagatcatttctacagctgtaatgacctatacgaagcactttgatgctcatggccgtatcaaggagattcaatatgagatattcaggtcgctcatgtactggattacaattcagtatgataacatgggtcgggtaaccaagagagagattaaaatagggccctttgccaacaccaccaaatatgcttatgaatatgatgttgatggacagctccaaacagtttacctcaatgaaaagataatgtggcggtacaactacgatctgaatggaaacctccatttactgaacccaagtaacagtgcgcgtctgacaccccttcgctatgacctgcgagacagaatcactcgactgggtgatgttcaatatcggttggatgaagatggtttcctacgtcaaaggggcacggaaatctttgaatatagctccaaggggcttctaactcgagtttacagtaaaggcagtggctggacagtgatctaccgttatgacggcctgggaaggcgtgtttctagcaaaaccagtctaggacagcacctgcagtttttttatgctgacttaacttatcccactaggattactcatgtctacaaccattcgagttcagaaattacctccctgtattatgatctccaaggacatctttttgccatggaaatcagcagtggggatgaattctatattgcatcggataacacagggacaccactggctgtgttcagtagcaatgggcttatgctgaaacagattcagtacactgcatatggggaaatctattttgactctaatattgactttcaactggtaattggatttcatggtggcctgtatgacccactcaccaaattaatccactttggagaaagagattatgacattttggcaggacggtggacaacacctgacatagaaatctggaaaagaattgggaaggacccagctccttttaacttgtacatgtttaggaataacaaccctgcaagcaaaatccatgacgtgaaagattacatcacagatgttaacagctggctggtgacatttggtttccatctgcacaatgctattcctggattccctgttcccaaatttgatttaacagaaccttcttacgaacttgtgaagagtcagcagtgggatgatataccgcccatcttcggagtccagcagcaagtggcgcggcaggccaaggccttcctgtcgctggggaagatggccgaggtgcaggtgagccggcgccgggccggcggcgcgcagtcctggctgtggttcgccacggtcaagtcgctgatcggcaagggcgtcatgctggccgtcagccagggccgcgtgcagaccaacgtgctcaacatcgccaacgaggactgcatcaaggtggcggccgtgctcaacaacgccttctacctggagaacctgcacttcaccatcgagggcaaggacacgcactacttcatcaagaccaccacgcccgagagcgacctgggcacgctgcggttgaccagcggccgcaaggcgctggagaacggcatcaacgtgacggtgtcgcagtccaccacggtggtgaacggcaggacgcgcaggttcgcggacgtggagatgcagttcggcgcgctggcgctgcacgtgcgctacggcatgaccctggacgaggagaaggcgcgcatcctggagcaggcgcggcagcgcgcgctcgcccgggcctgggcgcgcgagcagcagcgcgtgcgcgacggcgaggagggcgcgcgcctctggacggagggcgagaagcggcagctgctgagcgccggcaaggtgcagggctacgacgggtactacgtactctcggtggagcagtaccccgagctggccgacagcgccaacaacatccagttcctgcggcagagcgagatcggcaggaggtaacgcccgggccgcgcccgccgagccgctcacgccctgcccacattgtcctgtggcacaacccgagtgggactctccaacgcccaagagccttcctcccgggggaatgagactgctgttacgacccacacccacaccgcgaaaacaaggaccgcttttttccgaatgaccttaaaggtgatcggctttaacgaatatgtttacatatgcatagcgctgcactcagtcggactgaacgtagccagaggaaaaaaaaatcatcaaggacaaaggcctcgacctgttgcgctgggccgtctgttccttctaggcactgtatttaactaacttta

SEQ ID NO: 10 - protein, 2721 aa, Homo sapiens
MDVKERRPYCSLTKSRREKERRYTNSSADNEECRVPTQKSYSSSETLKAFDHDSSRLLYGNRVKDLVHREADEFTRQGQNFTLRQLGVCEPATRRGLAFCAEMGLPHRGYSISAGSDADTENEAVMSPEHAMRLWGRGVKSGRSSCLSSRSNSALTLTDTEHENKSDSENEQPASNQGQSTLQPLPPSHKQHSAQHHPSITSLNRNSLTNRRNQSPAPPAALPAELQTTPESVQLQDSWVLGSNVPLESRHFLFKTGTGTTPLFSTATPGYTMASGSVYSPPTRPLPRNTLSRSAFKFKKSSKYCSWKCTALCAVGVSVLLAILLSYFIAMHLFGLNWQLQQTENDTFENGKVNSDTMPTNTVSLPSGDNGKLGGFTQENNTIDSGELDIGRRAIQEIPPGIFWRSQLFIDQPQFLKFNISLQKDALIGVYGRKGLPPSHTQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACSSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKDKIGYKEGCPGLCNSNGRCTLDQNGGHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMEKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELRNKDFRHSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDNNVVLQITENRQVRIAAGRPMHCQVPGVEYPVGKHAVQTTLESATAIAVSYSGVLYITETDEKKINRIRQVTTDGEISLVAGIPSECDCKNDANCDCYQSGDGYAKDAKLSAPSSLAASPDGTLYIADLGNIRIRAVSKNKPLLNSMNFYEVASPTDQELYIFDINGTHQYTVSLVTGDYLYNFSYSNDNDITAVTDSNGNTLRIRRDPNRMPVRVVSPDNQVIWLTIGTNGCLKGMTAQGLELVLFTYHGNSGLLATKSDETGWTTFFDYDSEGRLTNVTFPTGVVTNLHGDMDKAITVDIESSSREEDVSITSNLSSIDSFYTMVQDQLRNSYQIGYDGSLRIIYASGLDSHYQTEPHVLAGTANPTVAKRNMTLPGENGQNLVEWRFRKEQAQGKVNVFGRKLRVNGRNLLSVDFDRTTKTEKIYDDHRKFLLRIAYDTSGHPTLWLPSSKLMAVNVTYSSTGQIASIQRGTTSEKVDYDGQGRIVSRVFADGKTWSYTYLEKSMVLLLHSQRQYIFEYDMWDRLSAITMPSVARHTMQTIRSIGYYRNIYNPPESNASIITDYNEEGLLLQTAFLGTSRRVLFKYRRQTRLSEILYDSTRVSFTYDETAGVLKTVNLQSDGFICTIRYRQIGPLIDRQIFRFSEDGMVNARFDYSYDNSFRVTSMQGVINETPLPIDLYQFDDISGKVEQFGKFGVIYYDINQIISTAVMTYTKHFDAHGRIKEIQYEIFRSLMYWITIQYDNMGRVTKREIKIGPFANTTKYAYEYDVDGQLQTVYLNEKIMWRYNYDLNGNLHLLNPSNSARLTPLRYDLRDRITRLGDVQYRLDEDGFLRQRGTEIFEYSSKGLLTRVYSKGSGWTVIYRYDGLGRRVSSKTSLGQHLQFFYADLTYPTRITHVYNHSSSEITSLYYDLQGHLFAMEISSGDEFYIASDNTGTPLAVFSSNGLMLKQIQYTAYGEIYFDSNIDFQLVIGFHGGLYDPLTKLIHFGERDYDILAGRWTTPDIEIWKRIGKDPAPFNLYMFRNNNPASKIHDVKDYITDVNSWLVTFGFHLHNAIPGFPVPKFDLTEPSYELVKSQQWDDIPPIFGVQQQVARQAKAFLSLGKMAEVQVSRRRAGGAQSWLWFATVKSLIGKGVMLAVSQGRVQTNVLNIANEDCIKVAAVLNNAFYLENLHFTIEGKDTHYFIKTTTPESDLGTLRLTSGRKALENGINVTVSQSTTVVNGRTRRFADVEMQFGALALHVRYGMTLDEEKARILEQARQRALARAWAREQQRVRDGEEGARLWTEGEKRQLLSAGKVQGYDGYYVLSVEQYPELADSANNIQFLRQSEIGRR

SEQ ID NO: 11 - DNA, 783 bp, Homo sapiens
  CDS (7)..(777)
aagctttgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcctgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgagggttgtcctggtctgtgcaacagcaatggaagatgtaccctggaccaaaatggctggcattgtgtgtgccagcctggatggagaggagcaggctgtgacgtcgac

SEQ ID NO: 12 - protein, 257 aa, Homo sapiens
CPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIEGCPGLCNSNGRCTLDQNGWHCVCQPGWRGAGCD

SEQ ID NO: 13 - DNA, 1833 bp, Homo sapiens
  CDS (7)..(1827)
aagcttgaccaaaatggcggacattgtgtgtgccagcctggatggagaggagcaggctgtgacgtagccatggagactctttgcacagatagcaaggacaatgaaggagatggactcattgactgcatggatcccgattgctgcctacagagttcctgccagaatcagccctattgtcggggactgccggatcctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatgaagaaagaagagaatgacattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcatcatgggcaatgggcgaaggcgcagcatttcctgccccagttgcaatggtcaagctgatggtaacaagttactggccccagtggcgctagcttgtgggatcgatggcagtctgtacgtaggcgatttcaactatgtgcggcggatattcccttctggaaatgtaacaagtgtcttagaactaagcagcaacccagctcatagatactaccttgcaacggatccagtcacgggagatctgtacgtttctgacacaaacacccgcagaatttatcgcccaaagtcacttacgggggcaaaagacttgactaaaaatgcagaagtcgtcgcagggacaggggagcaatgccttccgtttgacgaggcgagatgtggggatggagggaaggccgtggaagccacactcatgagtcccaaaggaatggcagttgataagaatggattaatctactttgttgatggaaccatgattaggaaagttgaccaaaatggaatcatatcaactcttctgggttctaacgatttgacttcagccagacctttaacttgtgacaccagcatgcacatcagccaggtacgtctggaatggcccactgacctagccattaaccctatggataactccatttatgtcctggataatgtcgac

SEQ ID NO: 14 - protein, 607 aa, Homo sapiens
DQNGGHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMKKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSIMGNGRRRSISCPSCNGQADGNKLLAPVALACGIDGSLYVGDFNYVRRIFPSGNVTSVLELSSNPAHRYYLATDPVTGDLYVSDTNTRRIYRPKSLTGAKDLTKNAEVVAGTGEQCLPFDEARCGDGGKAVEATLMSPKGMAVDKNGLIYFVDGTMIRKVDQNGIISTLLGSNDLTSARPLTCDTSMHISQVRLEWPTDLAINPMDNSIYVLDN

SEQ ID NO: 15 - DNA, 768 bp, Homo sapiens
  CDS (65)..(706)
agacttgaaccctgagcctaagttgtcaccagcaggactgatgtgcacacagaaggaatgaagtatggatgtgaaagaacgcaggccttactgctccctgaccaagagcagacgagagaaggaacggcgctacacaaattcctccgcagacaatgaggagtgccgggtacccacacagaagtcctacagttccagcgagacattgaaagcttttgatcatgattcctcgcggctgctttacggcaacagagtgaaggatttggttcacagagaagcagacgagttcactagacaaggacagaattttaccctaaggcagttaggagtttgtgaaccagcaactcgaagaggactggcattttgtgcggaaatggggctccctcacagaggttactctatcagtgcagggtcagatgctgatactgaaaatgaagcagtgatgtccccagagcatgccatgagactttggggcaggggggtcaaatcaggccgcagctcctgcctgtcaagtcggtccaactcagccctcaccctgacagatacggagcacgaaaacaagtccgacagtgagaatggagggtcaagcagttggttcggttttcattggaatttttatgtgggtaaagcttcctgtttgctgcgcttgcctaggattttcttatcccacaactacaatgtgaacaaagagatgagagagaaattatgctaatgcattttggtggatcaatgctaatgcattttggtggatcaatgctaatgcattttggt

SEQ ID NO: 16 - protein, 214 aa, Homo sapiens
MDVKERRPYCSLTKSRREKERRYTNSSADNEECRVPTQKSYSSSETLKAFDHDSSRLLYGNRVKDLVHREADEFTRQGQNFTLRQLGVCEPATRRGLAFCAEMGLPHRGYSISAGSDADTENEAVMSPEHAMRLWGRGVKSGRSSCLSSRSNSALTLTDTEHENKSDSENGGSSSWFGFHWNFYVGKASCLLRLPRIFLSHNYNVNKEMREKLC

SEQ ID NO: 17 - DNA, 891 bp, Homo sapiens
  CDS (1)..(888)
gacgcggcccagccggccaggcgcgcgcgccgtacgaagctttgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcctgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgagggttgtcctggtctgtgcaacagcaatggaagatgtaccctggaccaaaatggctggcattgtgtgtgccagcctggatggagaggagcaggctgtgacctcgagggtaagcctatccctaaccctctcctcggtctcgattctacgcgtaccggtcatcatcaccatcaccattga

SEQ ID NO: 18 - protein, 296 aa, Homo sapiens
DAAQPARRARRTKLCPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIEGCPGLCNSNGRCTLDQNGWHCVCQPGWRGAGCDLEGKPIPNPLLGLDSTRTGHHHHHH

SEQ ID NO: 19 - DNA, 2589 bp, Homo sapiens
  CDS (1)..(2586)
gacgcggcccagccggccaggcgcgcgcgccgtacgaagctttcgcgaaactggcagctacagcagactgaaaatgacacatttgagaatggaaaagtgaattctgataccatgccaacaaacactgtgtcattaccttctggagacaatggaaaattaggtggatttacgcaagaaaataacaccatagattccggagaacttgatattggccgaagagcaattcaagagattcctcccgggatcttctggagatcacagctcttcattgatcagccacagtttcttaaattcaatatctctcttcagaaggatgcattgattggagtatatggccggaaaggcttaccgccttcccatactcagtatgacttcgtggagctcctggatggcagcaggctgattgccagagagcagcggagcctgcttgagacggagagagccgggcggcaggcgagatccgtcagccttcatgaggccggctttatccagtacttggattctggaatctggcatctggctttttataatgatgggaaaaatgcagagcaggtgtcttttaataccattgttatagagtctgtggtggaatgtccccgaaattgccatggaaatggagaatgcgtttctggaacttgccattgttttccaggatttctgggtccggattgttcaagagccgcctgtccagtgttatgtagtggcaacgggcagtactccaagggccgctgcctgtgtttcagcggctggaagggcaccgagtgtgatgtgccgactacccagtgtattgacccacagtgtgggggtcgtgggatttgtatcatgggctcttgtgcttgcaactcaggatacaaaggagaaagttgtgaagaagctgactgtatagaccctgggtgttctaatcatggtgtgtgtatccacggggaatgtcactgcagtccaggatggggaggtagcaattgtgaaatactgaagaccatgtgtccagaccagtgctccggccacggaacgtatcttcaagaaagtggctcctgcacgtgtgaccctaactggactggcccagactgctcaaacgaaatatgttctgtggactgtggctcacacggcgtttgcatgggggggacgtgtcgctgtgaagaaggctggacgggcccagcctgtaatcagagagcctgccacccccgctgtgccgagcacgggacctgcaaggatggcaagtgtgaatgcagccagggctggaatggagagcactgcactatcgctcactatttggataagatagttaaagagggttgtcctggtctgtgcaacagcaatggaagatgtaccctggaccaaaatggctggcattgtgtgtgccagcctggatggagaggagcaggctgtgacgtagccatggagactctttgcacagatagtaaggacaatgaaggagatggactcattgactgcatggatcccgattgctgcctacagagttcctgccagaatcagccctattgtcggggactgccggatcctcaggacatcattagccaaagccttcaatcgccttctcagcaagctgccaaatccttttatgatcgaatcagtttccttataggatctgatagcacccatgttatacctggagaaagtcctttcaataagagccttgcatctgtcatcagaggccaagtactgactgctgatggaactccacttattggagtaaatgtctcgtttttccattacccagaatatggatatactattacccgccaggacggaatgtttgacttggtggcaaatggtggggcctctctaactttggtatttgaacgatccccattcctcactcagtatcatactgtgtggattccatggaatgtcttttatgtgatggataccctagtcatgaagaaagaagagaatgacattcccagctgtgatctgagtggattcgtgaggccaaatcccatcattgtgtcatcacctttatccacctttttcagatcttctcctgaagacagtcccatcattcccgaaacacaggtactccacgaggaaactacaattccaggaacagatttgaaactctcctacttgagttccagagctgcagggtataagtcagttctcaagatcaccatgacccagtctattattccatttaatttaatgaaggttcatcttatggtagctgtagtaggaagactcttccaaaagtggtttcctgcctcaccaaacttggcctatactttcatatgggataaaacagatgcatataatcagaaagtctatggtctatctgaagctgttgtgtcagttggatatgagtatgagtcgtgtttggacctgactctgtgggaaaagaggactgccattctgcagggctatgaattggatgcgtccaacatgggtggctggacattagataaacatcacgtgctggatgtacagaacggtatactgtacaagggaaacggggaaaaccagttcatctcccagcagcctccagtcgtgagtagcctcgagggtaagcctatccctaaccctctcctcggtctcgattctacgcgtaccggtcatcatcaccatcaccattga

SEQ ID NO: 20 - protein, 862 aa, Homo sapiens
DAAQPARRARRTKLSRNWQLQQTENDTFENGKVNSDTMPTNTVSLPSGDNGKLGGFTQENNTIDSGELDIGRRAIQEIPPGIFWRSQLFIDQPQFLKFNISLQKDALIGVYGRKGLPPSHTQYDFVELLDGSRLIAREQRSLLETERAGRQARSVSLHEAGFIQYLDSGIWHLAFYNDGKNAEQVSFNTIVIESVVECPRNCHGNGECVSGTCHCFPGFLGPDCSRAACPVLCSGNGQYSKGRCLCFSGWKGTECDVPTTQCIDPQCGGRGICIMGSCACNSGYKGESCEEADCIDPGCSNHGVCIHGECHCSPGWGGSNCEILKTMCPDQCSGHGTYLQESGSCTCDPNWTGPDCSNEICSVDCGSHGVCMGGTCRCEEGWTGPACNQRACHPRCAEHGTCKDGKCECSQGWNGEHCTIAHYLDKIVKEGCPGLCNSNGRCTLDQNGWHCVCQPGWRGAGCDVAMETLCTDSKDNEGDGLIDCMDPDCCLQSSCQNQPYCRGLPDPQDIISQSLQSPSQQAAKSFYDRISFLIGSDSTHVIPGESPFNKSLASVIRGQVLTADGTPLIGVNVSFFHYPEYGYTITRQDGMFDLVANGGASLTLVFERSPFLTQYHTVWIPWNVFYVMDTLVMKKEENDIPSCDLSGFVRPNPIIVSSPLSTFFRSSPEDSPIIPETQVLHEETTIPGTDLKLSYLSSRAAGYKSVLKITMTQSIIPFNLMKVHLMVAVVGRLFQKWFPASPNLAYTFIWDKTDAYNQKVYGLSEAVVSVGYEYESCLDLTLWEKRTAILQGYELDASNMGGWTLDKHHVLDVQNGILYKGNGENQFISQQPPVVSSLEGKPIPNPLLGLDSTRTGHHHHHH

SEQ ID NO: 21 - DNA, 22 bp, Artificial Sequence
  Description of Artifical Sequence Primer/Probe
cacggaacgtatcttcaagaaa

SEQ ID NO: 22 - DNA, 26 bp, Artificial Sequence
  Description of Artifical Sequence Primer/Probe
ctgcacgtgtgaccctaactggactg

SEQ ID NO: 23 - DNA, 22 bp, Artificial Sequence
  Description of Artifical Sequence Primer/Probe
gccacagtccacagaacatatt

SEQ ID NO: 24 - DNA, 22 bp, Artificial Sequence
  Description of Artifical Sequence Primer/Probe
acctactcggccactacctaga

SEQ ID NO: 25 - DNA, 29 bp, Artificial Sequence
  Description of Artifical Sequence Primer/Probe
caccctatcaagaagtgcttttaaattca

SEQ ID NO: 26 - DNA, 22 bp, Artificial Sequence
  Description of Artifical Sequence Primer/Probe
cagtgcatttccagctacagta

SEQ ID NO: 27 - DNA, 22 bp, Artificial Sequence
  Description of Artifical Sequence Primer/Probe
cagagaagcagacgagttcact

SEQ ID NO: 28 - DNA, 26 bp, Artificial Sequence
  Description of Artifical Sequence Primer/Probe
caaggacagaattttaccctaaggca

SEQ ID NO: 29 - DNA, 22 bp, Artificial Sequence
  Description of Artifical Sequence Primer/Probe
gttgctggttcacaaactccta

SEQ ID NO: 30 - DNA, 20 bp, Artificial Sequence
  Description of Artifical Sequence Primer/Probe
cacctactcggccactacct

SEQ ID NO: 31 - DNA, 29 bp, Artificial Sequence
  Description of Artifical Sequence Primer/Probe
caccctatcaagaagtgcttttaaattca

SEQ ID NO: 32 - DNA, 20 bp, Artificial Sequence
  Description of Artifical Sequence Primer/Probe
cacacagtgcagtgcatttc

SEQ ID NO: 33 - DNA, 22 bp, Artificial Sequence
  Description of Artifical Sequence Primer/Probe
gcagctacagcagactgaaaat

SEQ ID NO: 34 - DNA, 27 bp, Artificial Sequence
  Description of Artifical Sequence Primer/Probe
tctgataccatgccaacaaacactgtg

SEQ ID NO: 35 - DNA, 22 bp, Artificial Sequence
  Description of Artifical Sequence Primer/Probe
ccattgtctccagaaggtaatg

SEQ ID NO: 36 - protein, 14 aa, Homo sapiens
DAAQPARRARRTKL

SEQ ID NO: 37 - protein, 13 aa, Homo sapiens
AAQPARRARRTKL

SEQ ID NO: 38 - protein, 25 aa, Homo sapiens
LEGKPIPNPLLGLDSTRTGHHHHHH

(19) United States
(12) Patent Application Publication
(10) Pub. No.: US 2005/0244868 A1
(43) Pub. Date: Nov. 3, 2005

(54) Ten-M3 polypeptides and polynucleotides and their methods of use

(76) Inventors: Ramesh Kekuda, Durham, NC (US); Timothy MacLachlan, Unionville, CT (US); Meera Patturajan, Caldwell, NJ (US); Luca Rastelli, Guilford, CT (US); Seth Ettenberg, New Haven, CT (US); Corine Vernet, Chernex (CH)

Correspondence Address: CURAGEN CORPORATION, 322 EAST MAIN STREET, BRANFORD, CT, 06405, US

(21) Appl. No.: 11/096,051

(22) Filed: Mar. 30, 2005

Related U.S. Application Data
(63) Continuation-in-part of application No. 10/038,854, filed on Dec. 31, 2001.
(63) Continuation-in-part of application No. 10/455,772, filed on Jun. 4, 2003.
(60) Provisional application No. 60/557,978, filed on Mar. 30, 2004.

(51) Int. Cl. C12Q 1/68; G01N 33/574; C07H 21/04; C07K 14/82; C07K 16/30

(52) U.S. Cl. 435/6; 435/7.23; 435/69.1; 435/320.1; 435/325; 530/350; 530/388.8; 536/23.2

(57) ABSTRACT

Disclosed herein are novel Ten-M3 polynucleotides encoding novel polypeptides and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. patent application Ser. No. 10/038,854 filed Dec. 31, 2001 and Ser. No. 10/455,772 filed Jun. 4, 2003. This application also claims the benefit of U.S. Provisional Application Ser. No. 60/557,978 filed Mar. 30, 2004. The content of each is incorporated herein by reference in its entirety.

### FIELD OF THE INVENTION

The present invention relates to compositions and methods for preventing and treating cancer. More particularly, the present invention relates to compositions comprising Ten-M3, CG55069, a fragment, a derivative, a variant, a homolog, or an analog thereof, and antibodies thereto and their uses in preventing and treating cancer.

### BACKGROUND

The human Ten-M family of genes, also known as teneurins or hOdz, are a class of type II membrane proteins containing a short intracellular N-terminus, a transmembrane region followed extracellularly by 8 epidermal growth factor (EGF)-like repeats and a large globular domain. The EGF repeats found in Ten-M proteins are thought to mediate dimerization which may regulate their function. Drosophila Ten-m protein was originally discovered as the first pair-rule gene that was not a transcription factor. The expression patterns of mouse and chicken homologues of Ten-M proteins suggest a role in neuronal development and neurite outgrowth. The murine family of Ten-m protein homologs consists of at least four members (Ten-m1-4) each possessing similar structural features. Ten-M proteins may bind extracellular matrix proteins such as heparin, indicating a role as a cell adhesion molecule. mRNA levels of human Ten-M proteins, appear to be upregulated in certain cancers, and may be implicated in metastatic cell migration. [Dev. Biol. 216, 195-209 (1999), J. Cell Biol. 145, 563-577 (1999)].

### SUMMARY OF THE INVENTION

The present invention provides compositions comprising CG55069 polypeptide or polynucleotide and antibodies to CG55069 polypeptides. The invention provides methods of preventing angiogenesis and/or cell migration and therefore preventing and/or treating cancer comprising administering to a subject in need thereof a composition comprising one or more CG55069 proteins or an antibody thereto.

In one embodiment, the present invention provides an isolated protein selected from the group consisting of: (a) a protein comprising an amino acid sequence of SEQ ID NOs:2, 4, 6, 8, 10, 12, 14, 16, 18, or 20; (b) a protein with one or more amino acid substitutions to the protein of (a), wherein said substitutions are no more than 15% of the amino acid sequence of SEQ ID NOs: 2, 4, 6, 8, 10, 12, 14, 16, 18, or 20, and wherein said protein with one or more amino acid substitutions retains antiangiogenic activity; and (c) a fragment of the protein of (a) or (b), which fragment retains antiangiogenic and/or inhibits cell migration activity.

In another embodiment, the present invention provides an isolated nucleic acid molecule selected from the group consisting of: (a) a nucleic acid molecule comprising a nucleotide sequence selected from the group consisting of SEQ ID NOs: 1, 3, 5, 7, 9, 11, 13, 15, 17 and 19; (b) a nucleic acid molecule encoding a protein comprising an amino acid sequence selected from the group consisting of SEQ ID NOs: 1, 3, 5, 7, 9, 11, 13, 15, 17 and 19 and (c) a nucleic acid molecule hybridizes under stringent conditions to a nucleotide sequence of SEQ ID NO: 1, 3, 5, 7, 9, 11, 13, 15, 17 or 19 or a complement of said nucleic acid molecule. In a specific embodiment, the stringent conditions comprise a salt concentration from about 0.1 M to about 1.0 M sodium ion, a pH from about 7.0 to about 8.3, a temperature is at least about 60oC., and at least one wash in 0.2×SSC, 0.01% BSA.

In one embodiment, the present invention provides an isolated antibody with specificity to a protein selected from the group consisting of a protein comprising an amino acid sequence of SEQ ID NOs: 2, 4, 6, 8, 10, 12, 14, 16, 18, or 20; or a fragment of the antibody which fragment retains the specific binding activity.

In some specific embodiments, one or more CG55069 proteins are isolated from a cultured eukaryotic cell. In some other specific embodiments, one or more CG55069 proteins are isolated from a cultured prokaryotic cell. In a preferred embodiment, one or more CG55069 proteins are isolated from E. coli. In a specific embodiment, one or more CG55069 proteins isolated from a cultured host cell has a purity of at least 90%, at least 95%, at least 96%, at least 97%, at least 98%, or at least 99%.

In one embodiment, the present invention provides methods of preventing angiogenesis and/or cell migration and therefore preventing and/or treating cancer comprising administering to a subject in need thereof a prophylactically and/or therapeutically effective amount of an isolated protein selected from the group consisting of: (a) a protein comprising an amino acid sequence of SEQ ID NOs: 2, 4, 6, 8, 10, 12, 14, 16, 18, or 20; (b) a protein with one or more amino acid substitutions to the protein of (a), wherein said substitutions are no more than 15% of the amino acid sequence of SEQ ID NOs: 2, 4, 6, 8, 10, 12, 14, 16, 18, or 20, and wherein said protein with one or more amino acid substitutions retains antiangiogenic activity; and (c) a fragment of the protein of (a) or (b), which fragment retains antiangiogenic and/or inhibits cell migration activity.

In another embodiment, the present invention provides methods of preventing angiogenesis and/or cell migration and therefore preventing and/or treating cancer comprising administering to a subject in need thereof a prophylactically or therapeutically effective amount of a protein isolated from a cultured host cell containing an isolated nucleic acid molecule selected from the group consisting of: (a) a nucleic acid molecule comprising a nucleotide sequence selected from the group consisting of SEQ ID NOs: 1, 3, 5, 7, 9, 11, 13, 15, 17 and 19; (b) a nucleic acid molecule encoding a protein comprising an amino acid sequence selected from the group consisting of SEQ ID NOs: 1, 3, 5, 7, 9, 11, 13, 15, 17 and 19; and (c) a nucleic acid molecule hybridizes under stringent conditions to a nucleotide sequence of SEQ ID NO: 1, 3, 5, 7, 9, 11, 13, 15, 17 or 19, or a complement of said nucleic acid molecule.

In accordance to the present invention, preventing angiogenesis and/or cell migration and therefore preventing and/or treating cancers include, but is not limited to, neuroblastoma, renal carcinoma, fibrosarcoma, rhabdosarcoma, glioblastoma, lung cancer or pancreatic cancer. Cell migration, angiogenesis or actin filament formation is inhibited by contacting or introducing to a cell or tissue a composition containing a CG55069 polypeptide, polynucleotide or antibody. The invention also features methods of preventing or alleviating a symptom of cell migration/angiogenesis related disorder in a subject by administering to the subject a CG55069 polypeptide, polypeptide or antibody. Migrating cells or cells influencing angiogenic activity may be normal or cancerous. The cell is an endothelial cell, an epithelial cell, a neuronal cell, a mesenchymal cell or a fibroblast. For example, the cell may be a neuroblastoma cell, a renal carcinoma cell, a fibrosarcoma cell, a rhabdosarcoma cell, a glioblastoma, a lung cancer cell or a pancreatic cancer cell. The subject may be a mammal such as human. The subject is suffering from or at risk of developing cell migration/angiogenesis related disorder. Cell migration/angiogenesis related disorders include for example, diseases that cause neovascularization, cancer such neuroblastoma, renal carcinoma, fibrosarcoma, rhabdosarcoma and pancreatic cancer, wound healing, or tissue regeneration.

The invention further provides chimeric proteins. The chimeric proteins include a first and a second polypeptide. The first polypeptide includes a CG55069 polypeptide. The second polypeptide, for example, is a portion of an immunoglobulin molecule. The portion of the immunoglobulin molecule includes for example the V5 region of the immunoglobulin molecule. For example, a chimeric protein of the invention may be CG55069-18 or CG55069-19.

The invention also provides methods for treating or preventing cancer such as renal cell carcinoma, prostate carcinoma, thyroid carcinoma, ovarian carcinoma, glioblastoma and lung carcinoma, in mammals, by administering a compound that inhibits Ten-M3. Compounds that inhibit Ten-M3 include proteins that bind to Ten-M3 protein. Examples of compounds that bind Ten-M3 include fragments of the protein or Ten-M3 specific antibodies that antagonize the function of endogenous Ten-M3. Specifically, this invention discloses the use of a fragment of the Ten-M3 protein, CG55069-04 and CG55069-11, that inhibits the cell motility function of Ten-M3.

### DESCRIPTION OF THE DRAWINGS

FIG. 1 is a schematic outline of the regions of the Ten-M3 protein and the region expressed and purified and used in the assays described herein.

FIG. 2 shows Coomassie blue stained polyacrylamide gel of CG55069 protein purified from transfected human embryonic kidney cells.

FIG. 3 is a histogram illustrating CG55069 inhibition of A) HUVEC, B) HMVEC, C) 786-0 and D) H1299 cell migration in a dose dependent manner.

FIG. 4 is a histogram illustrating CG55069-11 antiangiogenic activity in terms of effect on A) vessel nodes B) vessel ends and C) vessel length in a matrigel plug assay.

FIG. 5 FACs analysis showing CG55069 binding to 786-0 cells (A) ; in competition with heparin sulfate (B); U87 cells (C) ; and HUVEC cells (D).

FIG. 6 shows results of targeted cell killing in CG55069 expressing (A) and CG55069 negative (B) cells.

### DETAILED DESCRIPTION OF THE INVENTION

The present invention describes the CG55069 polypeptide and antibodies thereto, variants, biologically active fragments and/or derivatives thereof. As used herein, the term “CG55069”, refers to a class of proteins (including peptides and polypeptides) or nucleic acids encoding such proteins or their complementary strands, where the proteins comprise an amino acid sequence of SEQ ID NO: 2, or its fragments, derivatives, variants, homologs, or analogs. In a preferred embodiment, a CG55069 protein retains at least some biological activity of Ten-M3. As used herein, the term “biological activity” means that a CG55069 protein possesses some but not necessarily all the same properties of (and not necessarily to the same degree as) Ten-M3.

A member (e.g., a protein and/or a nucleic acid encoding the protein) of the CG55069 family may further be given an identification name. For example, CG55069-01 (SEQ ID NOs:7 and 8) represents the first identified Ten-M4 (see U.S. patent application Ser. No. 10/038,854); CG55069-17 (SEQ ID NO. 2) represents the full length cloned protein encoded by the nucleic acid molecule SEQ ID NO. 1. A mature polypeptide results by one or more naturally occurring processing steps that may take place within the cell in which the gene product arises. The mature form may arise as a result of cleavage of the N-terminal methionine residue or N-terminal signal sequence, or post-translational modification such as glycosylation, myristylation or phosphorylation. The extracellular domain (ECD) exemplified by CG55069-16 (SEQ ID NO. 4) encompasses the EGF repeats and the C-terminal globular domain. The EGF domain is exemplified by amino acid sequences of CG55069-04 (SEQ ID NO. 12); N-terminal EGF domain is exemplified by amino acid sequences of CG55069-11 (SEQ ID NO. 6). It is shown herein that the EGF domains CG55069-04 and CG55069-11 inhibit endothelial cell migration and reduce angiogenesis and thus could be used in the prevention and/or treatment of cancer.

Table 1 shows a summary of some of the CG55069 family members. In one embodiment, the invention includes a variant of Ten-M3 protein, in which some amino acids residues, e.g., no more than 1%, 2%, 3%, 5%, 10% or 15% of the amino acid sequence of Ten-M3 (SEQ ID NO:2), are changed. In another embodiment, the invention includes nucleic acid molecules that can hybridize to Ten-M3 under stringent hybridization conditions.

TABLE 1
Summary of CG55069 family members

| | SEQ ID NO | | |
|---|---|---|---|
| Internal | (nucleic | SEQ ID NO | |
| Identification | acid) | (amino acid) | Description |
| CG55069-17 | 1 | 2 | Full length Ten-M3 clone |
| CG55069-16 | 3 | 4 | ECD |
| CG55069-11 | 5 | 6 | N-terminal EGF domain |
| CG55069-01 | 7 | 8 | Ten-M3 |
| CG55069-02 | 9 | 10 | Ten-M3 isoform 2 |
| CG55069-04 | 11 | 12 | EGF |
| CG55069-07 | 13 | 14 | Internal |
| CG55069-15 | 15 | 16 | Alternative Ten-M3 clone |
| CG55069-18 | 17 | 18 | EGF with tags |
| CG55069-19 | 19 | 20 | N-terminal EGF with tags |

As used herein, the term “effective amount” refers to the amount of a therapy (e.g., a composition comprising a CG55069 protein) which is sufficient to reduce and/or ameliorate the severity and/or duration of cancer or one or more symptoms thereof, prevent the advancement of, cause regression of, prevent the recurrence, development, or onset of one or more symptoms associated with cancer, or enhance or improve the prophylactic or therapeutic effect(s) of another therapy.

As used herein, the term “Ten-M3” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules that are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the terms “prevent,” “preventing,” and “prevention” refer to the prevention of the recurrence, onset, or development of cancer or one or more symptoms thereof in a subject resulting from the administration of a therapy (e.g., a composition comprising a CG55069 or an antibody thereto), or the administration of a combination of therapies.

As used herein, the term “prophylactically effective amount” refers to the amount of a therapy (e.g., a composition comprising a CG55069 protein) which is sufficient to result in the prevention of the development, recurrence, or onset of cancer or one or more symptoms thereof, or to enhance or improve the prophylactic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has or is at risk of developing cancer. In another embodiment, the subject is a farm animal (e.g., a horse, pig, or cow) or a pet (e.g., a dog or cat) that has or is at risk of developing cancer. The term “subject” is used interchangeably with “patient” in the present invention.

As used herein, the terms “treat,” “treatment,” and “treating” refer to the reduction of the progression, severity, and/or duration of cancer or amelioration of one or more symptoms thereof, wherein such reduction and/or amelioration result from the administration of one or more therapies (e.g., a composition comprising a CG55069 protein or antibody thereto).

As used herein, the term “therapeutically effective amount” refers to the amount of a therapy (e.g., a composition comprising a CG55069 protein), which is sufficient to reduce the severity of, reduce the duration of, prevent the advancement of, cause regression of, ameliorate one or more symptoms associated with, cancer, or enhance or improve the therapeutic effect(s) of another therapy.

The term “antibody,” as used in this disclosure, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind CG55069 specifically. Typically, such fragments would comprise an antigen-binding domain.

The present invention provides for compositions comprising CG55069 for prevention of angiogenesis and/or cell migration and thereby for treatment of cancer. As used herein, the term “CG55069” refers to a class of proteins (including peptides and polypeptides) or nucleic acids encoding such proteins or their complementary strands, where the proteins comprise an amino acid sequence of SEQ ID NO:2, or its fragments, derivatives, variants, homologs, or analogs.

In one embodiment, a CG55069 protein is a variant of Ten-M3. It will be appreciated by those skilled in the art that DNA sequence polymorphisms that lead to changes in the amino acid sequences of the Ten-M3 protein may exist within a population (e.g., the human population). Such genetic polymorphism in the Ten-M3 gene may exist among individuals within a population due to natural allelic variation. Such natural allelic variations can typically result in 1-5% variance in the nucleotide sequence of the Ten-M3 gene. Any and all such nucleotide variations and resulting amino acid polymorphisms in the Ten-M3 protein, which are the result of natural allelic variation of the Ten-M3 protein, are intended to be within the scope of the invention In another embodiment, the invention provides a fragment of a Ten-M3 protein, including fragments of variant Ten-M3 proteins, mature Ten-M3 proteins, and variants of mature Ten-M3 proteins, as well as Ten-M3 proteins encoded by allelic variants and single nucleotide polymorphisms of Ten-M3 nucleic acids. An example of an Ten-M3 protein fragment includes, but is not limited to, residues 1-308, 1-544, 1-575, 1-609, 1-641, 1-676, 1-685, 1-707, 1-730, 1-738, 1-782, 1-1209, 1-1324, 1-1384, 1-1445, 1-1514, 1-1600, 1-1664, 1-1801, 1-1875, 1-1982, 1-2207, 1-2261, 1-2715, 10-308, 10-544, 10-575, 10-609, 10-641, 10-676, 10-685, 10-707, 10-730, 10-738, 1-782, 10-1209, 10-1324, 10-1384, 10-1445, 10-1514, 10-1600, 10-1664, 10-1801, 10-1875, 10-1982, 10-2207, 10-2261, 10-2715, 325-544, 325-575, 325-609, 325-641, 325-676, 325-685, 325-707, 325-730, 325-738, 325-782, 325-1209, 325-1324, 325-1384, 325-1445, 325-1514, 325-1600, 325-1664, 325-1801, 325-1875, 325-1982, 325-2207, 325-2261, 325-2715, 518-544, 518-575, 518-609, 518-641, 518-676, 518-685, 518-707, 518-730, 518-738, 518-782, 518-1209, 518-1324, 518-1384, 518-1445, 518-1514, 518-1600, 518-1664, 518-1801, 518-1875, 518-1982, 518-2207, 518-2261, 518-2715, 783-1209, 783-1324, 783-1384, 783-1445, 783-1514, 783-1600, 783-1664, 783-1801, 783-1875, 783-1982, 783-2207, 783-2261, 783-2715, of SEQ ID NO:2 or the equivalent of SEQ ID NOs:4, 6, 8, 10, 12, 14, 16, 18, 20. Domain boundaries are somewhat imprecise and can vary by up to ±5 residues from the specified positions.

The invention also encompasses derivatives and analogs of Ten-M3. The production and use of derivatives and analogs related to Ten-M3 are within the scope of the present invention.

In a specific embodiment, the derivative or analog is functionally active, i.e., capable of exhibiting one or more functional activities associated with a full-length, wild-type Ten-M3. Derivatives or analogs of Ten-M3 can be tested for the desired activity by procedures known in the art, including but not limited to, using appropriate cell lines, animal models, and clinical trials.

In particular, Ten-M3 derivatives can be made via altering Ten-M3 sequences by substitutions, insertions or deletions that provide for functionally equivalent molecules. In one embodiment, such alteration of an Ten-M3 sequence is done in a region that is not conserved in the Ten-M3 protein family. Due to the degeneracy of nucleotide coding sequences, other DNA sequences which encode substantially the same amino acid sequence as Ten-M3 may be used in the practice of the present invention. These include, but are not limited to, nucleic acid sequences comprising all or portions of Ten-M3 which are altered by the substitution of different codons that encode a functionally equivalent amino acid residue within the sequence, thus producing a silent change. In a preferred embodiment, a wild-type Ten-M3 nucleic acid sequence is SEQ ID NO:1. Likewise, the Ten-M3 derivatives of the invention include, but are not limited to, those containing, as a primary amino acid sequence, all or part of the amino acid sequence of Ten-M3 including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Ten-M3 derivatives of the invention also include, but are not limited to, those containing, as a primary amino acid sequence, all or part of the amino acid sequence of Ten-M3 including altered sequences in which amino acid residues are substituted for residues with similar chemical properties. In a specific embodiment, 1, 2, 3, 4, or 5 amino acids are substituted.

Derivatives or analogs of Ten-M3 include, but are not limited to, those proteins which are substantially homologous to Ten-M3 or fragments thereof, or whose encoding nucleic acid is capable of hybridizing to the Ten-M3 nucleic acid sequence.

The Ten-M3 derivatives and analogs of the invention can be produced by various methods known in the art. The manipulations which result in their production can occur at the gene or protein level. For example, the cloned Ten-M3 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of Ten-M3, care should be taken to ensure that the modified gene remains within the same translational reading frame as Ten-M3, uninterrupted by translational stop signals, in the gene region where the desired Ten-M3 activity is encoded.

Additionally, the Ten-M3-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the Ten-M3 sequence may also be made at the protein level. Included within the scope of the invention are Ten-M3 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of Ten-M3 can be chemically synthesized. For example, a protein corresponding to a portion of Ten-M3 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the Ten-M3 sequence. Non-classical amino acids include, but are not limited to, the D-isomers of the common amino acids, a-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the Ten-M3 derivative is a chimeric or fusion protein comprising Ten-M3 or a fragment thereof fused via a peptide bond at its amino- and/or carboxy-terminus to a non-Ten-M3 amino acid sequence. In one embodiment, the non-Ten-M3 amino acid sequence is fused at the amino-terminus of an Ten-M3 or a fragment thereof. In another embodiment, such a chimeric protein is produced by recombinant expression of a nucleic acid encoding the protein (comprising an Ten-M3-coding sequence joined in-frame to a non-Ten-M3 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding Ten-M3 with a heterologous signal sequence is expressed such that the chimeric protein is expressed and processed by the cell to the mature Ten-M3 protein. The primary sequence of Ten-M3 and non-Ten-M3 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of Ten-M3 molecule fused to a heterologous (non-Ten-M3) protein-encoding sequence may be constructed. In a specific embodiment, such chimeric construction can be used to enhance one or more desired properties of an Ten-M3, including but not limited to, Ten-M3 stability, solubility, or resistance to proteases. In another embodiment, chimeric construction can be used to target Ten-M3 to a specific site. In yet another embodiment, chimeric construction can be used to identify or purify an Ten-M3 of the invention, such as a His-tag, a FLAG tag, a green fluorescence protein (GFP), β-galactosidase, a maltose binding protein (MalE), a cellulose binding protein (CenA) or a mannose protein, etc.

In some embodiments, a CG55069 protein can be modified so that it has improved solubility and/or an extended half-life in vivo using any methods known in the art. For example, Fc fragment of human IgG, or inert polymer molecules such as high molecular weight polyethyleneglycol (PEG) can be attached to a CG55069 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG55069 protein. Unreacted PEG can be separated from CG55069-PEG conjugates by size-exclusion or by ion-exchange chromatography. PEG-derivatized conjugates can be tested for in vivo efficacy using methods known to those of skill in the art.

A CG55069 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG55069 refers to: CG55069-17 (SEQ ID NOs:1 and 2), G55069-16 (SEQ ID NOs:3 and 4), CG55069-11 (SEQ ID NOs:5 and 6), CG55069-01 (SEQ ID NOs:7 and 8), CG55069-02 (SEQ ID NOs:9 and 10), CG55069-04 (SEQ ID NOs:11 and 12), CG55069-07 (SEQ ID NOs:13 and 14), CG55069-15 (SEQ ID NOs:15 and 16), CG55069-18 (SEQ ID NOs:17 and 18), and CG55069-19 (SEQ ID NOs:19 and 20) or a combination thereof.

Methods of Preparing CG55069

Methods of isolating a CG55069 protein are described in previous applications, e.g., U.S. patent application Ser. No. 10/038,854, the content of which is incorporated herein by reference. Any techniques known in the art can be used in purifying a CG55069 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG55069 may be monitored by one or more in vitro or in vivo assays. The purity of CG55069 can be assayed by any methods known in the art, such as but not limited to, gel electrophoresis. See Scopes, supra. In some embodiment, the CG55069 proteins employed in a composition of the invention can be in the range of 80 to 100 percent of purity, or at least 80%, at least 85%, at least 90%, at least 95%, or at least 98% of purity. In one embodiment, one or more CG55069 proteins employed in a composition of the invention has a purity of at least 99%. In another embodiment, CG55069 is purified to apparent homogeneity, as assayed, e.g., by sodium dodecyl sulfate polyacrylamide gel electrophoresis.

Methods known in the art can be utilized to recombinantly produce CG55069 proteins. A nucleic acid sequence encoding a CG55069 protein can be inserted into an expression vector for propagation and expression in host cells.

An expression construct, as used herein, refers to a nucleic acid sequence encoding a CG55069 protein operably associated with one or more regulatory regions that enable expression of a CG55069 protein in an appropriate host cell. “Operably-associated” refers to an association in which the regulatory regions and the CG55069 sequence to be expressed are joined and positioned in such a way as to permit transcription, and ultimately, translation.

The regulatory regions that are necessary for transcription of CG55069 can be provided by the expression vector. A translation initiation codon (ATG) may also be provided if a CG55069 gene sequence lacking its cognate initiation codon is to be expressed. In a compatible host-construct system, cellular transcriptional factors, such as RNA polymerase, will bind to the regulatory regions on the expression construct to effect transcription of the modified CG55069 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CMT sequence, and the like. The non-coding region 3′ to the coding sequence may contain transcriptional termination regulatory sequences, such as terminators and polyadenylation sites.

In order to attach DNA sequences with regulatory functions, such as promoters, to a CG55069 gene sequence or to insert a CG55069 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG55069 sequence operably associated with regulatory regions can be directly introduced into appropriate host cells for expression and production of a CG55069 protein without further cloning. See, e.g., U.S. Pat. No. 5,580,859. The expression constructs can also contain DNA sequences that facilitate integration of a CG55069 sequence into the genome of the host cell, e.g., via homologous recombination. In this instance, it is not necessary to employ an expression vector comprising a replication origin suitable for appropriate host cells in order to propagate and express CG55069 in the host cells.

A variety of expression vectors may be used, including but are not limited to, plasmids, cosmids, phage, phagemids or modified viruses. Such host-expression systems represent vehicles by which the coding sequences of a CG55069 gene may be produced and subsequently purified, but also represent cells which may, when transformed or transfected with the appropriate nucleotide coding sequences, express CG55069 in situ. These include, but are not limited to, microorganisms such as bacteria (e.g., E. coli and B. subtilis) transformed with recombinant bacteriophage DNA, plasmid DNA or cosmid DNA expression vectors containing CG55069 coding sequences; yeast (e.g., Saccharomyces, Pichia) transformed with recombinant yeast expression vectors containing CG55069 coding sequences; insect cell systems infected with recombinant virus expression vectors (e.g., baculovirus) containing CG55069 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG55069 coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter). Preferably, bacterial cells such as Escherichia coli and eukaryotic cells are used for the expression of a recombinant CG55069 molecule. For example, mammalian cells such as Chinese hamster ovary cells (CHO) can be used with a vector bearing promoter element from major intermediate early gene of cytomegalovirus for effective expression of a CG55069 sequence (Foecking et al., 1986, Gene 45:101; and Cockett et al., 1990, Bio/Technology 8:2).

In bacterial systems, a number of expression vectors may be advantageously selected depending upon the use intended for the CG55069 molecule being expressed. For example, when a large quantity of a CG55069 is to be produced, for the generation of pharmaceutical compositions of a CG55069 molecule, vectors that direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows one to cleave off the CG55069 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG55069 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG55069 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG55069 in infected hosts (see, e.g., Logan & Shenk, 1984, Proc. Natl. Acad. Sci. USA 8 1:355-359). Specific initiation signals may also be required for efficient translation of inserted CG55069 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells. Expression in a bacterial or yeast system can be used if post-translational modifications are found to be non-essential for a desired activity of CG55069. In a preferred embodiment, E. coli is used to express a CG55069 sequence.

For long-term, high-yield production of properly processed CG55069, stable expression in cells is preferred. Cell lines that stably express CG55069 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG55069 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11 (5):155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk−, hgprt− or aprt− cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG55069. Modified culture conditions and media may also be used to enhance production of CG55069. Any techniques known in the art may be applied to establish the optimal conditions for producing CG55069.

An alternative to producing CG55069 or a fragment thereof by recombinant techniques is peptide synthesis. For example, an entire CG55069, or a protein corresponding to a portion of CG55069, can be synthesized by use of a peptide synthesizer. Conventional peptide synthesis or other synthetic protocols well known in the art may be used.

Proteins having the amino acid sequence of CG55069 or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The proteins are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG55069 protein is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG55069 proteins can be found herein.

### Characterization and Demonstration of CG55069 Activities

Any methods known in the art can be used to determine the identity of a purified CG55069 protein in a composition used in accordance to the instant invention. Such methods include, but are not limited to, Western Blot, sequencing (e.g., Edman sequencing), liquid chromatography (e.g., HPLC, RP-HPLC with both UV and electrospray mass spectrometric detection), mass spectrometry, total amino acid analysis, peptide mapping, and SDS-PAGE. The secondary, tertiary and/or quaternary structure of a CG55069 protein can analyzed by any methods known in the art, e.g., far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure

The purity of a CG55069 protein in a composition used in accordance to the instant invention can be analyzed by any methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), and Western Blot (e.g., host cell protein Western Blot). In a preferred embodiment, a CG55069 protein in a composition used in accordance to the instant invention is at least 97%, at least 98%, or at least 99% pure by densitometry. In another preferred embodiment, a CG55069 protein in a composition used in accordance to the instant invention is more than 97%, more than 98%, or more than 99% pure by densitometry.

The biological activities and/or potency of CG55069 used in accordance with the present invention can be determined by any methods known in the art. For example, compositions for use in therapy in accordance to the methods of the present invention can be tested in suitable cell lines for one or more activities that Ten-M3 possesses (e.g., antiangiogenic, inhibition of cell migration activity). Non-limiting examples of such assays are described herein.

Compositions for use in a therapy in accordance to the methods of the present invention can also be tested in suitable animal model systems prior to testing in humans. Such animal model systems include, but are not limited to, mucositis models in rats, mice, hamsters, chicken, cows, monkeys, rabbits, etc. The principle animal models for mucositis known in the art include, but are not limited to, mice oral mucositis model, Xu et al., Radiother Oncol 1:369-374 (1984); hamster oral mucositis model, Sonis, In: Teicher B (ed) Tumor models in cancer research, Humana Press, Totowa, N.J. (2002); rat gastrointestinal mucositis model, Gibson et al., J Gastroenterol Hepato 18:1095-1100 (2003); mouse intestinal stem cells, Potten et al., Gut 36(6):864-873 (1995).

To establish an estimate of drug activity in model experiments, an index can be developed that combines observational examination of the animals as well as their survival status. Any staging/scoring system for human patients known in the art may also be used to evaluate the effectiveness of the compositions of the invention. Further, any assays known to those skilled in the art can be used to evaluate the prophylactic and/or therapeutic utilities of the combinatorial therapies disclosed herein. The effectiveness of CG55069 on preventing and/or treating disease can be monitored by any methods known to one skilled in the art.

Prophylactic and Therapeutic Uses

The present invention provides methods of preventing angiogenesis and/or cell migration and therefore preventing and/or treating cancer comprising administering to a subject in need thereof an effective amount of a composition comprising one or more isolated CG55069 proteins or an antibody thereto.

Malignant conditions that can be prevented and/or treated by the methods of the invention includes, but is not limited to, neuroblastoma, renal carcinoma, fibrosarcoma, rhabdosarcoma, glioblastoma, lung cancer or pancreatic cancer. In some embodiments, the methods of the invention comprise administering an effective amount of a composition comprising one or more isolated CG55069 proteins to a subject. In some embodiments, the methods of the invention comprise administering an effective amount of a composition comprising an antibody to CG55069 to a subject. The present invention provides methods of preventing angiogenesis and/or cell migration and therefore preventing and/or treating cancer in patient populations with or at risk to develop such cancers.

In accordance to the instant invention, a composition comprising one or more isolated CG55069 proteins or antibodies thereto can also be used in combination with other therapies to prevent and/or treat disease. In one embodiment, a composition comprising one or more isolated CG55069 proteins is administered in combination with one or more other agents that have prophylactic and/or therapeutic effect(s) on preventing angiogenesis and/or cell migration and therefore preventing and/or treating cancer.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more G53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, or at least 10 mg/kg (as measured by UV assay). In another embodiment, the dosage of a composition comprising one or more CG55069 proteins for administration in a human patient provided by the present invention is between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG55069 protein (instead of IgG). Test results obtained with this UV method (using CG55069 reference standard) are three times lower than test results for the same sample(s) tested with the Bradford method (using IgG as calibrator). For example, if a dosage of a composition comprising one or more CG55069 proteins for administration in a human patient provided by the present invention is between 0.001-10 mg/kg measured by UV assay, then the dosage is 0.003-30 mg/kg as measured by Bradford assay.

Pharmaceutical Compositions

The compositions used in accordance to the present invention can be administered to a subject at a prophylactically or therapeutically effective amount to prevent angiogenesis and/or cell migration and therefore preventing and/or treating cancer. Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG55069 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) that are most sensitive In some embodiments, where the composition of the invention is a nucleic acid encoding a prophylactic or therapeutic agent, the nucleic acid can be administered in vivo to promote expression of their encoded proteins (e.g., CG55069 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG55069, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG55069 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG55069 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG55069 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG55069 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG55069 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, micelles, emulsions, sphingomyelins, lipid-protein or lipid-peptide complexes, fatty acids, fatty acid esters, steroids, chelating agents and surfactants. The compositions comprising CG55069 may be encapsulated within liposomes or may form complexes thereto, in particular to cationic liposomes. Alternatively, the compositions comprising CG55069 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG55069 can be formulated in an aerosol form, spray, mist or in the form of drops or powder if intranasal administration is preferred. In particular, a composition comprising CG55069 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG55069 proteins may also be formulated into a microcapsule with one or more polymers (e.g., hydroxyethyl starch) form the surface of the microcapsule. Such formulations have benefits such as slow-release.

A composition comprising CG55069 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG55069 can be delivered to a subject by pulmonary administration, e.g., by use of an inhaler or nebulizer, of a composition formulated with an aerosolizing agent.

In a preferred embodiment, a composition comprising CG55069 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG55069 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG55069 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one ore more CG55069 proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG55069 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more CG55069 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG55069 proteins.

The invention also provides kits for carrying out the therapeutic regimens of the invention. Such kits comprise in one or more containers prophylactically or therapeutically effective amounts of the composition of the invention (e.g., a composition comprising one or more CG55069 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG55069 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

Anti-CG55069 Antibodies

Included in the invention are antibodies to CG55069 protein, or a fragment, derivative, fragment, analog, homolog or ortholog thereof. Such antibodies include, but are not limited to, immunoglobulin molecules and immunologically active portions of immunoglobulin (Ig) molecules, i.e., molecules that contain an antigen binding site that specifically binds (immunoreacts with) an antigen, polyclonal, monoclonal, chimeric, single chain, F_{ab}, F_{ab′} and F_{(ab′)2} fragments, and an F_{ab} expression library. Antibodies may be of the classes IgG, IgM, IgA, IgE and IgD, and include subclasses such as IgG₁, IgG₂, and others. The light chain may be a kappa chain or a lambda chain. Reference herein to antibodies includes a reference to all such classes, subclasses and types of antibody species.

CG55069 full length protein or a portion or fragment thereof, can be used as an immunogen to generate antibodies that immunospecifically bind the antigen, using standard techniques for polyclonal and monoclonal antibody preparation. An antigenic peptide fragment comprises at least 6 amino acid residues of the amino acid sequence of the full length protein, and encompasses an epitope. The antigenic peptide may comprise at least 10 amino acid residues, or at least 15, at least 20,, or at least 30 amino acid residues. Epitopes of the antigenic peptide are commonly regions of the protein that are located on its surface; often these are hydrophilic regions.

In certain embodiments of the invention, at least one epitope encompassed by the antigenic peptide is a region of CG55069 that is located on the surface of the protein, e.g., a hydrophilic region and can be determined by a hydrophobicity analysis of the protein sequence. As a means for targeting antibody production, hydropathy plots showing regions of hydrophilicity and hydrophobicity can be generated by any method well known in the art (for example see Proc. Nat. Acad. Sci. USA 78: 3824-3828, 1981; J. Mol. Biol. 157:105-142, 1982).

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An anti-CG55069 antibody of the present invention is said to specifically bind to CG55069 when the equilibrium binding constant (K_{D}) is ≦1 μM, preferably ≦100 nM, more preferably ≦10 nM, and most preferably ≦100 pM to about 1 pM, as measured by assays including radioligand binding assays or similar assays known to skilled artisans.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y.).

In another embodiment CG55069 nucleic acid molecules are used directly for production of antibodies recognizing CG55069 polypeptides. Antibodies can be prepared by genetic or DNA-based immunization. It has been shown that intramuscular immunization of mice with a naked DNA plasmid led to expression of reporter proteins in muscle cells (Science 247: 1465-1468, 1990) and that this technology could stimulate an immune response (Nature. 356: 152-154, 1992). The success of genetic immunization in stimulating both cellular and humoral immune responses has been widely reported (reviewed in: Annu. Rev. Immunol. 15: 617-648, 1997; Immunol. Today 19: 89-97, 1998; Annu. Rev. Immunol. 18: 927-974, 2000). Using this technology, antibodies can be generated through immunization with a cDNA sequence encoding the protein in question. Following genetic immunization, the animal's immune system is activated in response to the synthesis of the foreign protein. The quantity of protein produced in vivo following genetic immunization is within the picogram to nanogram range, which is much lower than the amounts of protein introduced by conventional immunization protocols. Despite these low levels of protein, a very efficient immune response is achieved due to the foreign protein being expressed directly in, or is quickly taken up by antigen-presenting dendritic cells (J. Leuk. Biol. 66: 350-356, 1999; J. Exp. Med. 186: 1481-1486, 1997; Nat. Med. 2: 1122-1128, 1996). A further increase in the effectivity of genetic immunization is due to the inherent immune-enhancing properties of the DNA itself, i.e., the presence of CpG-motifs in the plasmid backbone, which activate both dendritic cells (J. Immunol. 161: 3042-3049, 1998) and B-cells (Nature 374: 546-549, 1995). Genetic immunization and production of high affinity monoclonal antibodies has been successful in mice (Biotechniques 16: 616-620, 1994; J. Biotechnol. 51: 191-194, 1996; Hybridoma 17: 569-576, 1998; J. Virol. 72: 4541-4545, 1998; J. Immunol. 160: 1458-1465, 1998; J. Biotechnol. 73:119-129, 1999). It has been shown that monoclonal antibodies of the mature IgG subclasses can be obtained (Hybridoma 17: 569-576, 1998) and single chain libraries can be generated from genetically immunized mice (Proc. Natl. Acad. Sci. USA 95: 669-674, 1998). It has also been shown that genetic immunization can generate antibodies in other species such as rabbits (J. Lipid. Res. 38: 2627-2632, 1997) and turkeys (J. Lipid. Res. 38: 2627-2632, 1999). Genetic immunization has been used for the production of human antibodies recognizing extracellular targets.

Anti CG55069 antibodies can further comprise humanized or human antibodies. Humanization can be performed following methods known in the art for example Nature, 321:522-525, 1986; Nature, 332:323-327, 1988; Science, 239:1534-1536, 1988; U.S. Pat. No. 5,225,539; and Curr. Op. Struct. Biol., 2:593-596, 1992.

Fully human antibodies are antibody molecules in which the entire sequence of both the light chain and the heavy chain, including the CDRs, arise from human genes. Such antibodies are termed “human antibodies”, or “fully human antibodies” herein. Human monoclonal antibodies can be prepared by methods known in the art, see Immunol Today 4: 72, 1983; In: MONOCLONAL ANTIBODIES AND CANCER THERAPY, Alan R. Liss, Inc., pp. 77-96, 1985;. Proc Natl Acad Sci USA 80: 2026-2030, 1983; In: MONOCLONAL ANTIBODIES AND CANCER THERAPY, Alan R. Liss, Inc., pp. 77-96, 1985; J. Mol. Biol., 227:381, 1991; J. Mol. Biol., 222:581, 1991; U.S. Pat. Nos. 5,545,807; 5,545,806; 5,569,825; 5,625,126; 5,633,425; 5,661,016; Bio/Technology 10, 779-783, 1992; Nature 368 856-859, 1994; Nature 368, 812-13, 1994; Nature Biotechnology 14, 845-51, 1996; Nature Biotechnology 14, 826, 1996; and Intern. Rev. Immunol. 13, 65-93, 1995; PCT publication WO 94/02602; WO 96/33735 and WO 96/34096; U.S. Pat. Nos. 5,939,598 and 5,916,771; PCT publication WO 99/53049.

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Science 246: 1275-1281, 1989) to allow rapid and effective identification of monoclonal F_{ab} fragments with the desired specificity for a protein or derivatives, fragments, analogs or homologs thereof. Antibody fragments that contain the idiotypes to a protein antigen 05/be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art, see Nature, 305:537-539, 1983 and 05/be purified by affinity chromatography steps, also see WO 93/08829; EMBO J., 10:3655-3659, 1991. For further details of generating bispecific antibodies see, for example, Methods in Enzymology, 121:210 (1986); WO 96/27011; Science 229:81 (1985); J. Exp. Med. 175:217-225 (1992) J. Immunol. 148(5): 1547-1553 (1992); “diabody” technology described in Proc. Natl. Acad. Sci. USA 90:6444-6448 (1993); and single-chain Fv (sFv) dimers in J. Immunol. 152:5368 (1994). Antibodies with more than two valencies are contemplated, see for example J. Immunol. 147:60 (1991).

Heteroconjugate antibodies composed of two covalently joined antibodies are also within the scope of the present invention, see for example, U.S. Pat. No. 4,676,980; WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, see for example, J. Exp Med., 176: 1191-1195, 1992; J. Immunol., 148: 2918-2922, 1992; Cancer Research, 53: 2560-2565, 1993; Anti-Cancer Drug Design, 3: 219-230, 1989.

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate). Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹ In, ⁹⁰Y, and ¹⁸⁶Re. Conjugates of the antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described Science, 238:1098, 1987. Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

The antibodies disclosed herein can also be formulated as immunoliposomes prepared by methods known in the art, such as described in PNAS USA, 82: 3688, 1985; PNAS USA, 77: 4030, 1980; and U.S. Pat. Nos. 4,485,045; 4,544,545; and 5,013,556; J. Biol. Chem., 257: 286-288, 1982; J. National Cancer Inst., 81(19): 1484, 1989.

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of CG55069 protein is facilitated by generation of hybridomas that bind to the fragment of CG55069 protein possessing such a domain. Thus, antibodies that are specific for a desired domain within CG55069 protein, or derivatives, fragments, analogs or homologs thereof, are also provided herein.

Antibodies directed against CG55069 protein of the invention may be used in methods known within the art relating to the localization and/or quantitation of CG55069 protein (e.g., for use in measuring levels of CG55069 protein within appropriate physiological samples, for use in diagnostic methods, for use in imaging the protein, and the like). In a given embodiment, antibodies specific to CG55069 protein, or derivative, fragment, analog or homolog thereof, that contain the antibody derived antigen binding domain, are utilized as pharmacologically active compounds (referred to hereinafter as “Therapeutics”).

An antibody specific for CG55069 protein of the invention (e.g., a monoclonal antibody or a polyclonal antibody) can be used to isolate CG55069 polypeptide by standard techniques, such as immunoaffinity, chromatography or immunoprecipitation. An antibody to CG55069 polypeptide can facilitate the purification of a natural CG55069 antigen from cells, or of a recombinantly produced CG55069 antigen expressed in host cells. Moreover, such an anti-CG55069 antibody can be used to detect the antigenic CG55069 protein (e.g., in a cellular lysate or cell supernatant) in order to evaluate the abundance and pattern of expression of the antigenic CG55069 protein. Antibodies directed against a CG55069 protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible. Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., PNAS USA, 90: 7889-7893, 1993. The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is for example, an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

### EXAMPLES

### Example 1

### Nucleic Acid and Polypeptide Sequences of CG55069

Sequences described herein were generally derived by laboratory cloning of cDNA fragments covering the full length and/or part of the DNA sequence of the invention, and/or by in silico prediction of the full length and/or part of the DNA sequence of the invention from public human sequence databases.

TABLE 2
NUCLEIC ACID AND POLYPEPTIDE SEQUENCES OF CG55069

| | | |
|---|---|---|
| CG55069-17 | SEQ ID NO:1 | 8362 bp |
| DNA Sequence | ORF Start: ATG at 71 | ORF Stop: at 8216 |

GGCTACAGTCAGTGGAGAGGACTTTCACTGACTGACTGACTGCGTCTCAAAACCCATGGGGATCCCCA
CCATGGATGTGAAAGAACGCAGGCCTTACTGCTCCCTGACCAAGAGCAGACGAGAGAAGGAACGGCGC
TACACAAATTCCTCCGCAGACAATGAGGAGTGCCGGGTACCCACACAGAAGTCCTACAGTTCCAGCGA
GACATTGAAAGCTTTTGATCATGATTCCTCGCGGCTGCTTTACGGCAACAGAGTGAAGGATTTGGTTC
ACAGAGAAGCAGACGAGTTCACTAGACAAGGACAGAATTTTACCCTAAGGCAGTTAGGAGTTTGTGAA
CCAGCAACTCGAAGAGGACTGGCATTTTGTGCGGAAATGGGGCTCCCTCACAGAGGTTACTCTATCAG
TGCAGGGTCAGATGCTGATACTGAAAATGAAGCAGTGATGTCCCCAGAGCATGCCATGAGACTTTGGG
GCAGGGGGGTCAAATCAGGCCGCAGCTCCTGCCTGTCAAGTCGGTCCAACTCAGCCCTCACCCTGACA
GATACGGAGCACGAAAACAAGTCCGACAGTGAGAATGAGCAACCTGCAAGCAATCAAGGCCAGTCTAC
CCTGCAGCCCTTGCCGCCTTCCCATAAGCAGCACTCTGCACAGCATCATCCATCCATCACTTCTCTCA
ACAGAAACTCCCTGACCAATAGAAGGAACCAGAGTCCGGCCCCGCCGGCTGCTTTGCCCGCCGAGCTG
CAAACCACACCCGAGTCCGTCCAGCTGCAGGACAGCTGGGTCCTTGGCAGTAATGTACCACTGGAAAG
CAGGCATTTCCTATTCAAAACAGGAACAGGTACAACGCCACTGTTCAGTACTGCAACCCCAGGATACA
CAATGGCATCTGGCTCTGTTTATTCACCACCTACTCGGCCACTACCTAGAAACACCCTATCAAGAAGT
GCTTTTAAATTCAAGAAGTCTTCAAAGTACTGTAGCTGGAAATGCACTGCACTGTGTGCCGTAGGGGT
CTCGGTGCTCCTGGCAATACTCCTGTCTTATTTTATAGCAATGCATCTCTTTGGCCTCAACTGGCAGC
TACAGCAGACTGAAAATGACACATTTGAGAATGGAAAAGTGAATTCTGATACCATGCCAACAAACACT
GTGTCATTACCTTCTGGAGACAATGGAAAATTAGGTGGATTTACGCAAGAAAATAACACCATAGATTC
CGGAGAACTTGATATTGGCCGAAGAGCAATTCAAGAGATTCCTCCCGGGATCTTCTGGAGATCACAGC
TCTTCATTGATCAGCCACAGTTTCTTAAATTCAATATCTCTCTTCAGAAGGATGCATTGATTGGAGTA
TATGGCCGGAAAGGCTTACCGCCTTCCCATACTCAGTATGACTTCGTGGAGCTCCTGGATGGCAGCAG
GCTGATTGCCAGAGAGCAGCGGAGCCTGCTTGAGACGGAGAGAGCCGGGCGGCAGGCGAGATCCGTCA
GCCTTCATGAGGCCGGCTTTATCCAGTACTTGGATTCTGGAATCTGGCATCTGGCTTTTTATAATGAT
GGGAAAAATGCAGAGCAGGTGTCTTTTAATACCATTGTTATAGAGTCTGTGGTGGAATGTCCCCGAAA
TTGCCATGGAAATGGAGAATGCGTTTCTGGAACTTGCCATTGTTTTCCAGGATTTCTGGGTCCGGATT
GTTCAAGAGCCGCCTGTCCAGTGTTATGTAGTGGCAACGGGCAGTACTCCAAGGGCCGCTGCCTGTGT
TTCAGCGGCTGGAAGGGCACCGAGTGTGATGTGCCGACTACCCAGTGTATTGACCCACAGTGTGGGGG
TCGTGGGATTTGTATCATGGGCTCTTGTGCTTGCAACTCAGGATACAAAGGAGAAAGTTGTGAAGAAG
CTGACTGTATAGACCCTGGGTGTCATAATCATGGTGTGTGTATCCACGGGGAATGTCACTGCAGTCCA
GGATGGGGAGGTAGCAATTGTGAAATACTGAAGACCATGTGTCCAGACCAGTGCTCCGGCCACGGAAC
GTATCTTCAAGAAAGTGGCTCCTGCACGTGTGACCCTAACTGGACTGGCCCAGACTGCTCAAACGAAA
TATGTTCTGTGGACTGTGGCTCACACGGCGTTTGCATGGGGGGGACGTGTCGCTGTGAAGAAGGCTGG
ACGGGCCCAGCCTGTAATCAGAGAGCCTGCCACCCCCGCTGTGCCGAGCACGGGACCTGCAAGGATGG
CAAGTGTGAATGCAGCCAGGGCTGGAATGGAGAGCACTGCACTATCGCTCACTATTTGGATAAGATAG
TTAAAGAGGGTTGTCCTGGTCTGTGCAACAGCAATGGAAGATGTACCCTGGACCAAAATGGCTGGCAT
TGTGTGTGCCAGCCTGGATGGAGAGGAGCAGGCTGTGACGTAGCCATGGAGACTCTTTGCACAGATAG
CAAGGACAATGAAGGAGATGGACTCATTGACTGCATGGACCCCGATTGCTGCCTACAGAGTTCCTGCC
AGAATCAGCCCTATTGTCGGGGACTGCCGGACCCTCAGGACATCATTAGCCAAAGCCTTCAATCGCCT
TCTCAGCAAGCTGCCAAATCCTTTTATGATCGAATCAGTTTCCTTATAGGATCTGATAGCACCCATGT
TATACCTGGAGAAAGTCCTTTCAATAAGAGCCTTGCATCTGTCATCAGAGGCCAAGTACTGACTGCTG
ATGGAACTCCACTTATTGGAGTAAATGTcTCGTTTTTCCATTACCCAGAATATGGATATACTATTACC
CGCCAGGACGGAATGTTTGACTTGGTGGCAAATGGTGGGGCCTCTCTAACTTTGGTATTTGAACGATC
CCCATTCCTCACTCAGTATCATACTGTGTGGATTCCATGGAATGTCTTTTATGTGATGGATACCCTAG
TCATGAAGAAAGAAGAGAATGATATTCCCAGCTGTGATCTGAGTGGATTCGTGAGGCCAAATCCCATC
ATTGTGTCATCACCTTTATCCACCTTTTTCAGATCTTCTCCTGAAGACAGTCCCATCATTCCCGAAAC
ACAGGTACTCCACGAGGAAACTACAATTCCAGGAACAGATTTGAAACTCTCCTACTTGAGTTCCAGAG
CTGCAGGGTATAAGTCAGTTCTCAAGATCACCATGACCCAGTCTATTATTCCATTTAATTTAATGAAG
GTTCATCTTATGGTAGCTGTAGTAGGAAGACTCTTCCAAAAGTGGTTTCCTGCCTCACCAAACTTGGC
CTATACTTTCATATGGGATAAAACAGATGCATATAATCAGAAAGTCTATGGTCTATCTGAAGCTGTTG
TGTCAGTTGGATATGAGTATGAGTCGTGTTTGGACCTGACTCTGTGGGAAAAGAGGACTGCCATTCTG
CAGGGCTATGAATTGGATGCGTCCAACATGGGTGGCTGGACATTAGATAAACATCACGTGCTGGATGT
ACAGAACGGTATACTGTACAAGGGAAACGGGGAAAACCAGTTCATCTCCCAGCAGCCTCCAGTCGTGA
GTAGCATCATGGGCAATGGGCGAAGGCGCAGCATTTCCTGCCCCAGTTGCAATGGTCAAGCTGATGGT
AACAAGTTACTGGCCCCAGTGGCGCTAGCTTGTGGGATCGATGGCAGTCTGTACGTAGGCGATTTCAA
CTATGTGCGGCGGATATTCCCTTCTGGAAATGTAACAAGTGTCTTAGAACTAAGAAATAAAGATTTTA
GACATAGCAGCAACCCAGCTCATAGATACTACCTTGCAACGGACCCAGTCACGGGAGATCTGTACGTT
TCTGACACAAACACCCGCAGAATTTATCGCCCAAAGTCACTTACGGGGGCAAAAGACTTGACTAAAAA
TGCAGAAGTCGTCGCAGGGACAGGGGAGCAATGCCTTCCGTTTGACGAGGCGAGATGTGGGGATGGAG
GGAAGGCCGTGGAAGCCACACTCATGAGTCCCAAAGGAATGGCAGTTGATAAGAATGGATTAATCTAC
TTTGTTGATGGAACCATGATTAGGAAAGTTGACCAAAATGGAATCATATCAACTCTTCTGGGCTCTAA
CGATTTGACTTCAGCCAGACCTTTAACTTGTGACACCAGCATGCACATCAGCCAGGTACGTCTGGAAT
GGCCCACTGACCTAGCCATTAACCCTATGGATAACTCCATTTATGTCCTGGATAATAATGTAGTTTTA
CAGATCACTGAAAATCGTCAAGTTCGCATTGCTGCTGGACGGCCCATGCACTGTCAGGTTCCCGGAGT
GGAATATCCTGTGGGGAAGCACGCGGTGCAGACAACACTGGAATCAGCCACTGCCNTTGCTGTGTCCT
ACAGTGGG3TCCTGTACATTACTGAAACTGATGAGAAGAAAATTAACCGGATAAGGCAGGTCACAACA
GATGGAGAAATCTCCTTAGTGGCCGGAATACCTTCAGAGTGTGACTGCAAAAATGATGCCAACTGTGA
CTGTTACCAGAGTGGAGATGGCTACGCCAAGGATGCCAAACTCAGTGCCCCATCCTCCCTGGCTGCTT
CTCCAGATGGTACACTGTATATTGCAGATCTAGGGAATATCCGGATACGGGCTGTGTCAAAGAATAAG
CCTTTACTTAACTCTATGAACTTCTATGAAGTTGCGTCTCCAACTGATCAAGAACTCTACATCTTTGA
CATCAATGGTACTCACCAATATACTGTAAGTTTAGTCACTGGTGATTACCTTTACAATTTTAGCTACA
GCAATGACAATGATATTACTGCTGTGACAGACAGCAATGGCAACACCCTTAGAATTAGACGGGACCCA
AATCGCATGCCAGTTCGAGTGGTGTCTCCTGATAACCAAGTGATATGGTTGACAATAGGAACAAATGG
ATGTTTGAAAAGCATGACTGCTCAAGGACTGGAATTAGTTTTGTTTACTTACCATGGCAATAGTGGCC
TTTTAGCCACTAAAAGTGATGAAACTGGATGGACAACGTTTTTTGACTATGACAGTGAAGGTCGTCTG
ACAAATGTTACGTTTCCAACTGGAGTGGTCACAAACCTGCATGGGGACATGGACAAGGCTATCACAGT
GGACATTGAGTCATCTAGCCGAGAAGAAGATGTCAGCATCACTTCAAATCTGTCCTCGATCGATTCTT
TCTACACCATGGTTCAAGATCAGTTAAGAAACAGCTACCAGATTGGTTATGACGGCTCCCTCAGAATT
ATCTACGCCAGTGGCCTGGACTCACACTACCAAACAGAGCCGCACGTTCTGGCTGGCACCGCTAATCC
GACGGTTGCCAAAAGAAACATGACTTTGCCTGGCGAGAACGGTCAAAACTTGGTGGAATGGAGATTCC
GAAAAGAGCAAGCCCAAGGGAAAGTCAATGTCTETGGCCGCAAGCTCAGGGTTAATGGCAGAAACCTC
CTTTCAGTTGACTTTGATCGAACAACAAAGACAGAAAAGATCTATGACGACCACCGTAAATTTCTACT
GAGGATCGCCTACGACACGTCTGGGCACCCGACTCTCTGGCTGCCAAGCAGCAAGCTGATGGCCGTCA
ATGTCACCTATTCATCCACAGGTCAAATTGCCAGCATCCAGCGAGGCACCACTAGCGAGAAAGTAGAT
TATGACGGACAGGGGAGGATCGTGTCTCGGGTCTTTGCTGATGGTAAAACATGGAGTTACACATATTT
AGAAAAGTCCATGGTTCTTCTGCTTCATAGCCAGCGGCAGTACATCTTCGAATACGATATGTGGGACC
GCCTGTCTGCCATCACCATGCCCAGTGTGGCTCGCCACACCATGCAGACCATCCGATCCAYTGGCTAC
TACCGCAACATATACAACCCCCCGGAAAGCAACGCCTCCATCATCACGGACTACAACGAGGAAGGGCT
GCTTCTACAAACAGCTTTCTTGGGTACAAGTCGGAGGGTCTTATTCAAATACAGAAGGCAGACTAGGC
TCTCAGAAATTTTATATGATAGCACAAGAGTCAGTTTTACCTATGATGAAACAGCAGGAGTCCTAAAG
ACAGTAAACCTCCAGAGTGATGGTTTTATTTGCACCATTAGATACAGGCAAATTGGTCCCCTGATTGA
CAGGCAGATTTTCCGGTTTAGTGAAGATGGGATGGTAAATGCAAGATTTGACTATAGCTATGACAACA
GCTTTCGAGTGACCAGCATGCAGGGTGTGATCAATGAAACGCCACTGCCTATTGATCTGTATCAGTTT
GATGACATTTCTGGCAAAGTTGAGCAGTTTGGAAAGTTTGGAGTTATATATTATGATATTAACCAGAT
CATTTCTACAGCTGTAATGACCTATACGAAGCACTTTGATGCTCATGGCCGTATCAAGGAGATTCAAT
ATGAGATATTCAGGTCGCTCATGTACTGGATTACAATTCAGTATGATAACATGGGTCGGGTAACCAAG
AGAGAGATTAAAATAGGGCCCTTTGCCAACACCACCAAATATGCTTATGAATATGATGTTGATGGACA
GCTCCAAACAGTTTACCTCAATGAAAAGATAATGTGGCGGTACAACTACGATCTGAATGGAAACCTCC
ATTTACTGAACCCAAGTAACAGTGCGCGTCTGACACCCCTTCGCTATGACCTGCGAGACAGAATCACT
CGACTGGGTGATGTTCAATATCGGTTGGATGAAGATGGTTTCCTACGTCAAAGGGGCACGGAAATCTT
TGAATATAGCTCCAAGGGGCTTCTAACTCGCGTTTACAGTAAAGGCAGTGGCTGGACAGTGATCTACC
GTTATGACGGCCTGGGAAGGCGTGTTTCTAGCAAAACCAGTCTAGGACAGCACCTGCAGTTTTTTTAT
GCTGACTTAACTTATCCCACTAGGATTACTCATGTCTACAACCATTCGAGTTCAGAAATTACCTCCCT
GTATTATGATCTCCAAGGACATCTTTTTGCCATGGAAATCAGCAGTGGGGATGAATTCTATATTGCAT
CGGATAACACAGGGACACCACTGGCTGTGTTCAGTAGCAATGGGCTTATGCTGAAACAGATTCAGTAC
ACTGCATATGGGGAAATCTATTTTGACTCTAATATTGACTTTCAACTGGTAATTGGATTTCATGGTGG
CCTGTATGACCCACTCACCAAATTAATCCACTTTGGAGAAAGAGATTATGACATTTTGGCAGGACGGT
GGACAACACCTGACATAGAAATCTGGAAAAGAATTGGGAAGGACCCAGCTCCTTTTAACTTGTACATG
TTTAGGAATAACAACCCTGCAAGCAAAATCCATGACGTGAAAGATTACATCACAGATGTTAACAGCTG
GCTGGTGACATTTGGTTTCCATCTGCACAATGCTATTCCTGGATTCCCTGTTCCCAAATTTGATTTAA
CAGAACCTTCTTACGAACTTGTGAAGAGTCAGCAGTGGGATGATATACCGCCCATCTTCGGAGTCCAG
CAGCAAGTGGCGCGGCAGGCCAAGGCCTTCCTGTCGCTGGGGAAGATGGCCGAGGTGCAGGTGAGCCG
GCGCCGGGCCGGCGGCGCGCAGTCCTGGCTGTGGTTCGCCACGGTCAAGTCGCTGATCGGCAAGGGCG
TCATGCTGGCCGTCAGCCAGGGCCGCGTGCAGACCAACGTGCTCAACATCGCCAACGAGGACTGCATC
AAGGTGGCGGCCGTGCTCAACAACGCCTTCTACCTGGAGAACCTGCACTTCACCATCGAGGGCAAGGA
CACGCACTACTTCATCAAGACCACCACGCCCGAGAGCGACCTGGGCACGCTGCGGTTGACCAGCGGCC
GCAAGGCGCTGGAGAACGGCATCAACGTGACGGTGTCGCAGTCCACCACGGTGGTGAACGGCAGGACG
CGCAGGTTCGCGGACGTGGAGATGCAGTTCGGCGCGCTGGCGCTGCACGTGCGCTACGGCATGACCCT
GGACGAGGAGAAGGCGCGCATCCTGGAGCAGGCGCGGCAGCGCGCGCTCGCCCGGGCCTGGGCGCGCG
AGCAGCAGCGCGTGCGCGACGGCGAGGAGGGCGCGCGCCTCTGGACGGAGGGCGAGAAGCGGCAGCTG
CTGAGCGCCGGCAAGGTGCAGGGCTACGACGGGTACTACGTACTCTCGGTGGAGCAGTACCCCGAGCT
GGCCGACAGCGCCAACAACATCCAGTTCCTGCGGCAGAGCGAGATCGGCAGGAGGGGTAAGCCTATCC
CTAACCCTCTCCTCGGTCTCGATTCTACGCGTACCGGTCATCATCACCATCACCATTAACTCGAGCTC
GAGTGGTCAGTCTTCACTGACTGACTGACTGGAAAGAGGAAGGGCTGGAAGAGGAAGGAGCTTGGC

| | | | |
|---|---|---|---|
| CG55069-17 | SEQ ID NO:2 | 2715 aa | MW at 302969.6kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTG1TPLFSTATPGYT
MASGSVYSPPTRPLPRNTLSRSAFKFKKSSKYCSWKCTALCAVGVSVLLAILLSYFIAMHLFGLNWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKGLPPSHTQYDFVELLDGSRLIAREQRSLLETERAGRQARSVS
LHEAGFIQYLDSGIWHLAFYNDGKNAEQVSFNTIVIESVVECPRNCHGNGECVSGTCHCFPGFLGPDC
SRAACPVLCSGNGQYSKGRCLCFSGWKGTECDVPTTQCIDPQCGGRGICLMGSCACNSGYKGESCEEA
DCIDPGCSNHGVCIHGECHCSPGWGGSNCEILKTMCPDQCSGHGTYLQESGSCTCDPNWTGPDCSNEI
CSVDCGSHGVCMGGTCRCEEGWTGPACNQRACHPRCAEHGTCKDGKCECSQGWNGEHCTIAHYLDKIV
KEGCPGLCNSNGRCTLDQNGWHCVCQPGWRGAGCDVAMETLCTDSKDNEGDGLIDCMDPDCCLQSSCQ
NQPYCRGLPDPQDIISQSLQSPSQQAAKSFYDRJSFLIGSDSTHVIPGESPFNKSLASVIRGQVLTAD
GTPLIGVNVSFFHYPEYGYTITRQDGMFDLVANGGASLTLVFERSPFLTQYHTVWLPWNVFYVMDTLV
MKKEENDIPSCDLSGFVRPNPIIVSSPLSTFFRSSPEDSPILPETQVLHEETTIPGTDLKLSYLSSRA
AGYKSVLKITMTQSIIPFNLMKVHLMVAVVGRLFQKWFPASPNLAYTFIWDKTDAYNQKVYGLSEAVV
SVGYEYESCLDLTLWEKRTAILQGYELDASNMGGWTLDKHHVLDVQNGILYKGNGENQFISQQPPVVS
SIMGNGRRRSISCPSCNGQADGNKLLAPVALACGIDGSLYVGDFNYVRRIFPSGNVTSVLELRNKDFR
HSSNPAHRYYLATDPVTGDLYVSDTNTRRIYRPKSLTGAKDLTKNAEVVAGTGEQCLPFDEARCGDGG
KAVEATLMSPKGMAVDKNGLIYFVDGTMJRKVDQNGIISTLLGSNDLTSARPLTCDTSMHISQVRLEW
PTDLAINPMDNSIYVLDNNVVLQITENRQVRIAAGRPMHCQVPGVEYPVGKHAVQULESATAIIAVSY
SGVLYITETDEKKINRIRQVTTDGEISLVAGIPSECDCKNDANCDCYQSGDGYAKDAKLSAPSSLAAS
PDGTLYIADLGNIRIRAVSKNKPLLNSMNFYEVASPTDQELYLFDINGTHQYTVSLVTGDYLYNFSYS
NDNDITAVTDSNGNTLRIRRDPNRMPVRVVSPDNQVIWLTIGTNGCLKSMTAQGLELVLFTYHGNSGL
LATKSDETGWTTFFDYDSEGRLTNVTFPTGVVTNLHGDMDKAITVDIESSSREEDVSITSNLSSIDSF
YTMVQDQLRNSYQIGYDGSLRIIYASGLDSHYQTEPHVLAGTANPTVAKRNMTLPGENGQNLVEWRFR
KEQAQGKVNVFGRKLRVNGRNLLSVDFDRTTKTEKIYDDHRKFLLRIAYDTSGHPTLWLPSSKLMAVN
VTYSSTGQIASIQRGTTSEKVTYDGQGRIVSRVFADGKTWSYTYLEKSMVLLLHSQRQYIFEYDMWDR
LSAITMPSVARHTMQTIRSIGYYRNIYNPPESNASIITDYNEEGLLLQTAFLGTSRRVLFKYRRQTRL
SEILYDSTRVSFTYDETAGVLKTVNLQSDGFICTIRYRQIGPLIDRQIFRFSEDGMVNARFDYSYDNS
FRVTSMQGVINETPLPIDLYQFDDISGKVEQFGKFGVIYYDINQIISTAVMTYTKHFDAHGRIKEIQY
EIFRSLMYWITIQYDNMGRVTKREIKIGPFANTTKYAYEYDVDGQLQTVYLNEKTMWRYNYDLNGNLH
LLNPSNSARLTPLRYDLRDRITRLGDVQYRLDEDGFLRQRGTELFEYSSKGLLTRVYSKGSGWTVIYR
YDGLGRRVSSKTSLGQHLQFFYADLTYPTRITHVYNHSSSEITSLYYDLQGHLFAMEISSGDEFYIAS
DNTGTPLAVFSSNGLMLKQIQYTAYGEIYFDSNIDFQLVIGFHGGLYDPLTKLIHFGERDYDILAGRW
TTPDIEJWKRIGKDPAPFNLYMFRNNNPASKIHDVKDYITDVNSWLVTFGFHLHNAIPGFPVPKFDLT
EPSYELVKSQQWDDIPPIFGVQQQVARQAKAFLSLGKMAEVQVSRRRAGGAQSWLWFATVKSLIGKGV
MLAVSQGRVQTNVLNIANEDCIKVAAVLNNAFYLENLHFTLEGKDTHYFIKTITPESDLGTLRLTSGR
KALENGINVTVSQSTTVVNGRTRRFADVEMQFGALALHVRYGMTLDEEKARILEQARQRALARAWARE
QQRVRDGEEGARLWTEGEKRQLLSAGKVQGYDGYYVLSVEQYPELADSANNIQFLRQSEIGRR

| | | |
|---|---|---|
| CG55069-16 | SEQ ID NO:3 | 7786bp |
| DNA Sequence | ORF Start: at 476 | ORF Stop: at 7604 |

AACAGTGGAGGCCAGACTTAGGCACAGCACGATGCCCACCACCACCAGTGTGCCGCACAAGGCCGTGG
CGGTAGGGTATGTGTCTGAAAATGAGCTCGGGGAGCGGGCTTGCACCGCTGACGCATTTGGAAGACTT
AAGGCAGCGGCAGAAGAAGATGCAGGCAGCTGAGTTGTTGTGTTCTGATAAGAGTCAGAGGTAACTCC
CGTTGCGGTGCTGTTAACGGTGGAGGGCAGTGTAGTCTGAGCAGTACTCGTTGCTGCCGCGCGCGCCA
CCAGACATAATAGCTGACAGACTAACAGACTGTTCCTTTCCATGGGTCTTTTCTGCAGTCACCGTCCT
TGACACGAAGCTCTAGCCACCATGGAGACAGACACACTCCTGCTATGGGTACTGCTGCTCTGGGTTCC
AGGTTCCACTGGTGACGCGGCCCAGCCGGCCAGGCGCGCGCGCCGTACGAAGCTTTCGCGAGGATCCC
TACAGCAGACTGAAAATGACACATTTGAGAATGGAAAAGTGAATTCTGATACCATGCCAACAAACACT
GTGTCATTACCTTCTGGAGACAATGGAAAATTAGGTGGATTTACGCAAGAAAATAACACCATAGATTC
CGGAGAACYTGATATTGGCCGAAGAGCAATTCAAGAGATTCCTCCCGGGATCTTCTGGAGATCACAGC
TCTTCATTGATCAGCCACAGTETCTTAAATTCAATATCTCTCTTCAGAAGGATGCATTGATTGGAGTA
TATGGCCGGAAAGGCTTACCGCCTTCCCATACTCAGTATGACTTCGTGGAGCTCCTGGATGGCAGCAG
GCTGATTGCCAGAGAGCAGCGGAGCCTGCTTGAGACGGAGAGAGCCGGGCGGCAGGCGAGATCCGTCA
GCCTTCATGAGGCCGGCTTTATCCAGTACTTGGATTCTGGAATCTGGCATCTGGCTTTTTATAATGAT
GGGAAAAATGCAGAGCAGGTGTCTTTTAATACCATTGTTATAGAGTCTGTGGTGGAATGTCCCCGAAA
TTGCCATGGAAATGGAGAATGCGTTTCTGGAACTTGCCATTGTTTTCCAGGATTTCTGGGTCCGGATT
GTTCAAGAGCCGCCTGTCCAGTGTTATGTAGTGGCAACGGGCAGTACTCCAAGGGCCGCTGCCTGTGT
TTCAGCGGCTGGAAGGGCACCGAGTGTGATGTGCCGACTACCCAGTGTATTGACCCACAGTGTGGGGG
TCGTGGGATETGTATCATGGGCTCTTGTGCTTGCAACTCAGGATACAAAGGAGAAAGTTGTGAAGAAG
CTGACTGTATAGACCCTGGGTGTTCTAATCATGGTGTGTGTATCCACGGGGAATGTCACTGCAGTCCA
GGATGGGGAGGTAGCAATTGTGAAATACTGAAGACCATGTGTCCAGACCAGTGCTCCGGCCACGGAAC
GTATCTTCAAGAAAGTGGCTCCTGCACGTGTGACCCTAACTGGACTGGCCCAGACTGCTCAAACGAAA
TATGTTCTGTGGACTGTGGCTCACACGGCGTTTGCATGGGGGGGACGTGTCGCTGTGAAGAAGGCTGG
ACGGGCCCAGCCTGTAATCAGAGAGCCTGCCACCCCCGCTGTGCCGAGCACGGGACCTGCAAGGATGG
CAAGTGTGAATGCAGCCAGGGCTGGAATGGAGAGCACTGCACTATCGCTCACTATTTGGATAAGATAG
TTAAAGAGGGTTGTCCTGGTCTGTGCAACAGCAATGGAAGATGTACCCTGGACCAAAATGGCTGGCAT
TGTGTGTGCCAGCCTGGATGGAGAGGAGCAGGCTGTGACGTAGCCATGGAGACTCTTTGCACAGATAG
CAAGGACAATGAAGGAGATGGACTCATTGACTGCATGGACCCCGATTGCTGCCTACAGAGTTCCTGCC
AGAATCAGCCCTATTGTCGGGGACTGCCGGACCCTCAGGACATCATTAGCCAAAGCCTTCAATCGCCT
TCTCAGCAAGCTGCCAAATCCTTTTATGATCGAATCAGTTTCCTTATAGGATCTGATAGCACCCATGT
TATACCTGGAGAAAGTCCTTTCAATAAGAGCCTTGCATCTGTCATCAGAGGCCAAGTACTGACTGCTG
ATGGAACTCCACTTATTGGAGTAAATGTCTCGTTTTTCCATTACCCAGAATATGGATATACTATTACC
CGCCAGGACGGAATGTTTGACTTGGTGGCAAATGGTGGGGCCTCTCTAACTTTGGTATTTGAACGATC
CCCATTCCTCACTCAGTATCATACTGTGTGGATTCCATGGAATGTGTTTTATGTGATGGATACCCTAG
TCATGAAGAAAGAAGAGAATGATATTCCCAGCTGTGATCTGAGTGGATTCGTGAGGCCAAATCCCATC
ATTGTGTCATCACCTTTATCCACCTTTTTCAGATCTTCTCCTGAAGACAGTCCCATCATTCCCGAAAC
ACAGGTACTCCACGAGGAAACTACAATTCCAGGAACAGATTTGAAACTCTCCTACTTGAGTTCCAGAG
CTGCAGGGTATAAGTCAGTTCTCAAGATCACCATGACCCAGTCTATTATTCCATTTAATTTAATGAAG
GTTCATCTTATGGTAGCTGTAGTAGGAAGACTCTTCCAAAAGTGGTTTCCTGCCTCACCAAACTTGGC
CTATACTTTCATATGGGATAAAACAGATGCATATAATCAGAAAGTCTATGGTCTATCTGAAGCTGTTG
TGTCAGTTGGATATGAGTATGAGTCGTGTTTGGACCTGACTCTGTGGGAAAAGAGGACTGCCATTCTG
CAGGGCTATGAATTGGATGCGTCCAACATGGGTGGCTGGACATTAGATAAACATCACGTGCTGGATGT
ACAGAACGGTATACTGTACAAGGGAAACGGGGAAAACCAGTTCATCTCCCAGCAGCCTCCAGTCGTGA
GTAGCATCATGGGCAATGGGCGAAGGCGCAGCATTTCCTGCCCCAGTTGCAATGGTCAAGCTGATGGT
AACAAGTTACTGGCCCCAGTCGCGCTAGCTTGTGGGATCGATGGCAGTCTGTACGTAGGCGATTTCAA
CTATGTGCGGCGGATATTCCCTTCTGGAAATGTAACAAGTGTCTTAGAACTAAGAAATAAAGATTTTA
GACATAGCAGCAACCCAGCTCATAGATACTACCTTGCAACGGACCCAGTCACGGGAGATCTGTACGTT
TCTGACACAAACACCCGCAGAATTTATCGCCCAAAGTCACTTACGGGGGCAAAAGACTTGACTAAAAA
TGCAGAAGTCGTCGCAGGGACAGGGGAGCAATGCCTTCCGTLTGACGAGGCGAGATGTGGGGATGGAG
GGAAGGCCGTGGAAGCCACACTCATGAGTCCCAAAGGAATGGCAGTTGATAAGAATGGATTAATCTAC
TTTGTTGATGGAACCATGATTAGGAAAGTTGACCAAAATGGAATCATATCAACTCTTCTGGGCTCTAA
CGATTTGACTTCAGCCAGACCTTTAACTTGTGACACCAGCATGCACATCAGCCAGGTACGTCTGGAAT
GGCCCACTGACCTAGCCATTAACCCTATGGATAACTCCATTTATGTCCTGGATAATAATGTAGTTTTA
CAGATCACTGAAAATCGTCAAGTTCGCATTGCTGCTGGACGGCCCATGCACTGTCAGGTTCCCGGAGT
GGAATATCCTGTGGGGAAGCACGCGGTGCAGACAACACTGGAATCAGCCACTGCCATTGCTGTGTCCT
ACAGTGGGGTCCTGTACATTACTGAAACTGATGAGAAGAAAATTAACCGGATAAGGCAGGTCACAACA
GATGGAGAAATCTCCTTAGTGGCCGGAATACCTTCAGAGTGTGACTGCAAAAATGATGCCAACTGTGA
CTGTTACCAGAGTGGAGATGGCTACGCCAAGGATGCCAAACTCAGTGCCCCATCCTCCCTGGCTGCTT
CTCCAGATGGTACACTGTATATTGCAGATCTAGGGAATATCCGGATACGGGCTGTGTCAAAGAATAAG
CCTTTACTTAACTCTATGAACTTCTATGAAGTTGCGTCTCCAACTGATCAAGAACTCTACATCTTTGA
CATCAATGGTACTCACCAATATACTGTAA&TTTAGTCACTGGTGATTACCTTTACAATTTTAGCTACA
GCAATGACAATGATATTACTGCTGTGACAGACAGCAATGGCAACACCCTTAGAATTAGACGGGACCCA
AATCGCATGCCAGTTCGAGTGGTGTCTCCTGATAACCAAGTGATATGGTTGACAATAGGAACAAATGG
ATGTTTGAAAAGCATGACTGCTCAAGGACTGGAATTAGTTTTGTTTACTTACCATGGCAATAGTGGCC
TTTTAGCCACTAAAAGTGATGAAACTGGATGGACAACGTTTTTTGACTATGACAGTGAAGGTCGTCTG
ACAAATGTTACGTTTCCAACTGGAGTGGTCACAAACCTGCATGGGGACATGGACAAGGCTATCACAGT
GGACATTGAGTCATCTAGCCGAGAAGAAGATGTCAGCATCACTTCAAATCTGTCCTCGATCGATTCTT
TCTACACCATGGTTCAAGATCAGTTAAGAAACAGCTACCAGATTGGTTATGACGGCTCCCTCAGANTT
ATCTACGCCAGTGGCCTGGACTCACACTACCAAACAGAGCCGCACGTTCTGGCTGGCACCGCTAATCC
GACGGTTGCCAAAAGAAACATGACTTTGCCTGGCGAGAACGGTCAAAACTTGGTGGAATGGAGATTCC
GAAAAGAGCAAGCCCAAGGGAAAGTCAATGTCTTTGGCCGCAAGCTCAGGGTTAATGGCAGAAACCTC
CTTTCAGTTGACTTTGATCGAACAACAAAGACAGAAAAGATCTATGACGACCACCGTAAATTTCTACT
GAGGATCGCCTACGACACGTCTGGGCACCCGACTCTCTGGCTGCCAAGCAGCAAGCTGATGGCCGTCA
ATGTCACCTATTCATCCACAGGTCAAATTGCCAGCATCCAGCGAGGCACCACTAGCGAGAAAGTAGAT
TATGACGGACAGGGGAGGATCGTGTCTCGGGTCTTTGCTGATGGTAAAACATGGAGTTACACATATTT
AGAAAAGTCCATGGTTCTTCTGCTTCATAGCCAGCGGCAGTACATCTTCGAATACGATATGTGGGACC
GCCTGTCTGCCATCACCATGCCCAGTGTGGCTCGCCACACCATGCAGACCATCCGATCCATTGGCTAC
TACCGCAACATATACAACCCCCCGGAAAGCAACGCCTCCATCATCACGGACTACAACGAGGAAGGGCT
GCTTCTACAAACAGCTTTCTTGGGTACAAGTCGGAGGGTCTTATTCAAATACAGAAGGCAGACTAGGC
TCTCAGAAATTTTATATGATAGCACAAGAGTCAGTTTTACCTATGATGAAACAGCAGGAGTCCTAAAG
ACAGTAAACCTCCAGAGTGATGGTETTATTTGCACCATTAGATACAGGCAAATTGGTCCCCTGATTGA
CAGGCAGATTTTCCGCTTTAGTGAAGATGGGATGGTAAATGCAAGATTTGACTATAGCTATGACAACA
GCTTTCGAGTGACCAGCATGCAGGGTGTGATCAATGAAACGCCACTGCCTATTGATCTGTATCAGTTT
GATGACATTTCTGGCAAAGTTGAGCAGTTTGGAAAGTTTGGAGTTATATATTATGATATTAACCAGAT
CATTTCTACAGCTGTAATGACCTATACGAAGCACTTTGATGCTCATGGCCGTATCAAGGAGATTCAAT
ATGAGATATTCAGGTCGCTCATGTACTGGATTACAATTCAGTATGATAACATGGGTCGGGTAACCAAG
AGAGAGATTAAAATAGGGCCCTTTGCCAACACCACCAAATATGCTTATGAATATGATGTTGATGGACA
GCTCCAAACAGTTTACCTCAATGAAAAGATAATGTGGCGGTACAACTACGATCTGAATGGAAACCTCC
ATTTACTGAACCCAAGTAACAGTGCGCGTCTGACACCCCTTCGCTATGACCTGCGAGACAGAATCACT
CGACTGGGTGATGTTCAATATCGGTTGGATGAAGATGGTTTCCTACGTCAAAGGGGCACGGAAATCTT
TGAATATAGCTCCAAGGGGCTTCTAACTCGCGTTTACAGTAAAGGCAGTGGCTGGACAGTGATCTACC
GTTATGACGGCCTGGGAAGGCGTGTTTCTAGCAAAACCAGTCTAGGACAGCACCTGCAGTTTTTTTAT
GCTGACTTAACTTATCCCACTAGGATTACTCATGTCTACAACCATTCGAGTTCAGAAATTACCTCCCT
GTATTATGATCTCCAAGGACATCTTTTTGCCATGGAAATCAGCAGTGGGGATGAATTCTATATTGCAT
CGGATAACACAGGGACACCACTGGCTGTGTTCAGTAGCAATGGGCTTATGCTGAAACAGATTCAGTAC
ACTGCATATGGGGAAATCTATTTTGACTCTAATATTGACTTTCAACTGGTAATTGGATTTCATGGTGG
CCTGTATGACCCACTCACCAAATTAATCCACTTTGGAGAAAGAGATTATGACATTTTGGCAGGACGGT
GGACAACACCTGACATAGAAATCTGGAAAAGAATTGGGAAGGACCCAGCTCCTTTTAACTTGTACATG
TTTAGGAATAACAACCCTGCAAGCAAAATCCATGACGTGAAAGATTACATCACAGATGTTAACAGCTG
GCTGGTGACATTGGTTTTCCATCTGCACAATGCTATTCCTGGATTCCCTGTTCCCAAATTTGATTTAA
CAGAACCTTCTTACGAACTTGTGAAGAGTCAGCAGTGGGATGATATACCGCCCATCTTCGGAGTCCAG
CAGCAAGTGGCGCGGCAGGCCAAGGCCTTCCTGTCGCTGGGGAAGATGGCCGAGGTGCAGGTGAGCCG
GCGCCGGGCCGGCGGCGCGCAGTCCTGGCTGTGGTTCGCCACGGTCAAGTCGCTGATCGGCAAGGGCG
TCATGCTGGCCGTCAGCCAGGGCCGCGTGCAGACCAACGTGCTCAACATCGCCAACGAGGACTGCATC
AAGGTGGCGGCCGTGCTCAACAACGCCTTCTACCTGGAGAACCTGCACTTCACCATCGAGGGCAAGGA
CACGCACTACTTCATCAAGACCACCACGCCCGAGAGCGACCTGGGCACGCTGCGGTTGACCAGCGGCC
GCAAGGCGCTGGAGAACGGCATCAACGTGACGGTGTCGCAGTCCACCACGGTGGTGAACGGCAGGACG
CGCAGGTTCGCGGACGTGGAGATGCAGTTCGGCGCGCTGGCGCTGCACGTGCGCTACGGCATGACCCT
GGACGAGGAGAAGGCGCGCATCCTGGAGCAGGCGCGGCAGCGCGCGCTCGCCCGGGCCTGGGCGCGCG
AGCAGCAGCGCGTGCGCGACGGCGAGGAGGGCGCGCGCCTCTGGACGGAGGGCGAGAAGCGGCAGCTG
CTGAGCGCCGGCAAGGTGCAGGGCTACGACGGGTACTACGTACTCTCGGTGGAGCAGTACCCCGAGCT
GGCCGACAGCGCCAACAACATCCAGTTCCTGCGGCAGAGCGAGATCGGCAGGAGGGGTAAGCCTATCC
CTAACCCTCTCCTCGGTCTCGATTCTACGCGTACCGGTCATCATCACCATCACCATTAACTCGAGGGT
AAGCCTATCCCTAACCCTCTCCTCGGTCTCGATTCTACGCGTACCGGTCATCACCACCATCACCATTG
AGTTTAATTCATGATCATATCAGCCATACACATT

| | | | |
|---|---|---|---|
| CG55069-16 | SEQ ID NO:4 | 2376 aa | MW at 265358.9kD |
| Protein Sequence | | | |

LQQTENDTFENGKVNSDTMPTNTVSLPSGDNGKLGGFTQENNTIDSGELDIGRRAIQEIPPGIFWRSQ
LFIDQPQFLKFNISLQKDALIGVYGRKGLPPSHTQYDFVELLDGSRLIAREQRSLLETERAGRQARSV
SLHEAGFIQYLDSG1WHLAFYNDGKNAEQVSFNTIVIESVVECPRNCHGNGECVSGTCHCFPGFLGPD
CSRAACPVLCSGNGQYSKGRCLCFSGWKGTECDVPTTQCIDPQCGGRGICIMGSCACNSGYKGESCEE
ADCIDPGCSNHGVCIHGECHCSPGWGGSNCEILKTMCPDQCSGHGTYLQESGSCTCDPNWTGPDCSNE
ICSVDCGSHGVCMGGTCRCEEGWTGPACNQRACHPRCAEHGTCKDGKCECSQGWNGEHCTIAHYLDKI
VKEGCPGLCNSNGRCTLDQNGWHCVCQPGWRGAGCDVAMEThCTDSKDNEGDGLIDCMDPDCCLQSSC
QNQPYCRGLPDPQDIISQSLQSPSQQAAKSFYDRISFLIGSDSTHVIPGESPFNKSLASVIRGQVLTA
DGTPLIGVNVSFFHYPEYGYTITRQDGMFDLVANGGASLTLVFERSPFLTQYHTVWIPWNVFYVMDTL
VMKKEENDIPSCDLSGFVRPNPIIVSSPLSTFFRSSPEDSPILPETQVLHEE1TLPGTDLKLSYLSSR
AAGYKSVLKITMTQSIIPFNLMKVHLMVAVVGRLFQKWFPASPNLAYTFIWDKTDAYNQKVYGLSEAV
VSVGYEYESCLDLTLWEKRTAILQGYELDASNMGGWTLDKHHVLDVQNGILYKGNGENQFISQQPPVV
SSLMGNGRRRSISCPSCNGQADGNKLLAPVALACGIDGSLYVGDFNYVRRIFPSGNVTSVLELRNKDF
RHSSNPAHRYYLATDPVTGDLYVSDTNTRRIYRPKSLTGAKDLTKNAEVVAGTGEQCLPFDEARCGDG
GKAVEATLMSPKGMAVDKNGLIYFVDGTMIRKVDQNGIISTLLGSNDLTSARPLTCDTSMHISQVRLE
WPTDLAINPMDNSIYVLDNNVVLQITENRQVRIAAGRPMHCQVPGVEYPVGKHAVQTTLESATAIAVS
YSGVLYITETDEKKINRIRQVTTDGEISLVAGIPSECDCKNDANCDCYQSGDGYAKDAKLSAPSSLAA
SPDGTLYIADLGNIRIRAVSKNKPLLNSMNFYEVASPTDQELYLFDINGTHQYTVSLVTGDYLYNFSY
SNDNDITAVTDSNGNTLRIRRDPNRMPVRVVSPDNQVIWLTIGTNGCLKSMTAQGLELVLFTYHGNSG
LLATKSDETGWTTFFDYDSEGRLTNVTFPTGVVTNLHGDMDKAITVDIESSSREEDVSITSNLSSIDS
FYTMVQDQLRNSYQIGYDGSLRIIYASGLDSHYQTEPHVLAGTANPTVAKRNMTLPGENGQNLVEWRF
RKEQAQGKVNVFGRKLRVNGRNLLSVDFDRTrKTEKIYDDHRKFLLRIAYDTSGHPTLWLPSSKLMAV
NVTYSSTGQIASIQRGITSEKVDYDGQGRIVSRVFADGKTWSYTYLEKSMVLLLHSQRQYIFEYDMWD
RLSAITMPSVARHTMQTIRSIGYYRNIYNPPESNASIITDYNEEGLLLQTAFLGTSRRVLFKYRRQTR
SLEILYDSTRVSFTYDETAGVLKTVNLQSDGFICTIRYRQIGPLIDRQIFRFSEDGMVNARFDYSYDN
SFRVTSMQGVINETPLPIDLYQFDDISGKVEQFGKFGVIYYDINQIISTAVMTYTKHFDAHGRIKEIQ
YEIFRSLMYWITIQYDNMGRVTKREIKIGPFANTIKYAYEYDVDGQLQTVYLNEKIMWRYNYDLNGNL
HLLNPSNSARLTPLRYDLRDRITRLGDVQYRLDEDGFLRQRGTEIFEYSSKGLLTRVYSKGSGWTVIY
RYDGLGRRVSSKTSLGQHLQFFYADLTYPTRITHVYNHSSSEITSLYYDLQGHLFAMEISSGDEFYIA
SDNTGTPLAVFSSNGLMLKQIQYTAYGEIYFDSNIDFQLVIGFHGGLYDPLTKLIHFGERDYDILAGR
WTTPDIEIWKRJGKDPAPFNLYMFRNNNPASKJHDVKDYITDVNSWLVTFGFHLHNAJPGFPVPKFDL
TEPSYELVKSQQWDDLPPIFGVQQQVARQAKAFLSLGKMAEVQVSRRRAGGAQSWLWFATVKSLIGKG
VMLAVSQGRVQTNVLNIANEDCIKVAAVLNNAFYLENLHVfIEGKDTHYFIKTITPESDLGTLRLTSG
RKALENGINVTVSQSTTVVNGRTRRFADVEMQFGALALHVRYGMTLDEEKARILEQARQRALARAWAR
EQQRVRDGEEGARLWTEGEKRQLLSAGKVQGYDGYYVLSVEQYPELADSANNIQFLRQSEIGRR

| | | |
|---|---|---|
| CG55069-11 | SEQ ID NO:5 | 2482 bp |
| DNA Sequence | ORF Start: at 11 | ORF Stop: at 2474 |

CACCTCGCGAAACTGGCAGCTACAGCAGACTGAAAATGACACATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGGCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGGCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCAYTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGGGCACCGAGTGTGATGTGCCGACTACCCAGTGT
ATTGACCCACAGTGTGGGGGTCGTGGGATTTGTATCATGGGCTCTTGTGCETGCAACTCAGGATACAA
AGGAGAAAGTT3TGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGT3CTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GTCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGAGGGTTGTCCTGGTCTGTGCAACAGCAATGGAAGATGTACCC
TGGACCAAAATGGCTGGCATTGTGTGTGCCAGCCTGGATGGAGAGGAGCAGGCTGTGACGTAGCCATG
GAGACTCTTTGCACAGATAGTAAGGACAATGAAGGAGATGGACTCATTGACTGCATGGATCCCGATTG
CTGCCTACAGAGTTCCTGCCAGAATCAGCCCTATTGTCGGGGACTGCCGGATCCTCAGGACATCATTA
GCCAAAGCCTTCAATCGCCTTCTCAGCAAGCTGCCAAATCCTTTTATGATCGAATCAGTTTCCTTATA
GGATCTGATAGCACCCATGTTATACCTGGAGAAAGTCCTTTCAATAAGAGCCTTGCATCTGTCATCAG
AGGCCAAGTACTGACTGCTGATGGAACTCCACTTATTGGAGTAAATGTCTCGTTTTTCCATTACCCAG
AATATGGATATACTATTACCCGCCAGGACGGAATGTTTGACTTGGTGGCAAATGGTGGGGCCTCTCTA
ACTTTGGTATTTGAACGATCCCCATTCCTCACTCAGTATCATACTGTGTGGATTCCATGGAATGTCTT
TTATGTGATGGATACCCTAGTCATGAAGAAAGAAGAGAATGACATTCCCAGCTGTGATCTGAGTGGAT
TCGTGAGGCCAAATCCCATCATTGTGTCATCACCTTTATCCACCTTTTTCAGATCTTCTCCTGAAGAC
AGTCCCATCATTCCCGAAACACAGGTACTCCACGAGGAAACTACAATTCCAGGAACAGAYTTGAAACT
CTCCTACTTGAGTTCCAGAGCTGCAGGGTATAAGTCAGTTCTCAAGATCACCATGACCCAGTCTATTA
TTCCATTTAATTTAATGAAGGTTCATCTTATGGTAGCTGTAGTAGGAAGACTCTTCCAAAAGTGGTTT
CCTGCCTCACCAAACTTGGCCTATACTTTCATATGGGATAAAACAGATGCATATAATCAGAAAGTCTA
TGGTCTATCTGAAGCTGTTGTGTCAGYTGGATATGAGTATGAGTCGTGTTTGGACCTGACTCTGTGGG
AAAAGAGGACTGCCATTCTGCAGGGCTATGAATTGGATGCGTCCAACATGGGTGGCTGGACATTAGAT
AAACATCACGTGCTGGATGTACAGAACGGTATACTGTACAAGGGAAACGGGGAAAACCAGTTCATCTC
CCAGCAGCCTCCAGTCGTGAGTAGCCTCGAGGGC

| | | | |
|---|---|---|---|
| CG55069-11 | SEQ ID NO:6 | 821 aa | MW at 89886.1kD |
| Protein Sequence | | | |

NWQLQQTENDTFENGKVNSDTMPTNTVSLPSGDNGKLGGFTQENNTIDSGELDIGRRAIQEIPPGIFW
RSQLFIDQPQFLKFNISLQKDALIGVYGRKGLPPSHTQYDFVELLDGSRLIAREQRSLLETERAGRQA
RSVSLHEAGFIQYLDSGIWHLAFYNDGKNAEQVSFNTIVLESVVECPRNCHGNGECVSGTCHCFPGFL
GPDCSRAACPVLCSGNGQYSKGRCLCFSGWKGTECDVPTTQCIDPQCGGRGICIMGSCACNSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKEGCPGLCNSNGRCTLDQNGWHCVCQPGWRGAGCDVAMETLCTDSKDNEGDGLIDCMDPDCCLQ
SSCQNQPYCRGLPDPQDIISQSLQSPSQQAAKSFYDRISFLIGSDSTHVIPGESPFNKSLASVIRGQV
LTADGTPLIGVNVSFFHYPEYGYTITRQDGMFDLVANGGASLTLVFERSPFLTQYHTVWLPWNVFYVM
DTLVMKKEENDIPSCDLSGFVRPNPIIVSSPLSTFFRSSPEDSPIIIETQVLHEETfLPGTDLKLSYL
SSRAAGYKSVLKITMTQSIJPFNLMKVHLMVAVVGRLFQKWFPASPNLAYTFLWDKTDAYNQKVYGLS
EAVVSVGYEYESCLDLTLWEKRTAILQGYELDASNMGGWTLDKHHVLDVQNGILYKGNGENQFISQQP
PVVSS

| | | |
|---|---|---|
| CG55069-01 | SEQ ID NO:7 | 8657 bp |
| DNA Sequence | ORF Start: ATG at 151 | ORF Stop: TAA at 8326 |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCTTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAGAAGTTACCGCCTTCCCATACTCAGTCCTCCCCCCAGTATGACTT
CGTGGAGCTCCTGGATGGCAGCAGGCTGATTGCCAGAGAGCAGCGGAGCCTGCTTGAGACGGAGAGAG
CCGGGCGGCAGGCGAGATCCGTCAGCCTTCATGAGGCCGGCTTTATCCAGTACTTGGAYTCTGGAATC
TGGCATCTGGCTTTTTATAATGATGGGAAAAATGCAGAGCAGGTGTCTTTTAATACCATTGTTATAGA
GTCTGTGGTGGAATGTCCCCGAAATTGCCATGGAAATGGAGAATGCGTTTCTGGAACTTGCCATTGTT
TTCCAGGATTTCTGGGTCCGGATTGTTCAAGAGCCGCCTGTCCAGTGTTATGTAGTGGCAACGGGCAG
TACTCCAAGGGCCGCTGCCTGTGTTTCAGCGGCTGGAAGGGCACCGAGTGTGATGTGCCGACTACCCA
GTGTATTGACCCACAGTGTGGGGGTCGTGGGATTTGTATCATGGGCTCCTGTGCTTGCAGCTCAGGAT
ACAAAGGAGAAAGTTGTGAAGAAGCTGACTGTATAGACCCTGGGTGTTCTAATCATGGTGTGTGTATC
CACGGGGAATGTCACTGCAGTCCAGGATGGGGAGGTAGCAATTGTGAAATACTGAAGACCATGTGTCC
AGACCAGTGCTCCGGCCACGGAACGTATCTTCAAGAAAGTGGCTCCTGCACGTGTGACCCTAACTGGA
CTGGCCCAGACTGCTCAAACGAAATATGTCTGTGGACTGTGGCTCACACGGCGTTTTGCATGGGGGGG
ACGTGTCGCTGTGAAGAAGGCTGGACGGGCCCAGCCTGTAATCAGAGAGCCTGCCACCCCCGCTGTGC
CGAGCACGGGACCTGCAAGGATGGCAAGTGTGAATGCAGCCAGGGCTGGAATGGAGAGCACTGCACTA
TCGCTCACTATTTGGATAAGATAGTTAAAGACAAGATAGGATATAAAGAGGGTTGTCCTGGTCTGTGC
AACAGCAATGGAAGATGTACCCTGGACCAAAATGGCGGACATTGTGTGTGCCAGCCTGGATGGAGAGG
AGCAGGCTGTGACGTAGCCATGGAGACTCTTTGCACAGATAGCAAGGACAATGAAGGGGATGGACTCA
TTGACTGCATGGATCCCGATTGCTGCCTACAGAGTTCCTGCCAGAATCAGCCCTATTGTCGGGGACTG
CCGGATCCTCAGGACATCATTAGCCAAAGCCTTCAATCGCCTTCTCAGCAAGCTGCCAAATCCTTTTA
TGATCGAATCAGTTTCCTTATAGGATCTGATAGCACCCATGTGAGACCTGGAGAAAGTCCTTTCAATA
AGAGCCTTGCATCTGTCATCAGAGGCCAAGTACTGACTGCTGATGGAACTCCACTTATTGGAGTAAAT
GTCTCGTTTTTCCATTACCCAGAATATGGATATACTATTACCCGCCAGGACGGAATGTTTGACTTGGT
GGCAAATGGTGGGGCCTCTCTAACTTTGGTATTTGAACGATCCCCATTCCTCACTCAGTATCATACTG
TGTGGATTCCATGGAATGTCTTTTATGTGATGGATACCCTAGTCATGGAGAAAGAAGAGAATGACATT
CCCAGCTGTGATCTGAGTGGATTCGTGAGGCCAAATCCCATCATTGTGTCATCACCTTTATCCACCTT
TTTCAGATCTTCTCCTGAAGACAGTCCCATCATTCCCGAAACACAGGTACTCCACGAGGAAACTACAA
TTCCAGGAACAGATTTGAAACTCTCCTACTTGAGTTCCAGAGCTGCAGGGTATAAGTCAGTTCTCAAG
ATCACCATGACCCAGTCTATTATTCCATTTAATTTAATGAAGGTTCATCTTATGGTAGCTGTAGTAGG
AAGACTCTTCCAAAAGTGGTTTCCTGCCTCACCAAACTTGGCCTATACTTTCATATGGGATAAAACAG
ATGCATATAATCAGAAAGTCTATGGTCTATCTGAAGCTGTTGTGTCAGTTGGATATGAGTATGAGTCG
TGTTTGGACCTGACTCTGTGGGAAAAGAGGACTGCCATTCTGCAGGGCTATGAATTGGATGCGTCCAA
CATGGGTGGCTGGACATTAGATAAACATCACGTGCTGGATGTACAGAACGGTATACTGTACAAGGGAA
ACGGGGAAAACCAGTTCATCTCCCAGCAGCCTCCAGTCGTGAGTAGCATCATGGGCAATGGGCGAAGG
CGCAGCATTTCCTGCCCCAGTTGCAATGGTCAAGCTGATGGTAACAAGTTACTGGCCCCAGTGGCGCT
AGCTTGTGGGATCGATGGCAGTCTGTACGTAGGCGATTTCAACTACGTGCGGCGGATATTCCCTTCTG
GAAATGTAACAAGTGTCTTAGAACTAAGAAATAAAGATTTTAGACATAGCAGCAACCCAGCTCATAGA
TACTACCTTGCAACGGATCCAGTCACGGGAGATCTGTACGTTTCTGACACAAACACCCGCAGAATTTA
TCGCCCAAAGTCACTTACGGGGGCAAAAGACTTGACTAAAAATGCAGAAGTCGTCGCAGGGACAGGGG
AGCAATGCCTTCCGTTTGACGAGGCGAGATGTGGGGATGGAGGGAAGGCCGTGGAAGCCACACTCATG
AGTCCCAAAGGAATGGCAGTTGATAAGAATGGATTAATCTACTTTGTTGATGGAACCATGATTAGGAA
AGTTGACCAAAATGGAATCATATCAACTCTTCTGGGCTCTAACGATTTGACTTCAGCCAGACCTTTAA
CTTGTGACACCAGCATGCACATCAGCCAGGTACGTCTGGAATGGCCCACTGACCTACCCATTAACCCT
ATGGATAACTCCATTTATGTCCTGGATAATAATGTAGTTTTACAGATCACTGAAAATCGTCAAGTTCG
CATTGCTGCTGGACGGCCCATGCACTGTCAGGTTCCCGGAGTGGAATATCCTGTGGGGAAGCACGCGG
TGCAGACAACACTGGAATCAGCCACTGCCATTGCTGTGTCCTACAGTGGGGTCCTGTACATTACTGAA
ACTGATGAGAAGAAAATTAACCGGATAAGGCAGGTCACAACAGATGGAGAAATCTCCTTAGTGGCCGG
AATACCTTCAGAGTGTGACTGCAAAAATGATGCCAACTGTGACTGTTACCAGAGTGGAGATGGCTACG
CCAAGGATGCCAAACTCAGTGCCCCATCCTCCCTGGCTGCTTCTCCAGATGGTACACTGTATATTGCA
GATCTAGGGAATATCCGGATCCGGGCTGTGTCAAAGAATAAGCCTTTACTTAACTCTATGAACTTCTA
TGAAGTTGCGTCTCCAACTGATCAAGAACTCTACATCTTTGACATCAATGGTACTCACCAATATACTG
TAAGTTTAGTCACTGGTGATTACCTTTACAATTTTAGCTACAGCAATGACAATGATATTACTGCTGTG
ACAGACAGCAATGGCAACACCCTTAGAATTAGACGGGACCCAAATCGCATGCCAGTTCGAGTGGTGTC
TCCTGATAACCAAGTGATATGGTTGACAATAGGAACAAATGGATGTTTGAAAGGCATGACTGCTCAAG
GACTGGAATTAGTTTTGTTTACTTACCATGGCAATAGTGGCCTTTTAGCCACTAAAAGTGATGAAACT
GGATGGACAACGTTTTTTGACTATGACAGTGAAGGTCGTCTGACAAATGTTACGTTTCCAACTGGAGT
GGTCACAAACCTGCATGGGGACATGGACAAGGCTATCACAGTGGACATTGAGTCATCTAGCCGAGAAG
AAGATGTCAGCATCACTTCAAATCTGTCCTCGATCGATTCTTTcTACACCATGGTTCAAGATCAGTTA
AGAAACAGCTACCAGATTGGTTATGACGGCTCCCTCAGAATTATCTACGCCAGTGGCCTGGACTCACA
CTACCAAACAGAGCCGCACGTTCTGGCTGGCACCGCTAATCCGACGGTTGCCAAAAGAAACATGACTT
TGCCTGGCGAGAACGGTCAAAACTTGGTGGAATGGAGATTCCGAAAAGAGCAAGCCCAAGGGAAAGTC
AATGTCTTTGGCCGCAAGCTCAGGGTTAATGGCAGAAACCTCCTTTCAGTTGACTTTGATCGAACAAC
AAAGACAGAAAAGATCTATGACGACCACCGTAAATTTCTACTGAGGATCGCCTACGACACGTCTGGGC
ACCCGACTCTCTGGCTGCCAAGCAGCAAGCTGATGGCCGTCAATGTCACCTATTCATCCACAGGTCAA
ATTGCCAGCATCCAGCGAGGCACCACTAGCGAGAAAGTAGATTATGACGGACAGGGGAGGATCGTGTC
TCGGGTCTTTGCTGATGGTAAAACATGGAGTTACACATATTTAGAAAAGTCCATGGTTCTTCTGCTTC
ATAGCCAGCGGCAGTACATCTTCGAATACGATATGTGGGACCGCCTGTCTGCCATCACCATGCCCAGT
GTGGCTCGCCACACCATGCAGACCATCCGATCCATTGGCTACTACCGCAACATATACAACCCCCCGGA
AAGCAACGCCTCCATCATCACGGACTACAACGAGGAAGGGCTGCTTcTACAAACAGCTTTCTTGGGTA
CAAGTCGGAGGGTCTTATTCAAATACAGAAGGCAGACTAGGCTCTCAGAAATTTTATATGATAGCACA
AGAGTCAGTTTTACCTATGATGAAACAGCAGGAGTCCTAAAGACAGTAAACCTCCAGAGTGATGGTTT
TATTTGCACCATTAGATACAGGCAAATTGGTCCCCTGATTGACAGGCAGATTTTCCGCTTTAGTGAAG
ATGGGATGGTAAATGCAAGATTTGACTATAGCTATGACAACAGCTTTCGAGTGACCAGCATGCAGGGT
GTGATCAATGAAACGCCACTGCCTATTGATCTGTATCAGTTTGATGACATTTCTGGCAAAGTTGAGCA
GTTTGGAAAGTTTGGAGTTATATATTATGATATTAACCAGATCATTTCTACAGCTGTAATGACCTATA
CGAAGCACTTTGATGCTCATGGCCGTATCAAGGAGATTCAATATGAGATATTCAGGTCGCTCATGTAC
TGGATTACAATTCAGTATGATAACATGGGTCGGGTAACCAAGAGAGAGATTAAAATAGGGCCCTTTGC
CAACACCACCAAATATGCTTATGAATATGATGTTGATGGACAGCTCCAAACAGTTTACCTCAATGAAA
AGATAATGTGGCGGTACAACTACGATCTGAATGGAAACCTCCATTTACTGAACCCAAGTAACAGTGCG
CGTCTGACACCCCTTCGCTATGACCTGCGAGACAGAATCACTCGACTGGGTGATGTTCAATATCGGTT
GGATGAAGATGGTTTCCTACGTCAAAGGGGCACGGAAATCTTTGAATATAGCTCCAAGGGGCTTCTAA
CTCGAGTTTACAGTAAAGGCAGTGGCTGGACAGTGATCTACCGTTATGACGGCCTGGGAAGGCGTGTT
TCTAGCAAAACCAGTCTAGGACAGCACCTGCAGTTTTTTTATGCTGACTTAACTTATCCCACTAGGAT
TACTCATGTCTACAACCATTCGAGTTCAGAAATTACCTCCCTGTATTATGATCTCCAAGGACATCTTT
TTGCCATGGAAATCAGCAGTGGGGATGAATTCTATATTGCATCGGATAACACAGGGACACCACTGGCT
GTGTTCAGTAGCAATGGGCTTATGCTGAAACAGATTCAGTACACTGCATATGGGGAAATCTATTTTGA
CTCTAATATTGACTTTCAACTGGTAATTGGATTTCATGGTGGCCTGTATGACCCACTCACCAAATTAA
TCCACTTTGGAGAAAGAGATTATGACATTTTGGCAGGACGGTGGACAACACCTGACATAGAAATCTGG
AAAAGAATTGGGAAGGACCCAGCTCCTTTTAACTTGTACATGTTTAGGAATAACAACCCTGCAAGCAA
AATCCATGACGTGAAAGATTACATCACAGATGTTAACAGCTGGCTGGTGACATTTGGTTTCCATCTGC
ACAATGCTATTCCTGGATTCCCTGTTCCCAAATTTGATTTAACAGAACCTTCTTACGAACTTGTGAAG
AGTCAGCAGTGGGATGATATACCGCCCATCTTCGGAGTCCAGCAGCAAGTGGCGCGGCAGGCCAAGGC
CTTCCTGTCGCTGGGGAAGATGGCCGAGGTGCAGGTGAGCCGGCGCCGGGCCGGCGGCGCGCAGTCCT
GGCTGTGGTTCGCCACGGTCAAGTCGCTGATCGGCAAGGGCGTCATGCTGGCCGTCAGCCAGGGCCGC
GTGCAGACCAACGTGCTCAACATCGCCAACGAGGACTGCATCAAGGTGGCGGCCGTGCTCAACAACGC
CTTCTACCTGGAGAACCTGCACTTCACCATCGAGGGCAAGGACACGCACTACTTCATCAAGACCACCA
CGCCCGAGAGCGACCTGGGCACGCTGCGGTTGACCAGCGGCCGCAAGGCGCTGGAGAACGGCATCAAC
GTGACGGTGTCGCAGTCCACCACGGTGGTGAACGGCAGGACGCGCAGGTTCGCGGACGTGGAGATGCA
GTTCGGCGCGCTGGCGCTGCACGTGCGCTACGGCATGACCCTGGACGAGGAGAAGGCGCGCATCCTGG
AGCAGGCGCGGCAGCGCGCGCTCGCCCGGGCCTGGGCGCGCGAGCAGCAGCGCGTGCGCGACGGCGAG
GAGGGCGCGCGCCTCTGGACGGAGGGCGAGAAGCGGCAGCTGCTGAGCGCCGGCAAGGTGCAGGGCTA
CGACGGGTACTACGTACTCTCGGTGGAGCAGTACCCCGAGCTGGCCGACAGCGCCAACAACATCCAGT
TCCTGCGGCAGAGCGAGATCGGCAGGAGGTAACGCCCGGGCCGCGCCCGCCGAGCCGCTCACGCCCTG
CCCACATTGTCCTGTGGCACAACCCGAGTGGGACTCTCCAACGCCCAAGAGCCTTCGTCCCGGGGGAA
TGAGACTGCTGTTACGACCCACACCCACACCGCGAAAACAAGGACCGCTTTTTTCCGAATGACCTTAA
AGGTGATCGGCTTTAACGAATATGTTTACATATGCATAGCGCTGCACTCAGTCGGACTGAACGTAGCC
AGAGGAAAAAAAAATCATCAAGGACAAAGGCCTCGACCTGTTGCGCTGGGCCGTCTGTTCCTTCTAGG
CACTGTATTTAACTAACTTTA

| | | | |
|---|---|---|---|
| CG55069-01 | SEQ ID NO:8 | 2725 aa | MW at 303959.6kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSThQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLNWQL
QQTENDTFENGKVNSDTMPTNTVSLPSGDNGKLGGFTQENNTIDSGELDIGRRAIQEIPPGIFWRSQL
FIDQPQFLKFNISLQKDALIGVYGRKKLPPSHTQSSPQYDFVELLDGSRLIAREQRSLLETERAGRQA
RSVSLHEAGFIQYLDSGIWHLAFYNDGKNAEQVSFNTIVLESVVECPRNCHGNGECVSGTCHCFPGFL
GPDCSRAACPVLCSGNGQYSKGRCLCFSGWKGTECDVPTTQCIDPQCGGRGICLMGSCACSSGYKGES
CEEADCIDPGCSNHGVCIHGECHCSPGWGGSNCEILKTMCPDQCSGHGTYLQESGSCTCDPNWTGPDC
SNEICSVDCGSHGVCMGGTCRCEEGWTGPACNQRACHPRCAEHGTCKDGKCECSQGWNGEHCTIAHYL
DKIVKDKIGYKEGCPGLCNSNGRCTLDQNGGHCVCQPGWRGAGCDVAMETLCTDSKDNEGDGLIDCMD
PDCCLQSSCQNQPYCRGLPDPQDIISQSLQSPSQQAAKSFYDRJSFLIGSDSTHVLPGESPFNKSLAS
VIRGQVLTADGTPLIGVNVSFFHYPEYGYTITRQDGMFDLVANGGASLTLVFERSPFLTQYHTVWJPW
NVFYVMDTLVMEKEENDIPSCDLSGFVRPNPIIVSSPLSTFFRSSPEDSPIIPETQVLHEETTIPGTD
LKLSYLSSRAAGYKSVLKITMTQSILPFNLMKVHLMVAVVGRLFQKWFPASPNLAYTFIWDKTDAYNQ
KVYGLSEAVVSVGYEYESCLDLTLWEKRTAILQGYELDASNMGGWTLDKHHVLDVQNGILYKGNGENQ
FISQQPPVVSSIMGNGRRRSISCPSCNGQADGNKLLAPVALACGIDGSLYVGDFNYVRRIFPSGNVTS
VLELRNKDFRHSSNPAHRYYLATDPVTGDLYVSDTNTRRIYRPKSLTGAKDLTKNAEVVAGTGEQCLP
FDEARCGDGGKAVEATLMSPKGMAVDKNGLIYFVDGTMIRKVDQNGIISTLLGSNDLTSARPLTCDTS
MHISQVRLEWPTDLAINPMDNSIYVLDNNVVLQITENRQVRIAAGRPMHCQVPGVEYPVGKHAVQTTL
ESATAIAVSYSGVLYiTETDEKKINRIRQVTTDGEISLVAGIPSECDCKNDANCDCYQSGDGYAKDAK
LSAPSSLAASPDGTLYIADLGNIRIRAVSKNKPLLNSMNFYEVASPTDQELYIFDINGTHQYTVSLVT
GDYLYNFSYSNDNDITAVTDSNGNTLRIRRDPNRMPVRVVSPDNQVIWLTIGTNGCLKGMTAQGLELV
LFTYHGNSGLLATKSDETGWITFFDYDSEGRLTNVTFPTGVVTNLHGDMDKAITVDIESSSREEDVSI
TSNLSSIDSFYTMVQDQLRNSYQIGYDGSLRIIYASGLDSHYQTEPHVLAGTANPTVAKRNMTLPGEN
GQNLVEWRFRKEQAQGKVNVFGRKLRVNGRNLLSVDFDRTTKTEKIYDDHRKFLLRIAYDTSGHPTLW
LPSSKLMAVNVTYSSTGQIASIQRGTISEKVDYDGQGRIVSRVFADGKTWSYTYLEKSMVLLLHSQRQ
YIFEYDMWDRLSAITMPSVARHTMQTIRSIGYYRNIYNPPESNASIITDYNEEGLLLQTAFLGTSRRV
LFKYRRQTRLSEILYDSTRVSFTYDETAGVLKTVNLQSDGFICTIRYRQIGPLIDRQIFRFSEDGMVN
ARFDYSYDNSFRVTSMQGVINETPLPIDLYQFDDISGKVEQFGKFGVIYYDINQIISTAVMTYTKHFD
AHGRIKEIQYEIFRSLMYWITIQYDNMGRVTKREIKIGPFANTTKYAYEYDVDGQLQTVYLNEKIMWR
YNYDLNGNLHLLNPSNSARLTPLRYDLRDRITRLGDVQYRLDEDGFLRQRGTEIFEYSSKGLLTRVYS
KGSGWTVIYRYDGLGRRVSSKTSLGQHLQFFYADLTYPTRITHVYNHSSSEITSLYYDLQGHLFAMEI
SSGDEFYIASDNTGTPLAVFSSNGLMLKQIQYTAYGEIYFDSNIDFQLVIGFHGGLYDPLTKLIHFGE
RDYDILAGRWTTPDIEIWKRIGKDPAPFNLYMFRNNNPASKIHDVKDYITDVNSWLVTFGFHLHNAJP
GFPVPKFDLTEPSYELVKSQQWDDIPPIFGVQQQVARQAKAFLSLGKMAEVQVSRRRAGGAQSWLWFA
TVKSLIGKGVMLAVSQGRVQTNVLNIANEDCIKVAAVLNNAFYLENLHFTIEGKDTHYFIKTTTPESD
LGTLRLTSGRKALENGINVTVSQSTTVVNGRTRRFADVEMQFGALALHVRYGMTLDEEKARILEQARQ
RALARAWAREQQRVRDGEEGARLWTEGEKRQLLSAGKVQGYDGYYVLSVEQYPELADSANNIQFLRQS
EIGRR

| | | |
|---|---|---|
| CG55069-02 | SEQ ID NO:9 | 18645 bp |
| DNA Sequence | ORF Start: ATG at 151 | ORF Stop: TAA at 8314 |

TTTGGCCTCGGGCCAGAATTCGGCACGAGGGGTCTGGAGCTTGGAGGAGAAGTCTGAACTAAGGATAA
ACTAAAGAGAGGCCAATGAGACTTGAACCCTGAGCCTAAGTTGTCACCAGCAGGACTGATGTGCACAC
AGAAGGAATGAAGTATGGATGTGAAAGAACGCAGGCCTTACTGCTCCCTGACCAAGAGCAGACGAGAG
AAGGAACGGCGCTACACAAATTCCTCCGCAGACAATGAGGAGTGCCGGGTACCCACACAGAAGTCCTA
CAGTTCCAGCGAGACATTGAAAGCTTTTGATCATGATTCCTCGCGGCTGCTTTACGGCAACAGAGTGA
AGGATTTGGTTCACAGAGAAGCAGACGAGTTCACTAGACAAGGACAGAATTTTACCCTAAGGCAGTTA
GGAGTTTGTGAACCAGCAACTCGAAGAGGACTGGCATTTTGTGCGGAAATGGGGCTCCCTCACAGAGG
TTACTCTATCAGTGCAGGGTCAGATGCTGATACTGAAAATGAAGCAGTGATGTCCCCAGAGCATGCCA
TGAGACTTTGGGGCAGGGGGGTCAAATCAGGCCGCAGCTCCTGCCTGTCAAGTCGGTCCAACTCAGCC
CTCACCCTGACAGATACGGAGCACGAAAACAAGTCCGACAGTGAGAATGAGCAACCTGCAAGCAATCA
AGGCCAGTCTACCCTGCAGCCCTTGCCGCCTTCCCATAAGCAGCACTCTGCACAGCATCATCCATCCA
TCACTTCTCTCAACAGAAACTCCCTGACCAATAGAAGGAACCAGAGTCCGGCCCCGCCGGCTGCTTTG
CCCGCCGAGCTGCAAACCACACCCGAGTCCGTCCAGCTGCAGGACAGCTGGGTCCTTGGCAGTAATGT
ACCACTGGAAAGCAGGCATTTCCTATTCAAAACAGGAACAGGTACAACGCCACTGTTCAGTACTGCAA
CCCCAGGATACACAATGGCATCTGGCTCTGTTTATTCACCACCTACTCGGCCACTACCTAGAAACACC
CTATCAAGAAGTGCTTTTAAATTCAAGAAGTCTTCAAAGTACTGTAGCTGGAAATGCACTGCACTGTG
TGCCGTAGGGGTCTCGGTGCTCCTGGCAATACTCCTGTCTTATTTTATAGCAATGCATCTCYTTGGCC
TCAACTGGCAGCTACAGCAGACTGAAAATGACACATTTGAGAATGGAAAAGTGAATTCTGATACCATG
CCAACAAACACTGTGTCATTACCTTCTGGAGACAATGGAAAATTAGGTGGATTTACGCAAGAAAATAA
CACCATAGATTCCGGAGAACTTGATATTGGCCGAAGAGCAATTCAAGAGATTCCTCCCGGGATCTTCT
GGAGATCACAGCTCTTCATTGATCAGCCACAGTTTCTTAAATTCAATATCTCTCTTCAGAAGGATGCA
TTGATTGGAGTATATGGCCGGAAAGGCTTACCGCCTTCCCATACTCAGTATGACTTCGTGGAGCTCCT
GGATGGCAGCAGGCTGATTGCCAGAGAGCAGCGGAGCCTGCTTGAGACGGAGAGAGCCGGGCGGCAGG
CGAGATCCGTCAGCCTTCATGAGGCCGGCTTTATCCAGTACTTGGATTCTGGAATCTGGCATCTGGCT
TTTTATAATGATGGGAAAAATGCAGAGCAGGTGTCTTTTAATACCATTGTTATAGAGTCTGTGGTGGA
ATGTCCCCGAAATTGCCATGGAAATGGAGAATGCGTTTCTGGAACTTGCCATTGTTTTCCAGGATTTC
TGGGTCCGGATTGTTCAAGAGCCGCCTGTCCAGTGTTATGTAGTGGCAACGGGCAGTACTCCAAGGGC
CGCTGCCTGTGTTTCAGCGGCTGGAAGGGCACCGAGTGTGATGTGCCGACTACCCAGTGTATTGACCC
ACAGTGTGGGGGTCGTGGGATTTGTATCATGGGCTCCTGTGCTTGCAGCTCAGGATACAAAGGAGAAA
GTTGTGAAGAAGCTGACTGTATAGACCCTGGGTGTTCTAATCATGGTGTGTGTATCCACGGGGAATGT
CACTGCAGTCCAGGATGGGGAGGTAGCAATTGTGAAATACTGAAGACCATGTGTCCAGACCAGTGCTC
CGGCCACGGAACGTATCTTCAAGAAAGTGGCTCCTGCACGTGTGACCCTAACTGGACTGGCCCAGACT
GCTCAAACGAAATATGTTCTGTGGACTGTGGCTCACACGGCGTTTGCATGGGGGGGACGTGTCGCTGT
GAAGAAGGCTGGACGGGCCCAGCCTGTAATCAGAGAGCCTGCCACCCCCGCTGTGCCGAGCACGGGAC
CTGCAAGGATGGCAAGTGTGAATGCAGCCAGGGCTGGAATGGAGAGCACTGCACTATCGCTCACTATT
TGGATAAGATAGTTAAAGACAAGATAGGATATAAAGAGGGTTGTCCTGGTCTGTGCAACAGCAATGGA
AGATGTACCCTGGACCAAAATGGCGGACATTGTGTGTGCCAGCCTGGATGGAGAGGAGCAGGCTGTGA
CGTAGCCATGGAGACTCTTTGCACAGATAGCAAGGACAATGAAGGGGATGGACTCATTGACTGCATGG
ATCCCGATTGCTGCCTACAGAGTTCCTGCCAGAATCAGCCCTATTGTCGGGGACTGCCGGATCCTCAG
GACATCATTAGCCAAAGCCTTCAATCGCCTTCTCAGCAAGCTGCCAAATCCTTTTATGATCGAATCAG
TTTCCTTATAGGATCTGATAGCACCCATGTTATACCTGGAGAAAGTCCTTTCAATAAGAGCCTTGCAT
CTGTCATCAGAGGCCAAGTACTGACTGCTCATGGAACTCCACTTATTGGAGTAAATGTCTCGTTTTTC
CATTACCCAGAATATGGATATACTATTACCCGCCAGGACGGAATGTTTGACTTGGTGGCAAATGGTGG
GGCCTCTCTAACTTTGGTATTTGAACGATCCCCATTCCTCACTCAGTATCATACTGTGTGGATTCCAT
GGAATGTCTTTTATGTGATGGATACCCTAGTCATGGAGAAAGAAGAGAATGACATTCCCAGCTGTGAT
CTGAGTGGATTCGTGAGGCCAAATCCCATCATTGTGTCATCACCTTTATCCACCTTTTTCAGATCTTC
TCCTGAAGACAGTCCCATCATTCCCGAAACACAGGTACTCCACGAGGAAACTACANTTCCAGGAACAG
ATTTGAAACTCTCCTACTTGAGTTCCAGAGCTGCAGGGTATAAGTCAGTTCTCAAGATCACCATGACC
CAGTCTATTATTCCATTTAATTTAATGAAGGTTCATCTTATGGTAGCTGTAGTAGGAAGACTCTTCCA
AAAGTGGTTTCCTGCCTCACCAAACTTGGCCTATACTTTCATATGGGATAAAACAGATGCATATAATC
AGAAAGTCTATGGTCTATCTGAAGCTGTTGTGTCAGTTGGATATGAGTATGAGTCGTGTTTGGACCTG
ACTCTGTGGGAAAAGAGGACTGCCATTCTGCAGGGCTATGAATTGGATGCGTCCAACATGGGTGGCTG
GACATTAGATAAACATCACGTGCTGGATGTACAGAACGGTATACTGTACAAGGGAAACGGGGAAAACC
AGTTCATCTCCCAGCAGCCTCCAGTCGTGAGTAGCATCATGGGCAATGGGCGAAGGCGCAGCATTTCC
TGCCCCAGTTGCAATGGTCAAGCTGATGGTAACAAGTTACTGGCCCCAGTGGCGCTAGCTTGTGGGAT
CGATGGCAGTCTGTACGTAGGCGKTTTCAACTACGTGCGGCGGATATTCCCTTCTGGAAATGTAACAA
GTGTCTTAGAACTAAGAAATAAAGATTTTAGACATAGCAGCAACCCAGCTCATAGATACTACCTTGCA
ACGGATCCAGTCACGGGAGATCTGTACGTTTCTGACACAAACACCCGCAGAATTTATCGCCCAAAGTC
ACTTACGGGGGCAAAAGACTTGACTAAAAATGCAGAAGTCGTCGCAGGGACAGGGGAGCAATGCCTTC
CGTTTGACGAGGCGAGATGTGGGGATGGAGGGAAGGCCGTGGAAGCCACACTCATGAGTCCCAAAGGA
ATGGCAGTTGATAAGAATGGATTAATCTACTTTGTTGATGGAACCATGATTAGGAAAGTTGACCAAAA
TGGAATCATATCAACTCTTCTGGGCTCTAACGATTTGACTTCAGCCAGACCTTTAACTTGTGACACCA
GCATGCACATCAGCCAGGTACGTCTGGAATGGCCCACTGACCTAGCCATTAACCCTATGGATAACTCC
ATTTATGTCCTGGATAATAATGTAGTfflACAGATCACTGAAAATCGTCAAGTTCGCATTGCTGCTGG
ACGGCCCATGCACTGTCAGGTTCCCGGAGTGGAATATCCTGTGGGGAAGCACGCGGTGCAGACAACAC
TGGAATCAGCCACTGCCATTGCTGTGTCCTACAGTGGGGTCCTGTACATTACTGAAACTGATGAGAAG
AAAATfAACCGGATAAGGCAGGTCACAACAGATGGAGAAATCTCCTTAGTGGCCGGAATACCTTCAGA
GTGTGACTGCAAAAATGATGCCAACTGTGACTGTfACCAGAGTGGAGATGGCTACGCCAAGGATGCCA
AACTCAGTGCCCCATCCTCCCTGGCTGCTTCTCCAGATGGTACACTGTATATTGCAGATCTAGGGAAT
ATCCGGATCCGGGCTGTGTCAAAGAATAAGCCTTTACTTAACTCTATGAACTTCTATGAAGTTGCGTC
TCCAACTGATCAAGAACTCTACATCTTTGACATCAATGGTACTCACCAATATACTGTAAGTTTAGTCA
CTGGTGATTACCTTTACAATTTTAGCTACAGCAATGACAATGATATTACTGCTGTGACAGACAGCAAT
GGCAACACCCTTAGAATTAGACGGGACCCAAATCGCATGCCAGTTCGAGTGGTGTCTCCTGATAACCA
AGTGATATGGTTGACAATAGGAACAAATGGATGTTTGAAAGGCATGACTGCTCAAGGACTGGAATTAG
TTTTGTTTACTTACCATGGCAATAGTGGCCTTTTAGCCACTAAAAGTGATGAAACTGGATGGACAACG
TTTTTTGACTATGACAGTGAAGGTCGTCTGACAAATGTTACGTTTCCAACTGGAGTGGTCACAAACCT
GCATGGGGACATGGACAAGGCTATCACAGTGGACATTGAGTCATCTAGCCGAGAAGAAGATGTCAGCA
TCACTTCAAATCTGTCCTCGATCGATTCTTTCTACACCATGGTTCAAGATCAGTTAAGAAACAGCTAC
CAGATTGGTTATGACGGCTCCCTCAGAATTATCTACGCCAGTGGCCTGGACTCACACTACCAAACAGA
GCCGCACGTTCTGGCTGGCACCGCTAATCCGACGTTTGCCAAAAGAAACATGACTTTGCCTGGCGAGA
ACGGTCAAAACTTGGTGGAATGGAGATTCCGAAAAGAGCAAGCCCAAGGGAAAGTCAATGTCTTTGGC
CGCAAGCTCAGGGTTAATGGCAGAAACCTCCTTTCAGTfGACTTTGATCGAACAACAAAGACAGAAAA
GATCTATGACGACCACCGTAAATTTCTACTGAGGATCGCCTACGACACGTCTGGGCACCCGACTCTCT
GGCTGCCAAGCAGCAAGCTGATGGCCGTCAATGTCACCTATTCATCCACAGGTCAAATTGCCAGCATC
CAGCGAGGCACCACTAGCGAGAAAGTAGATTATGACGGACAGGGGAGGATCGTGTCTCGGGTCTTTGC
TGATGGTAAAACATGGAGTTACACATATTTAGAAAAGTCCATGGTTCTTCTGCTTCATAGCCAGCGGC
AGTACATCTTCGAATACGATATGTGGGACCGCCTGTCTGCCATCACCATGCCCAGTGTGGCTCGCCAC
ACCATGCAGACCATCCGATCCATTGGCTACTACCGCAACATATACAACCCCCCGGAAAGCAACGCCTC
CATCATCACGGACTACAACGAGGAAGGGCTGCTTCTACAAACAGCTTTCTTGGGTACAAGTCGGAGGG
TCTTATTCAAATACAGAAGGCAGACTAGGCTCTCAGAAATTTTATATGATAGCACAAGAGTCAGTTTT
ACCTATGATGAAACAGCAGGAGTCCTAAAGACAGTAAACCTCCAGAGTGATGGTTTTATTTGCACCAT
TAGATACAGGCAAATTGGTCCCCTGATTGACAGGCAGATTTTCCGCTTTAGTGAAGATGGGATGGTAA
ATGCAAGATTTGACTATAGCTATGACAACAGCTTTCGAGTGACCAGCATGCAGGGTGTGATCAATGAA
ACGCCACTGCCTATTGATCTGTATCAGTTTGATGACATTTCTGGCAAAGTTGAGCAGTTTGGAAAGTT
TGGAGTTATATATTATGATATTAACCAGATCATETCTACAGCTGTAATGACCTATACGAAGCACTTTG
ATGCTCATGGCCGTATCAAGGAGATTCAATATGAGATATTCAGGTCGCTCATGTACTGGATTACAATT
CAGTATGATAACATGGGTCGGGTAACCAAGAGAGAGATTAAAATAGGGCCCTTTGCCAACACCACCAA
ATATATGCTTATGAATATGATGTTGATGGACAGCTCCAAACACAGTTTACCTCAATGGATAATGTGGC
GGTACAACTACGATCTGAATGGAAACCTCCATTTACTGAACCCAAGTAACAGTGCGCGTCTGACACCC
CTTCGCTATGACCTGCGAGACAGAATCACTCGACTGGGTGATGTTCAATATCGGTTGGATGAAGATGG
TTTCCTACGTCAAAGGGGCACGGAAATCTTTGAATATAGCTCCAAGGGGCTTCTAACTCGAGTTTACA
GTAAAGGCAGTGGCTGGACAGTGATCTACCGTTATGACGGCCTGGGAAGGCGTGTTTCTAGCAAAACC
AGTCTAGGACAGCACCTGCAGTTTTTTTATGCTGACTTAACTTATCCCACTAGGATTACTCATGTCTA
CAACCATTCGAGTTCAGAAATTACCTCCCTGTATTATGATCTCCAAGGACATCTTTTTGCCATGGAAA
TCAGCAGTGGGGATGAATTCTATATTGCATCGGATAACACAGGGACACCACTGGCTGTGTTCAGTAGC
AATGGGCTTATGCTGAAACAGATTCAGTACACTGCATATGGGGAAATCTATTTTGACTCTAATATTGA
CTTTCAACTGGTAATTGGATTTCATGGTGGCcTGTATGACCCACTCACCAAATTAATCCACTTTGGAG
AAAGAGATTATGACATTTTGGCAGGACGGTGGACAACACCTGACATAGAAATCTGGAAAAGAATTGGG
AAGGACCCAGCTCCTTTTAACTTGTACATGTTTAGGAATAACAACCCTGCAAGCAAAATCCATGACGT
GAAAGATTACATCACAGATGTTAACAGCTGGCTGGTGACATTTGGTTTCCATCTGCACAATGCTATTC
CTGGATTCCCTGTTCCCAAATTTGATTTAACAGAACCTTCTfACGAACTfGTGAAGAGTCAGCAGTGG
GATGATATACCGCCCATCTTCGGAGTCCAGCAGCAAGTGGCGCGGCAGGCCAAGGCCTTCCTGTCGCT
GGGGAAGATGGCCGAGGTGCAGGTGAGCCGGCGCCGGGCCGGCGGCGCGCAGTCCTGGCTGTGGTTCG
CCACGGTCAAGTCGCTGATCGGCAAGGGCGTCATGCTGGCCGTCAGCCAGGGCCGCGTGCAGACCAAC
GTGCTCAACATCGCCAACGAGGACTGCATCAAGGTGGCGGCCGTGCTCAACAACGCCTTCTACCTGGA
GAACCTGCACTTCACCATCGAGGGCAAGGACACGCACTACTTCATCAAGACCACCACGCCCGAGAGCG
ACCTGGGCACGCTGCGGTTGACCAGCGGCCGCAAGGCGCTGGAGAACGGCATCAACGTGACGGTGTCG
CAGTCCACCACGGTGGTGAACGGCAGGACGCGCAGGTTCGCGGACGTGGAGATGCAGTTCGGCGCGCT
GGCGCTGCACGTGCGCTACGGCATGACCCTGGACGAGGAGAAGGCGCGCATCCTGGAGCAGGCGCGGC
AGCGCGCGCTCGCCCGGGCCTGGGCGCGCGAGCAGCAGCGCGTGCGCGACGGCGAGGAGGGCGCGCGC
CTCTGGACGGAGGGCGAGAAGCGGCAGCTGCTGAGCGCCGGCAAGGTGCAGGGCTACGACGGGTACTA
CGTACTCTCGGTGGAGCAGTACCCCGAGCTGGCCGACAGCGCCAACAACATCCAGTTCCTGCGGCAGA
GCGAGATCGGCAGGAGGTAACGCCCGGGCCGCGCCCGCCGAGCCGCTCACGCCCTGCCCACATTGTCC
TGTGGCACAACCCGAGTGGGACTCTCCAACGCCCAAGAGCCTTCCTCCCGGGGGAATGAGACTGCTGT
TACGACCCACACCCACACCGCGAAAACAAGGACCGCTTTTTTCCGAATGACCTTAAAGGTGATCGGCT
TTAACGAATATGTTTACATATGCATAGCGCTGCACTCAGTCGGACTGAACGTAGCCAGAGGAAAAAAA
AATCATCAAGGACAAAGGCCTCGACCTGTTGCGCTGGGCCGTCTGTTCCTTCTAGGCACTGTATTTAA
CTAACTTTA

| | | | |
|---|---|---|---|
| CG55069-02 | SEQ ID NO:10 | 2721 aa | MW at 303489.1kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENEQPASNQGQSTLQPLPPSHKQHSAQHHPSITSLN
RNSLTNRRNQSPAPPAALPAELQTTPESVQLQDSWVLGSNVPLESRHFLFKTGTGTTPLFSTATPGYT
MASGSVYSPPTRPLPRNTLSRSAFKFKKSSKYCSWKCTALCAVGVSVLLAILLSYFIAMHLFGLNWQL
QQTENDTFENGKVNSDTMPTNTVSLPSGDNGKLGGFTQENNTIDSGELDIGRRAIQELPPGIFWRSQL
FIDQPQFLKFNISLQKDALIGVYGRKGLPPSHTQYDFVELLDGSRLIAREQRSLLETERAGRQARSVS
LHEAGFIQYLDSGIWHLAFYNDGKNAEQVSFNTIVLESVVECPRNCHGNGECVSGTCHCFPGFLGPDC
SRAACPVLCSGNGQYSKGRCLCFSGWKGTECDVPTTQCIDPQCGGRGICIMGSCACSSGYKGESCEEA
DCIDPGCSNHGVCIHGECHCSPGWGGSNCEILKTMCPDQCSGHGTYLQESGSCTCDPNWTGPDCSNEI
CSVDCGSHGVCMGGTCRCEEGWTGPACNQRACHPRCAEHGTCKDGKCECSQGWNGEHCTIAHYLDKIV
KDKIGYKEGCPGLCNSNGRCTLDQNGGHCVCQPGWRGAGCDVAMETLCTDSKDNEGDGLIDCMDPDCC
QVLTADGTPLIGVNVSFFHYPEYGYTITRQDGMFDLVANGGASLTLVFERSPFLTQYHTVWLPWNVFY
VMDTLVMEKEENDLPSCDLSGFVRPNPLIVSSPLSTFFRSSPEDSPIIPETQVLHEETTLPGTDLKLS
YLSSRAAGYKSVLKITMTQSIIPFNLMKVHLMVAVVGRLFQKWFPASPNLAYTFIWDKTDAYNQKVYG
LSEAVVSVGYEYESCLDLTLWEKRTAILQGYELDASNMGGWTLDKHHVLDVQNGILYKGNGENQFISQ
QPPVVSSIMGNGRRRSISCPSCNGQADGNKLLAPVALACGIDGSLYVGDFNYVRRIFPSGNVTSVLEL
RNKDFRHSSNPAHRYYLATDPVTGDLYVSDTNTRRIYRPKSLTGAKDLTKNAEVVAGTGEQCLPFDEA
RCGDGGKAVEATLMSPKGMAVDKNGLIYFVDGTMIRKVDQNGIISTLLGSNDLTSARPLTCDTSMHIS
QVRLEWPTDLAINPMDNSIYVLDNNVVLQITENRQVRIAAGRPMHCQVPGVEYPVGKHAVQTTLESAT
AIAVSYSGVLYITETDEKKINRIRQVTTDGEISLVAGIPSECDCKNDANCDCYQSGDGYAKDAKLSAP
SSLAASPDGTLYIADLGNIRIRAVSKNKPLLNSMNFYEVASPTDQELYJFDINGTHQYTVSLVTGDYL
YNFSYSNDNDITAVTDSNGNTLRIRRDPNRMPVRVVSPDNQVIWLTIGTNGCLKGMTAQGLELVLFTY
HGNSGLLATKSDETGWTTFFDYDSEGRLTNVTFPTGVVTNLHGDMDKAITVDIESSSREEDVSITSNL
SSIDSFYTMVQDQLRNSYQIGYDGSLRIIYASGLDSHYQTEPHVLAGTANPTVAKRNMTLPGENGQNL
VEWRFRKEQAQGKVNVFGRKLRVNGRNLLSVDFDRTIKTEKIYDDHRKFLLRIAYDTSGHPTLWLPSS
KLMAVNVTYSSTGQIASIQRGTTSEKVDYDGQGRIVSRVFADGKTWSYTYLEKSMVLLLHSQRQYIFE
YDMWDRLSAITMPSVARHTMQTIRSIGYYRNIYNPPESNASIITDYNEEGLLLQTAFLGTSRRVLFKY
RRQTRLSEILYDSTRVSFTYDETAGVLKTVNLQSTGFICTIRYRQIGPLIDRQIFRFSEDGMVNARFD
IKEIQYEIFRSLMYWITIQYDNMGRVTKREIKIGPFANITKYAYEYDVDGQLQTVYLNEKIMWRYNYD
LNGNLHLLNPSNSARLTPLRYDLRDRITRLGDVQYRLDEDGFLRQRGTEIFEYSSKGLLTRVYSKGSG
WTVIYRYDGLGRRVSSKTSLGQHLQFFYADLTYPTRITHVYNHSSSEITSLYYDLQGHLFAMEISSGD
EFYIASDNTGTPLAVFSSNGLMLKQIQYTAYGEIYFDSNIDFQLVIGFHGGLYDPLTKLIHFGERDYD
ILAGRWTTPDIEIWKRIGKDPAPFNLYMFRNNNPASKIHDVKDYITDVNSWLVTFGFHLHNAJPGFPV
PKFDLTEPSYELVKSQQWDDIPPIFGVQQQVARQAKAFLSLGKMAEVQVSRRRAGGAQSWLWFATVKS
LIGKGVMLAVSQGRVQTNVLNIANEDCIKVAAVLNNAFYLENLHFTIEGKDTHYFIKTITPESDLGTL
RLTSGRKALENGINVTVSQSTTVVNGRTRRFADVEMQFGALALHVRYGMTLDEEKARILEQARQRALA
RAWAREQQRVRDGEEGARLWTEGEKRQLLSAGKVQGYDGYYVLSVEQYPELADSANNIQFLRQSEIGR
R

| | | |
|---|---|---|
| CG55069-04 | SEQ ID NO:11 | 1783 bp |
| DNA Sequence | ORF Start: at 7 | ORF Stop: at 778 |

AAGCTTTGTCCCCGAAATTGCCATGGAAATGGAGAATGCGTTTCTGGAACTTGCCATTGTTTTCCAGG
ATTTCTGGGTCCGGATTGTTCAAGAGCCGCCTGTCCAGTGTTATGTAGTGGCAACGGGCAGTACTCCA
AGGGCCGCTGCCTGTGTTTCAGCGGCTGGAAGGGCACCGAGTGTGATGTGCCGACTACCCAGTGTATT
GACCCACAGTGTGGGGGTCGTGGGATTTGTATCATGGGCTCCTGTGCTTGCAACTCAGGATACAAAGG
AGAAAGTTGTGAAGAAGCTGACTGTATAGACCCTGGGTGTTCTAATCATGGTGTGTGTATCCACGGGG
AATGTCACTGCAGTCCAGGATGGGGAGGTAGCAAFTGTGAAATACTGAAGACCATGTGTCCAGACCAG
TGCTCCGGCCACGGAACGTATCTTCAAGAAAGTGGCTCCTGCACGTGTGACCCTAACTGGACTGGCCC
AGACTGCTCAAACGAAATATGTTCTGTGGACTGTGGCTCACACGGCGTTTGCATGGGGGGGACGTGTC
GCTGTGAAGAAGGCTGGACGGGCCCAGCCTGTAATCAGAGAGCCTGCCACCCCCGCTGTGCCGAGCAC
GGGACCTGCAAGGATGGCAAGTGTGAATGCAGCCAGGGCTGGAATGGAGAGCACTGCACTATCGAGGG
TTGTCCTGGTCTGTGCAACAGCAATGGAAGATGTACCCTGGACCAAAATGGCTGGCATTGTGTGTGCC
AGCCTGGATGGAGAGGAGCAGGCTGTGACGTCGAC

| | | | |
|---|---|---|---|
| CG55069-04 | SEQ ID NO:12 | 257 aa | MW at 26866.7kD |
| Protein Sequence | | | |

CPRNCHGNGECVSGTCHCFPGFLGPDCSRAACPVLCSGNGQYSKGRCLCFSGWKGTECDVPTTQCIDP
QCGGRGICIMGSCACNSGYKGESCEEADCIDPGCSNHGVCIHGECHCSPGWGGSNCEILKTMCPDQCS
GHGTYLQESGSCTCDPNWTGPDCSNEICSVDCGSHGVCMGGTCRCEEGWTGPACNQRACHPRCAEHGT
CKDGKCECSQGWNGEHCTIEGCPGLCNSNGRCTLDQNGWHCVCQPGWRGAGCD

| | |
|---|---|
| CG55069-07 SEQ ID NO:13 | 1833bp |
| DNA Sequence ORF Start: at 7 ORF Stop: at 1828 | |

AAGCTTGACCAAAATGGCGGACATTGTGTGTGCCAGCCTGGATGGAGAGGAGCAGGCTGTGACGTAGC
CATGGAGACTCTTTGCACAGATAGCAAGGACAATGAAGGAGATGGACTCATTGACTGCATGGATCCCG
ATTGCTGCCTACAGAGTTCCTGCCAGAATCAGCCCTATTGTCGGGGACTGCCGGATCCTCAGGACATC
ATTAGCCAAAGCCTTCAATCGCCTTCTCAGCAAGCTGCCAAATCCTTTTATGATCGAATCAGTTTCCT
TATAGGATCTGATAGCACCCATGTTATACCTGGAGAAAGTCCTTTCAATAAGAGCCTTGCATCTGTCA
TCAGAGGCCAAGTACTGACTGCTGATGGAACTCCACTTATTGGAGTAAATGTCTCGTTTTTCCATTAC
CCAGAATATGGATATACTATTACCCGCCAGGACGGAATGTTTGACTTGGTGGCAAATGGTGGGGCCTC
TCTAACTTTGGTATETGAACGATCCCCATTCCTCACTCAGTATCATACTGTGTGGATTCCATGGAATG
TCTTTTATGTGATGGATACCCTAGTCATGAAGAAAGAAGAGAATGACATTCCCAGCTGTGATCTGAGT
GGATTCGTGAGGCCAAATCCCATCATTGTGTCATCACCTTTATCCACCTETTTCAGATCTTCTCCTGA
AGACAGTCCCATCATTCCCGAAACACAGGTACTCCACGAGGAAACTACAAYTCCAGGAACAGATTTGA
AACTCTCCTACTTGAGTTCCAGAGCTGCAGGGTATAAGTCAGTTCTCAAGATCACCATGACCCAGTCT
ATTATTCCATTTAATTTAATGAAGGTTCATCTTATGGTAGCTGTAGTAGGAAGACTCTTCCAAAAGTG
GTTTCCTGCCTCACCAAACTTGGCCTATACTTTCATATGGGATAAAACAGATGCATATAATCAGAAAG
TCTATGGTCTATCTGAAGCTGTTGTGTCAGTTGGATATGAGTATGAGTCGTGTTTGGACCTGACTCTG
TGGGAAAAGAGGACTGCCATTCTGCAGGGCTATGAATTGGATGCGTCCAACATGGGTGGCTGGACATT
AGATAAACATCACGTGCTGGATGTACAGAACGGTATACTGTACAAGGGAAACGGGGAAAACCAGTTCA
TCTCCCAGCAGCCTCCAGTCGTGAGTAGCATCATGGGCAATGGGCGAAGGCGCAGCATTTCCTGCCCC
AGTTGCAATGGTCAAGCTGATGGTAACAAGTTACTGGCCCCAGTGGCGCTAGCTTGTGGGATCGATGG
CAGTCTGTACGTAGGCGATTTCAACTATGTGCGGCGGATATTCCCTTCTGGAAATGTAACAAGTGTCT
TAGAACTAAGCAGCAACCCAGCTCATAGATACTACCTTGCAACGGATCCAGTCACGGGAGATCTGTAC
GTTTCTGACACAAACACCCGCAGAATTTATCGCCCAAAGTCACTTACGGGGGCAAAAGACTTGACTAA
AAATGCAGAAGTCGTCGCAGGGACAGGGGAGCAATGCCTTCCGTTTGACGAGGCGAGATGTGGGGATG
GAGGGAAGGCCGTGGAAGCCACACTCATGAGTCCCAAAGGAATGGCAGTTGATAAGAATGGATTAATC
TACTTTGTTGATGGAACCATGATTAGGAAAGTTGACCAAAATGGAATCATATCAACTCTTCTGGGTTC
TAACGATTTGACTTCAGCCAGACCTETAACTTGTGACACCAGCATGCACATCAGCCAGGTACGTCTGG
AATGGCCCACTGACCTAGCCATTAACCCTATGGATAACTCCATTTTATGTCCTGGATAATGTCGAC

| | | | |
|---|---|---|---|
| CG55069-07 | SEQ ID NO:14 | 607 aa | MW at 66606.6kD |
| Protein Sequence | | | |

DQNGGHCVCQPGWRGAGCDVAMETLCTDSKDNEGDGLIDCMDPDCCLQSSCQNQPYCRGLPDPQDIIS
QSLQSPSQQAAKSFYDRISFLIGSDSTHVIIGESPFNKSLASVLRGQVLTADGTPLIGVNVSFFHYPE
YGYTITRQDGMFDLVANGGASLTLVFERSPFLTQYHTVWLPWNVFYVMDTLVMKKEENDTPSCDLSGF
VRPNPIIVSSPLSTFFRSSPEDSPIIPETQVLHEETTIPGTDLKLSYLSSRAAGYKSVLKITMTQSII
PFNLMKVHLMVAVVGRLFQKWFPASPNLAYTFIWDKTDAYNQKVYGLSEAVVSVGYEYESCLDLTLWE
KRTAILQGYELDASNMGGWTLDKHHVLDVQNGILYKGNGENQFISQQPPVVSSLMGNGRRRSISCPSC
NGQADGNKLLAPVALACGIDGSLYVGDFNYVRRIFPSGNVTSVLELSSNPAHRYYLATDPVTGDLYVS
DTNTRRIYRPKSLTGAKDLTKNAEVVAGTGEQCLPFDEARCGDGGKAVEATLMSPKGMAVDKNGLIYF
VDGTMIRKVDQNGIISTLLGSNDLTSARPLTCDTSMHISQVRLEWPTDLAINPMDNSIYVLDN

| | | |
|---|---|---|
| CG55069-15 | SEQ ID NO:15 | 768 bp |
| DNA Sequence | ORF Start: ATG at 65 | ORF Stop: TAA at 707 |

AGACTTGAACCCTGAGCCTAAGTTGTCACCAGCAGGACTGATGTGCACACAGAAGGAATGAAGTATGG
ATGTGAAAGAACGCAGGCCTTACTGCTCCCTGACCAAGAGCAGACGAGAGAAGGAACGGCGCTACACA
GAAAGCTTTTGATCATGATTCCTCGCGGCTGGTTTACGGCAACAGAGTGAAGGATTTGGTTCACAGAG
AAGCAGACGAGTTCACTAGACAAGGACAGAATTTTACCCTAAGGCAGTTAGGAGTTTGTGAACCAGCA
ACTCGAAGAGGACTGGCATTTTGTGCGGAAATGGGGCTCCCTCACAGAGGTTACTCTATCAGTGCAGG
GTCAGATGCTGATACTGAAAATGAAGCAGTGATGTCCCCAGAGCATGCCATGAGACTTTGGGGCAGGG
GGGTCAAATCAGGCCGCAGCTCCTGCCTGTCAAGTCGGTCCAACTCAGCCCTCACCCTGACAGATACG
GAGCACGAAAACAAGTCCGACAGTGAGAATGGAGGGTCAAGCAGTTGGTTCGGTTTTCATTGGAATTT
TTATGTGGGTAAAGCTTCCTGTTTGCTGCGCTTGCCTAGGATTTTCTTATCCCACAACTACAATGTGA
ACAAAGAGATGAGAGAGAAATTATGCTAATGCATTTTGGTGGATCAATGCTAATGCATTTTGGTGGAT
CAATGCTAATGCATTTTGGT

| | | | |
|---|---|---|---|
| NOV1o, CG55069-15 | SEQ ID NO:16 | 214 aa | MW at 24376.8kD |
| Protein Sequence | | | |

MDVKERRPYCSLTKSRREKERRYTNSSADNEECRVPTQKSYSSSETLKAFDHDSSRLLYGNRVKDLVH
READEFTRQGQNFTLRQLGVCEPATRRGLAFCAEMGLPHRGYSISAGSDADTENEAVMSPEHAMRLWG
RGVKSGRSSCLSSRSNSALTLTDTEHENKSDSENGGSSSWFGFHWNFYVGKASCLLRLPRIFLSHNYN
VNKEMREKLC

| | | |
|---|---|---|
| CG55069-18 | SEQ ID NO:17 | 908 bp |
| DNA Sequence | | |

GACGCGGCCCAGCCGGCCAGGCGCGCGCGCCGTACGAAGCTTTGTCCCCGAAATTGCCATG
GAAATGGAGAATGCGTTTCTGGAACTTGCCATTGTTTTCCAGGATTTCTGGGTCCGGATT
GTTCAAGAGCCGCCTGTCCAGTGTTATGTAGTGGCAACGGGCAGTACTCCAAGGGCCGC
TGCCTGTGTFTCAGCGGCTGGAAGGGCACCGAGTGTGATGTGCCGACTACCCAGTGTAT
TGACCCACAGTGTGGGGGTCGTGGGATTTGTATCATGGGCTCCTGTGCTTGCAACTCAG
GATACAAAGGAGAAAGTTGTGAAGAAGCTGACTGTATAGACCCTGGGTGTTCTAATCAT
GGTGTGTGTATCCACGGGGAATGTCACTGCAGTCCAGGATGGGGAGGTAGCAATTGTGA
AATACTGAAGACCATGTGTCCAGACCAGTGCTCCGGCCACGGAACGTATCTTCAAGAAA
GTGGCTCCTGCACGTGTGACCCTAACTGGACTGGCCCAGACTGCTCAAACGAAATATGT
TCTGTGGACTGTGGCTCACACGGCGTTTGCATGGGGGGGACGTGTCGCTGTGAAGAAG
GCTGGACGGGCCCAGCCTGTAATCAGAGAGCCTGCCACCCCCGCTGTGCCGAGCACGG
GACCTGCAAGGATGGCAAGTGTGAATGCAGCCAGGGCTGGAATGGAGAGCACTGCACT
ATCGAGGGTTGTCCTGGTCTGTGCAACAGCAATGGAAGATGTACCCTGGACCAAAATGG
CTGGCATTGTGTGTGCCAGCCTGGATGGAGAGGAGCAGGCTGTGACCTCGAGGGTAAGC
CTATCCCTAACCCTCTCCTCGGTCTCGATTCTACGCGTACCGGTCATCATCACCATCACCATTGA

| | | |
|---|---|---|
| CG55069-18 | SEQ ID NO:18 | 296 aa |
| Protein Sequence | | |

DAAQPARRARRTKLCPRNCHGNGECVSGTCHCFPGFLGPDCSRAACPVLCSGNGQYSKGRC
LCFSGWKGTECDVPTTQCIDPQCGGRGICIMGSCACNSGYKGESCEEADCIDPGCSNHGVCI
HGECHCSPGWGGSNCEILKTMCPDQCSGHGTYLQESGSCTCDPNWTGPDCSNEICSVDCGS
HGVCMGGTCRCEEGWrGPACNQRACHPRCAEHGTCKDGKCECSQGWNGEHCTLEGCPGL
CNSNGRCTLDQNGWHCVCQPGWRGAGCDLEGKPIPNPLLGLDSTRTGHHHHHH

| | | |
|---|---|---|
| CG55069-19 | SEQ ID NO:19 | 2589 aa |
| DNA Sequence | | |

GACGCGGCCCAGCCGGCCAGGCGCGCGCGCCGTACGAAGCTTTCGCGAAACTGGCAGCTACAG
CAGACTGAAAATGACACATTTGAGAATGGAAAAGTGAATTCTG
ATACCATGCCAACAAACACTGTGTCATTACCTTCTGGAGACAATGGAAAATTAGGTGGATTTACGCAA
GAAAATAACACCATAGATTCCGGAGAACTTGATATTGOCCGAAGAGCAATTCAAGAGATTCCTCCCGG
GATCTTCTGGAGATCACAGCTCTTCATTGATCAGCCACAGTTTCTTAAATTCAATATCTCTCTTCAGA
AGGATGCATTGATTGGAGTATATGGCCGGAAAGGCTTACCGCCTTCCCATACTCAGTATGACTTCGTG
GAGCTCCTGGATGGCAGCAGGCTGATTGCCAGAGAGCAGCGGAGCCTGCTTGAGACGGAGAGAGCCGG
GCGGCAGOCGAGATCCGTCAGCCTTCATGAGGCCGGCTTTATCCAGTACTTGGATTCTGGAATCTGGC
ATCTGGCTTTTTATAATGATGGGAAAAATGCAGAGCAGGTGTCTTTTAATACCATTGTTATAGAGTCT
GTGGTGGAATGTCCCCGAAATTGCCATGGAAATGGAGAATGCGTTTCTGGAACTTGCCATTGTTTTCC
AGGATTTCTGGGTCCGGATTGTTCAAGAGCCGCCTGTCCAGTGTTATGTAGTGGCAACGGGCAGTACT
CCAAGGGCCGCTGCCTGTGTTTCAGCGGCTGGAAGOOCACCGAGTGTGATGTGCCGACTACCCAGTGT
ATTGACCCACAGTGTGGGGGTCGTGGGATTTGTATCATGGGCTCTTGTGCTTGCAACTCAGGATACAA
AGGAGAAAGTTGTGAAGAAGCTGACTGTATAGACCCTGGGTGTTCTAATCATGGTGTGTGTATCCACG
GGGAATGTCACTGCAGTCCAGGATGGGGAGGTAGCAATTGTGAAATACTGAAGACCATGTGTCCAGAC
CAGTGCTCCGGCCACGGAACGTATCTTCAAGAAAGTGGCTCCTGCACGTGTGACCCTAACTGGACTGG
CCCAGACTGCTCAAACGAAATATGTTCTGTGGACTGTGGCTCACACGGCGTTTGCATGGGGGGGACGT
GTCGCTGTGAAGAAGGCTGGACGGGCCCAGCCTGTAATCAGAGAGCCTGCCACCCCCGCTGTGCCGAG
CACGGGACCTGCAAGGATGGCAAGTGTGAATGCAGCCAGGGCTGGAATGGAGAGCACTGCACTATCGC
TCACTATTTGGATAAGATAGTTAAAGAGGGTTGTCCTGGTCTGTGCAACAGCAATGGAAGATGTACCC
TGGACCAAAATGGCTGOCATTGTGTGTGCCAGCCTGGATGGAGAGGAGCAGGCTGTGACGTAGCCATG
GAGACTCTTTGCACAGATAGTAAGGACAATGAAGGAGATGGACTCATTGACTGCATGGATCCCGAYTG
CTGCCTACAGAGTTCCTGCCAGAATCAGCCCTATTGTCGGOGACTGCCGGATCCTCAGGACATCATTA
GCCAAAGCCTTCAATCGCCTTCTCAGCAAGCTGCCAAATCCTTTTATGATCGAATCAGTTTCCTTATA
GGATCTGATAGCACCCATGTTATACCTGGAGAAAGTCCTTTCAATAAGAGCCTTGCATCTGTCATCAG
AGGCCAAGTACTGACTGCTGATGGAACTCCACTTATTGGAGTAAATGTCTCGTTTTTCCATTACCCAG
AATATGGATATACTATTACCCGCCAGGACGGAATGTTTGACTTGGTGGCAAATGGTGGGGCCTCTCTA
ACTTTGGTATTTGAACGATCCCCATTCCTCACTCAGTATCATACTGTGTGGATTCCATGGAATGTCTT
TTATGTGATGGATACCCTAGTCATGAAGAAAGAAGAGAATGACATTCCCAGCTGTGATCTGAGTGGAT
TCGTGAGGCCAAATCCCATCATTGTGTCATCACCTTTATCCACCTTTTTCAGATCTTCTCCTGAAGAC
AGTCCCATCATTCCCGAAACACAGGTACTCCACGAGGAAACTACAATTCCAGGAACAGATTTGAAACT
CTCCTACTTGAGTTCCAGAGCTGCAGGGTATAAGTCAGTTCTCAAGATCACCATGACCCAGTCTATTA
TTCCATTTAATTTAATGAAGGTTCATCTTATGGTAGCTGTAGTAGGAAGACTCTTCCAAAAGTGGTTT
CCTGCCTCACCAAACTTGGCCTATACTTTCATATGGGATAAAACAGATGCATATAATCAGAAAGTCTA
TGGTCTATCTGAAGCTGTTGTGTCAGTTGGATATGAGTATGAGTCGTGTTTGGACCTGACTCTGTGOG
AAAAGAGGACTGCCATTCTGCAGGGCTATGAATTGGATGCGTCCAACATGGGTGGCTGGACATTAGAT
AAACATCACGTGCTGGATGTACAGAACGGTATACTGTACAAGOGAAACGGGGAAAACCAGTTCATCTC
CCAGCAGCCTCCAGTCGTGAGTAGCCTCGAGGGTAAGCCTATCCCTAACCCTCTCCTCGGTCTCGATTCT
ACGCGTACCGGTCATCATCACCATCACCATTGA

| | | |
|---|---|---|
| CG55069-19 | SEQ ID NO:20 | 862 aa |
| Protein Sequence | | |

DAAQPARRARRTKLSRNWQLQQTENDTFENGKVNSDTMPTNTVSLPSGDNGKLGGFTQENNTIDS
GELDIGRRAIQEIPPGIFWRSQLFIDQPQFLKFNISLQKDALIGVYGRKGLPPSHTQYDFVELLDGSRL
IAREQRSLLETERAGRQARSVSLHEAGFIQYLDSGIWHLAFYNDGKNAEQVSFNTIVLESVVECPRNC
HGNGECVSGTCHCFPGFLGPDCSRAACPVLCSGNGQYSKGRCLCFSGWKGTECDVPTTQCIDPQCG
GRGICIMGSCACNSGYKGESCEEADCIDPGCSNHIGVCIHGECHCSPGWGGSNCEILKTMCPDQCSGH
GTYLQESGSCTCDPNWTGPDCSNEICSVDCGSHGVCMGGTCRCEEGWTGPACNQRACHPRCAEHGT
CKDGKCECSQGWNGEHCTIAHYLDKIVKEGCPGLCNSNGRCTLDQNGWHCVCQPGWRGAGCDVA
METLCTDSKDNEGDGLIDCMDPDCCLQSSCQNQPYCRGLPDPQDIISQSLQSPSQQAAKSFYDRISFLIG
SDSTHVLPGESPFNKSLASVIRGQVLTADGTPLIGVNVSFFHYPEYGYTITRQDGMFDLVANGGASLTL
VFERSPFLTQYHTVWIPWNVFYVMDTLVMKKEENDLPSCDLSGFVRPNPIIVSSPLSTFFRSSPEDSPIIPE
TQVLHEE1TIPGTDLKLSYLSSRAAGYKSVLKITMTQSIIPFNLMKVHLMVAVVGRLFQKWFPASPNLA
YTFIWDKTDAYNQKVYGLSEAVVSVGYEYESCLDLTLWEKRTAILQGYELDASNMGGWTLDKHHVLD
VQNGILYKGNGENQFISQQPPVVSSLEGKPLPNPLLGLDSTRTGHHHHHH

### Example 2

### Domain Analysis of CG55069-17

TABLE 3
Domain analysis of CG55069-17 (Pfam)
Parsed for domains:

| Model | Domain | seq-f | seq-t | | score | E-value |
|---|---|---|---|---|---|---|
| Ten_N | 1/1 | 10 | 308 | . . . | 848.6 | 2.1e−251 |
| EGF | 1/8 | 518 | 544 | . . . | 17.3 | 0.36 |
| EGF_2 | 1/8 | 518 | 544 | . . . | 27.7 | 0.00026 |
| EGF | 2/8 | 549 | 575 | . . . | −1.4 | 26 |
| EGF_2 | 2/8 | 549 | 575 | . . . | 17.5 | 0.24 |
| EGF | 3/8 | 582 | 609 | . . . | 17.6 | 0.3 |
| EGF_2 | 3/8 | 582 | 609 | . . . | 25.6 | 0.0011 |
| EGF | 4/8 | 614 | 641 | . . . | 23.3 | 0.0058 |
| EGF_2 | 4/8 | 614 | 641 | . . . | 22.6 | 0.0096 |
| EGF_2 | 5/8 | 648 | 676 | . . . | 19.1 | 0.1 |
| EGF | 5/8 | 648 | 676 | . . . | 11.3 | 1.5 |
| EGF_alliinase | 1/1 | 634 | 685 | . . . | −14.5 | 4.5 |
| EGF | 6/8 | 681 | 707 | . . . | 16.0 | 0.51 |
| EGF_2 | 6/8 | 681 | 707 | . . . | 22.6 | 0.0094 |
| Keratin_B2 | 1/1 | 571 | 730 | . . . | −85.6 | 4.8 |
| EGF_2 | 7/8 | 712 | 738 | . . . | 27.0 | 0.00043 |
| EGF | 7/8 | 712 | 738 | . . . | 24.1 | 0.0033 |
| DSL | 1/1 | 677 | 738 | . . . | −20.8 | 8.4 |
| EGF | 8/8 | 752 | 782 | . . . | 19.6 | 0.074 |
| EGF_2 | 8/8 | 752 | 782 | . . . | 19.8 | 0.067 |
| NHL | 1/5 | 1181 | 1209 | . . . | 0.8 | 79 |
| NHL | 2/5 | 1299 | 1324 | . . . | 5.4 | 20 |
| NHL | 3/5 | 1358 | 1384 | . . . | 14.6 | 1.4 |
| NHL | 4/5 | 1418 | 1445 | . . . | 0.9 | 75 |
| NHL | 5/5 | 1487 | 1514 | . . . | 20.6 | 0.037 |
| RHS_repeat | 1/6 | 1563 | 1600 | . . . | 11.4 | 10 |
| RHS_repeat | 2/6 | 1626 | 1664 | . . . | 12.7 | 6.8 |
| DPPIV_N | 1/1 | 1227 | 1801 | . . . | −205.8 | 5.8 |
| RHS_repeat | 3/6 | 1839 | 1875 | . . . | 10.9 | 12 |
| RHS_repeat | 4/6 | 1944 | 1982 | . . . | 5.0 | 68 |
| RHS_repeat | 5/6 | 2169 | 2207 | . . . | 12.9 | 6.4 |
| RHS_repeat | 6/6 | 2223 | 2261 | . . . | 20.2 | 0.049 |

### Example 3

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s:18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C. greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably. Normalized sscDNA was analyzed by RTQ-PCR using 1× TaqMan® Universal Master mix (Applied Biosystems; catalog No. 4324020), following the manufacturers instructions. PCR amplification and analysis were done as described above.

Panel 1.3D

Panels 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

Panels 2D

Panels 2D included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

Panels 4D

Panels 4D included 2 control wells and 94 test samples of RNA (Panel 4R) or cDNA (Panels 4D and 4.1D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary cell types were activated by incubating with various cytokines (IL-1 beta ˜1-5 ng/ml, TNF alpha ˜5-10 ng/ml, IFN gamma ˜20-50 ng/ml, IL-4 ˜5-10 ng/ml, IL-9 ˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with ˜5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of ˜2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+ lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+ RA and CD45+ RO CD4+ lymphocytes were isolated by depleting mononuclear cells of CD8+, CD56+, CD14+ and CD19+ cells using CD8, CD56, CD14 and CD19 Miltenyi beads and positive selection. CD45RO Miltenyi beads were then used to separate the CD45+RO CD4+ lymphocytes from CD45+RA CD4+ lymphocytes. CD45+RA CD4+, CD45+RO CD4+ and CD8+ lymphocytes were cultured in culture media at 10⁶ cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or ˜10 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 45 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wisc.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

Expression of gene CG55069-17 was assessed using the primer-probe sets Ag1479, Ag2674, Ag2820, Ag757 and Ag939, described in Tables AA, AB, AC, AD and AE. Results of the RTQ-PCR runs are shown in Tables AF, AG and AH.

TABLE 4
Probe Name Ag1479

| | | | | |
|---|---|---|---|---|
| Primers | Sequences | Length | Start Position | SEQ ID No |

| | | | | |
|---|---|---|---|---|
| Forward | 5′-cacggaacgtatcttcaagaaa-3′ | 22 | 6309 | 21 |
| Probe | TET-5′-ctgcacgtgtgaccctaactggactg-3′-TAMRA | 26 | 6276 | 22 |
| Reverse | 5′-gccacagtccacagaacatatt-3′ | 22 | 6235 | 23 |

TABLE 5
Probe Name Ag2674

| | | | | |
|---|---|---|---|---|
| Primers | Sequences | Length | Start Position | SEQ ID No |

| | | | | |
|---|---|---|---|---|
| Forward | 5′-acctactcggccactacctaga-3′ | 22 7429 | 24 | |
| Probe | TET-5′-caccctatcaagaagtgcttttaaattca-3′-TAMRA | 29 | 7398 | 25 |
| Reverse | 5′-cagtgcatttccagctacagta-3′ | 22 | 7362 | 26 |

TABLE 6
Probe Name Ag2820

| | | | | |
|---|---|---|---|---|
| Primers | Sequences | Length | Start Position | SEQ ID No |

| | | | | |
|---|---|---|---|---|
| Forward | 5′-cagagaagcagacgagttcact-3′ | 22 | 8068 | 27 |
| Probe | | | | |
| TET-5′-caaggacagaattttaccctaaggca-3′-TAMRA | 26 | 8039 | 28 | |
| Reverse | 5′-gttgctggttcacaaactccta-3′ | 22 | 8015 | 29 |

TABLE 7
Probe Name Ag757

| | | | | |
|---|---|---|---|---|
| Primers | Sequences | Length | Start Position | SEQ ID No |

| | | | | |
|---|---|---|---|---|
| Forward | 5′-cacctactcggccactacct-3′ | 20 | 7432 | 30 |
| Probe | TET-5′-caccctatcaagaagtgcttttaaattca-3′-TAMRA | 29 | 7398 | 31 |
| Reverse | 5′-cacacagtgcagtgcatttc-3′ | 20 | 7353 | 32 |

TABLE 8
Probe Name Ag939

| | | | | |
|---|---|---|---|---|
| Primers | Sequences | Length | Start Position | SEQ ID No |

| | | | | |
|---|---|---|---|---|
| Forward | 5′-gcagctacagcagactgaaaat-3′ | 22 | 7258 | 33 |
| Probe | TET-5′-tctgataccatgccaacaaacactgtg-3′-TAMRA | 27 | 7204 | 34 |
| Reverse | 5′-ccattgtctccagaaggtaatg-3′ | 22 | 7181 | 35 |

TABLE 9
Panel 1.3D
Column A - Rel. Exp.(%) Ag1479, Run 165520101
Column B - Rel. Exp.(%) Ag2674, Run 162554642
Column C - Rel. Exp.(%) Ag2820, Run 165527000
Column D - Rel. Exp.(%) Ag2820, Run 165544916

| Tissue Name | A | B | C | D |
|---|---|---|---|---|
| Liver adenocarcinoma | 16.0 | 15.9 | 17.2 | 8.2 |
| Pancreas | 0.5 | 0.1 | 0.0 | 0.1 |
| Pancreatic ca. CAPAN 2 | 16.2 | 4.9 | 10.4 | 6.3 |
| Adrenal gland | 4.1 | 0.8 | 4.9 | 2.7 |
| Thyroid | 2.0 | 0.8 | 0.6 | 0.2 |
| Salivary gland | 0.2 | 0.1 | 0.0 | 0.1 |
| Pituitary gland | 3.5 | 0.6 | 0.8 | 0.1 |
| Brain (fetal) | 8.7 | 0.6 | 2.3 | 1.1 |
| Brain (whole) | 10.4 | 2.0 | 1.7 | 2.1 |
| Brain (amygdala) | 12.8 | 3.0 | 2.0 | 2.0 |
| Brain (cerebellum) | 10.0 | 1.8 | 0.3 | 0.3 |
| Brain (hippocampus) | 17.7 | 5.0 | 3.5 | 2.1 |
| Brain (substantia nigra) | 1.8 | 0.0 | 0.4 | 0.1 |
| Brain (thalamus) | 19.3 | 2.2 | 2.2 | 3.2 |
| Cerebral Cortex | 8.0 | 100.0 | 4.8 | 3.6 |
| Spinal cord | 1.4 | 1.1 | 0.4 | 1.0 |
| glio/astro U87-MG | 13.6 | 12.0 | 18.8 | 26.1 |
| glio/astro U-118-MG | 82.4 | 20.9 | 100.0 | 100.0 |
| astrocytoma SW1783 | 27.9 | 21.5 | 24.8 | 19.3 |
| neuro*; met SK-N-AS | 31.2 | 8.7 | 18.8 | 16.3 |
| astrocytoma SF-539 | 25.2 | 19.8 | 22.2 | 19.3 |
| astrocytoma SNB-75 | 20.6 | 5.2 | 27.2 | 15.7 |
| glioma SNB-19 | 4.7 | 1.6 | 4.0 | 3.4 |
| glioma U251 | 100.0 | 7.9 | 88.3 | 76.8 |
| glioma SF-295 | 5.6 | 3.3 | 5.6 | 3.5 |
| Heart (Fetal) | 1.0 | 4.3 | 0.3 | 0.3 |
| Heart | 0.7 | 0.3 | 0.0 | 0.0 |
| Skeletal muscle (Fetal) | 1.0 | 32.8 | 2.3 | 1.3 |
| Skeletal muscle | 6.0 | 2.0 | 0.0 | 0.2 |
| Bone marrow | 0.0 | 0.0 | 0.0 | 0.0 |
| Thymus | 0.2 | 0.7 | 0.5 | 0.6 |
| Spleen | 0.7 | 0.3 | 1.0 | 0.9 |
| Lymph node | 2.0 | 0.2 | 2.4 | 2.0 |
| Colorectal | 0.3 | 3.2 | 0.5 | 0.1 |
| Stomach | 3.4 | 0.1 | 2.2 | 0.1 |
| Small intestine | 3.5 | 0.6 | 1.3 | 0.7 |
| Colon ca. SW480 | 1.6 | 0.7 | 2.4 | 2.0 |
| Colon ca.* SW620 (SW480 met) | 0.0 | 0.0 | 0.0 | 0.0 |
| Colon ca. HT29 | 0.7 | 0.7 | 0.6 | 0.8 |
| Colon ca. HCT-116 | 0.3 | 0.0 | 0.0 | 0.1 |
| Colon ca. CaCo-2 | 8.6 | 14.3 | 9.7 | 7.4 |
| CC Well to Mod Diff (ODO3866) | 2.6 | 2.5 | 2.6 | 1.4 |
| Colon ca. HCC-2998 | 1.0 | 0.4 | 2.4 | 1.2 |
| Gastric ca. (liver met) NCI-N87 | 0.9 | 0.3 | 2.4 | 0.6 |
| Bladder | 0.9 | 2.5 | 2.3 | 0.4 |
| Trachea | 0.8 | 0.3 | 0.0 | 0.2 |
| Kidney | 0.8 | 0.5 | 0.0 | 0.0 |
| Kidney (fetal) | 2.8 | 1.4 | 2.5 | 1.3 |
| Renal ca. 786-0 | 11.2 | 6.4 | 19.9 | 9.5 |
| Renal ca. A498 | 13.1 | 4.3 | 13.2 | 7.2 |
| Renal ca. RXF 393 | 21.5 | 7.2 | 21.3 | 26.1 |
| Renal ca. ACHN | 10.1 | 5.1 | 7.6 | 7.5 |
| Renal ca. UO-31 | 10.2 | 3.3 | 13.8 | 9.5 |
| Renal ca. TK-10 | 0.0 | 0.0 | 0.0 | 0.0 |
| Liver | 0.0 | 0.0 | 0.0 | 0.0 |
| Liver (fetal) | 0.1 | 0.0 | 0.0 | 0.0 |
| Liver ca. (hepatoblast) HepG2 | 0.2 | 0.2 | 0.0 | 0.4 |
| Lung | 0.4 | 0.1 | 0.2 | 0.0 |
| Lung (fetal) | 0.3 | 0.3 | 0.0 | 0.7 |
| Lung ca. (small cell) LX-1 | 0.0 | 0.0 | 0.0 | 0.0 |
| Lung ca. (small cell) NCI-H69 | 3.1 | 11.6 | 5.4 | 11.2 |
| Lung ca. (s.cell var.) SHP-77 | 2.4 | 1.7 | 0.0 | 0.0 |
| Lung ca. (large cell)NCI-H460 | 18.6 | 2.6 | 26.1 | 12.9 |
| Lung ca. (non-sm. cell) A549 | 0.4 | 0.1 | 0.6 | 0.2 |
| Lung ca. (non-s.cell) NCI-H23 | 1.4 | 2.1 | 1.2 | 0.1 |
| Lung ca. (non-s.cell) HOP-62 | 9.5 | 3.9 | 16.0 | 6.8 |
| Lung ca. (non-s.cl) NCI-H522 | 28.1 | 36.9 | 15.3 | 5.8 |
| Lung ca. (squam.) SW 900 | 0.6 | 0.1 | 0.2 | 0.1 |
| Lung ca. (squam.) NCI-H596 | 16.5 | 8.0 | 19.2 | 12.3 |
| Mammary gland | 0.7 | 0.5 | 0.5 | 0.2 |
| Breast ca.* (pl.ef) MCF-7 | 5.0 | 8.8 | 5.1 | 2.1 |
| Breast ca.* (pl.ef) MDA-MB-231 | 2.4 | 0.3 | 0.5 | 0.4 |
| Breast ca.* (pl. ef) T47D | 53.6 | 26.1 | 1.9 | 1.1 |
| Breast ca. BT-549 | 0.0 | 0.0 | 0.0 | 0.0 |
| Breast ca. MDA-N | 0.8 | 1.1 | 1.5 | 1.1 |
| Ovary | 0.8 | 2.8 | 0.3 | 0.0 |
| Ovarian ca. OVCAR-3 | 58.6 | 19.3 | 26.8 | 20.0 |
| Ovarian ca. OVCAR-4 | 2.4 | 0.4 | 3.1 | 2.0 |
| Ovarian ca. OVCAR-5 | 0.0 | 0.0 | 0.0 | 0.0 |
| Ovarian ca. OVCAR-8 | 8.7 | 6.7 | 1.7 | 2.8 |
| Ovarian ca. IGROV-1 | 3.1 | 1.5 | 0.0 | 0.4 |
| Ovarian ca. (ascites) SK-OV-3 | 27.9 | 6.7 | 22.2 | 0.0 |
| Uterus | 2.4 | 0.4 | 1.2 | 0.9 |
| Placenta | 8.1 | 4.4 | 7.7 | 4.1 |
| Prostate | 2.1 | 0.1 | 0.0 | 0.0 |
| Prostate ca.* (bone met) PC-3 | 0.7 | 1.1 | 0.0 | 0.0 |
| Testis | 4.5 | 1.1 | 0.0 | 0.1 |
| Melanoma Hs688(A).T | 10.0 | 20.4 | 12.8 | 7.5 |
| Melanoma* (met) Hs688(B).T | 12.5 | 18.9 | 12.0 | 4.2 |
| Melanoma UACC-62 | 1.2 | 0.3 | 0.4 | 0.3 |
| Melanoma M14 | 13.7 | 2.1 | 14.4 | 7.8 |
| Melanoma LOX IMVI | 1.2 | 1.2 | 0.0 | 0.0 |
| Melanoma* (met) SK-MEL-5 | 3.7 | 4.5 | 3.8 | 1.8 |
| Adipose | 3.6 | 4.5 | 12.9 | 0.6 |

TABLE 10
Panel 2D
Column A - Rel. Exp.(%) Ag2674, Run 162455917
Column B - Rel. Exp.(%) Ag2820, Run 163578010
Column C - Rel. Exp.(%) Ag2820, Run 165910586

| Tissue Name | A | B | C | Tissue Name | A | B | C |
|---|---|---|---|---|---|---|---|
| Normal Colon | 47.6 | 12.4 | 15.7 | Kidney Margin 8120608 | 6.9 | 1.7 | 3.7 |
| CC Well to Mod Diff (ODO3866) | 8.4 | 7.2 | 7.4 | Kidney Cancer 8120613 | 0.5 | 0.0 | 0.0 |
| CC Margin (ODO3866) | 8.0 | 0.8 | 0.4 | Kidney Margin 8120614 | 2.8 | 1.6 | 0.0 |
| CC Gr.2 rectosigmoid | 5.4 | 3.8 | 2.3 | Kidney Cancer 9010320 | 22.4 | 39.5 | 36.1 |
| (ODO3868) | | | | Kidney Margin 9010321 | 14.1 | 22.5 | 11.6 |
| CC Margin (ODO3868) | 12.4 | 2.2 | 1.2 | Normal Uterus | 7.1 | 4.1 | 7.0 |
| CC Mod Diff (ODO3920) | 0.4 | 0.7 | 0.0 | Uterine Cancer 064011 | 38.4 | 5.5 | 2.3 |
| CC Margin (ODO3920) | 12.2 | 1.6 | 1.4 | Normal Thyroid | 13.9 | 4.7 | 1.1 |
| CC Gr.2 ascend colon | 3.8 | 2.9 | 3.6 | Thyroid Cancer | 30.4 | 36.3 | 40.9 |
| (ODO3921) | | | | Thyroid Cancer A302152 | 8.3 | 5.8 | 2.8 |
| CC Margin (ODO3921) | 8.9 | 1.3 | 0.0 | Thyroid Margin A302153 | 88.3 | 10.0 | 7.2 |
| CC from Partial Hepatectomy | 6.0 | 12.3 | 12.5 | Normal Breast | 26.4 | 9.5 | 11.3 |
| (ODO4309) Mets | | | | Breast Cancer | 2.0 | 0.7 | 0.8 |
| Liver Margin (ODO4309) | 0.4 | 0.4 | 0.0 | Breast Cancer (OD04590- | 13.7 | 4.0 | 2.9 |
| Colon mets to lung (OD04451- | 1.4 | 1.5 | 1.1 | 01) | | | |
| 01) | | | | Breast Cancer Mets | 55.1 | 32.5 | 15.9 |
| Lung Margin (OD04451-02) | 0.7 | 0.0 | 0.8 | (OD04590-03) | | | |
| Normal Prostate 6546-1 | 14.1 | 6.3 | 2.0 | Breast Cancer Metastasis | 24.8 | 12.2 | 2.9 |
| Prostate Cancer (OD04410) | 26.8 | 4.9 | 4.1 | Breast Cancer | 11.2 | 7.5 | 5.5 |
| Prostate Margin (OD04410) | 27.0 | 6.0 | 1.9 | Breast Cancer | 11.1 | 1.8 | 1.3 |
| Prostate Cancer (OD04720-01) | 18.8 | 3.2 | 1.2 | Breast Cancer 9100266 | 11.8 | 3.5 | 1.2 |
| Prostate Margin (OD04720-02) | 41.2 | 8.0 | 3.9 | Breast Margin 9100265 | 13.2 | 4.9 | 1.7 |
| Normal Lung | 16.0 | 13.4 | 11.8 | Breast Cancer A209073 | 19.2 | 3.5 | 1.7 |
| Lung Met to Muscle (ODO4286) | 25.5 | 64.2 | 39.2 | Breast Margin A209073 | 25.3 | 0.6 | 2.0 |
| Muscle Margin (ODO4286) | 14.1 | 1.3 | 1.1 | Normal Liver | 1.7 | 1.2 | 0.3 |
| Lung Malignant Cancer | 44.8 | 66.9 | 57.8 | Liver Cancer | 0.5 | 0.0 | 0.0 |
| (OD03126) | | | | Liver Cancer 1025 | 0.0 | 0.0 | 0.0 |
| Lung Margin (OD03126) | 11.7 | 10.6 | 5.9 | Liver Cancer 1026 | 0.7 | 0.0 | 0.0 |
| Lung Cancer (OD04404) | 13.7 | 10.4 | 11.6 | Liver Cancer 6004-T | 0.5 | 0.0 | 0.0 |
| Lung Margin (OD04404) | 11.4 | 10.7 | 14.4 | Liver Tissue 6004-N | 0.6 | 1.0 | 0.3 |
| Lung Cancer (OD04565) | 13.1 | 8.5 | 4.5 | Liver Cancer 6005-T | 1.1 | 0.0 | 0.0 |
| Lung Margin (OD04565) | 3.1 | 5.3 | 6.2 | Liver Tissue 6005-N | 0.0 | 0.0 | 0.0 |
| Lung Cancer (OD04237-01) | 7.4 | 13.6 | 4.5 | Normal Bladder | 26.1 | 14.7 | 12.7 |
| Lung Margin (OD04237-02) | 4.8 | 5.3 | 3.8 | Bladder Cancer | 6.0 | 9.2 | 2.0 |
| Ocular Mel Met to Liver | 0.9 | 0.0 | 0.0 | Bladder Cancer | 6.0 | 3.9 | 2.3 |
| (ODO4310) | | | | Bladder Cancer (OD04718- | 41.8 | 89.5 | 82.4 |
| Liver Margin (ODO4310) | 5.0 | 0.0 | 0.3 | 01) | | | |
| Melanoma Metastasis | 29.7 | 57.4 | 31.6 | Bladder Normal Adjacent | 22.4 | 3.5 | 3.9 |
| Lung Margin (OD04321) | 4.3 | 7.0 | 3.5 | (OD04718-03) | | | |
| Normal Kidney | 27.7 | 18.9 | 14.4 | Normal Ovary | 10.1 | 2.1 | 0.6 |
| Kidney Ca, Nuclear grade 2 | 2.9 | 5.6 | 2.9 | Ovarian Cancer | 100.0 | 36.3 | 100.0 |
| (OD04338) | | | | Ovarian Cancer (OD04768- | 0.3 | 0.0 | 0.4 |
| Kidney Margin (OD04338) | 11.8 | 10.8 | 9.0 | 07) | | | |
| Kidney Ca Nuclear grade ½ | 48.3 | 82.4 | 67.8 | Ovary Margin (OD04768-08) | 8.2 | 6.9 | 4.4 |
| (OD04339) | | | | Normal Stomach | 5.7 | 2.2 | 1.9 |
| Kidney Margin (OD04339) | 15.9 | 17.7 | 8.8 | Gastric Cancer 9060358 | 7.2 | 3.0 | 2.8 |
| Kidney Ca, Clear cell type | 0.8 | 0.0 | 0.3 | Stomach Margin 9060359 | 4.9 | 0.7 | 1.5 |
| (OD04340) | | | | Gastric Cancer 9060395 | 6.5 | 1.9 | 1.8 |
| Kidney Margin (OD04340) | 21.6 | 13.9 | 8.0 | Stomach Margin 9060394 | 7.2 | 2.2 | 2.3 |
| Kidney Ca, Nuclear grade 3 | 33.4 | 84.7 | 58.2 | Gastric Cancer 9060397 | 46.7 | 22.7 | 28.5 |
| (OD04348) | | | | Stomach Margin 9060396 | 4.7 | 0.7 | 0.0 |
| Kidney Margin (OD04348) | 12.9 | 4.6 | 11.1 | Gastric Cancer 064005 | 5.6 | 9.2 | 6.5 |
| Kidney Cancer (OD04622-01) | 1.4 | 0.0 | 4.6 | | | | |
| Kidney Margin (OD04622-03) | 7.3 | 3.9 | 1.1 | | | | |
| Kidney Cancer (OD04450-01) | 84.7 | 100.0 | 78.5 | | | | |
| Kidney Margin (OD04450-03) | 19.9 | 12.0 | 6.9 | | | | |
| Kidney Cancer 8120607 | 12.7 | 4.9 | 4.2 | | | | |

TABLE 11
Panel 4D
Column A - Rel. Exp.(%) Ag1479, Run 162599612
Column B - Rel. Exp.(%) Ag2674, Run 160645450
Column C - Rel. Exp.(%) Ag2820, Run 162350531
Column D - Rel. Exp.(%) Ag2820, Run 164329602

| Tissue Name | A | B | C | D |
|---|---|---|---|---|
| Secondary Th1 act | 0.3 | 0.0 | 0.0 | 0.0 |
| Secondary Th2 act | 0.0 | 0.0 | 0.0 | 0.5 |
| Secondary Tr1 act | 0.0 | 0.0 | 0.0 | 0.3 |
| Secondary Th1 rest | 0.0 | 0.0 | 0.0 | 0.0 |
| Secondary Th2 rest | 0.0 | 0.0 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.0 | 0.0 | 0.0 |
| Primary Tr1 act | 0.0 | 0.0 | 0.0 | 0.0 |
| Primary Th1 rest | 0.0 | 0.5 | 0.0 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 lymphocyte act | 1.8 | 1.0 | 1.6 | 0.8 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 lymphocyte rest | 0.0 | 0.0 | 0.0 | 0.0 |
| Secondary CD8 lymphocyte act | 0.0 | 0.0 | 0.0 | 0.0 |
| CD4 lymphocyte none | 0.0 | 0.0 | 0.0 | 0.0 |
| 2ry Th1/Th2/Tr1 anti-CD95 CH11 | 0.0 | 0.0 | 0.0 | 0.0 |
| LAK cells rest | 0.0 | 0.0 | 0.0 | 0.0 |
| LAK cells IL-2 | 0.0 | 0.0 | 0.3 | 0.0 |
| LAK cells IL-2 + IL-12 | 0.0 | 0.0 | 0.0 | 0.7 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 0.0 | 0.0 | 0.0 | 0.5 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 0.0 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 0.0 | 0.0 | 0.3 | 2.5 |
| B lymphocytes CD40L and IL-4 | 0.2 | 0.4 | 0.0 | 0.0 |
| EOL-1 dbcAMP | 0.2 | 0.2 | 0.3 | 0.7 |
| EOL-1 dbcAMP PMA/ionomycin | 0.1 | 0.2 | 0.9 | 0.0 |
| Dendritic cells none | 0.0 | 0.0 | 0.0 | 0.0 |
| Dendritic cells LPS | 0.0 | 0.0 | 0.0 | 0.0 |
| Dendnitic cells anti-CD40 | 0.0 | 0.0 | 0.0 | 0.0 |
| Monocytes rest | 0.0 | 0.0 | 0.0 | 0.0 |
| Monocytes LPS | 0.0 | 0.0 | 0.0 | 0.0 |
| Macrophages rest | 0.0 | 0.0 | 0.0 | 0.0 |
| Macrophages LPS | 0.0 | 0.0 | 0.0 | 0.0 |
| HUVEC none | 23.0 | 17.7 | 0.0 | 0.0 |
| HUVEC starved | 25.0 | 26.1 | 0.0 | 0.0 |
| HUVEC IL-1beta | 8.1 | 7.1 | 0.0 | 0.0 |
| HUVEC IFN gamma | 14.8 | 13.8 | 0.0 | 0.3 |
| HUVEC TNF alpha + IFN gamma | 8.1 | 6.7 | 0.0 | 0.0 |
| HUVEC TNF alpha + IL4 | 12.0 | 10.2 | 0.0 | 0.0 |
| HUVEC IL-11 | 8.5 | 7.0 | 0.0 | 0.0 |
| Lung Microvascular EC none | 11.1 | 14.2 | 0.0 | 0.0 |
| Lung Microvascular EC TNF alpha + | 9.3 | 11.0 | 0.0 | 0.2 |
| IL-1beta | | | | |
| Microvascular Dermal EC none | 100.0 | 75.3 | 0.0 | 0.0 |
| Microsvasular Dermal EC TNF alpha + | 29.7 | 26.8 | 0.0 | 0.0 |
| IL-1beta | | | | |
| Bronchial epithelium TNF alpha + IL1beta | 0.2 | 1.3 | 2.4 | 19.9 |
| Small airway epithelium none | 2.2 | 1.1 | 1.0 | 1.7 |
| Small airway epithelium TNF alpha + | 0.3 | 0.2 | 0.0 | 0.0 |
| IL-1beta | | | | |
| Coronery artery SMC rest | 8.3 | 8.0 | 1.9 | 2.6 |
| Coronery artery SMC TNF alpha + | 4.6 | 3.1 | 3.0 | 1.2 |
| IL-1beta | | | | |
| Astrocytes rest | 85.9 | 70.2 | 100.0 | 100.0 |
| Astrocytes TNF alpha + IL-1beta | 59.0 | 100.0 | 71.7 | 65.5 |
| KU-812 (Basophil) rest | 0.0 | 0.3 | 0.0 | 0.0 |
| KU-812 (Basophil) PMA/ionomycin | 0.0 | 0.0 | 0.0 | 0.0 |
| CCD1106 (Keratinocytes) none | 19.8 | 17.2 | 35.6 | 70.2 |
| CCD1106 (Keratinocytes) TNF alpha + | 1.7 | 1.3 | 13.4 | 29.3 |
| IL-1beta | | | | |
| Liver cirrhosis | 0.0 | 0.5 | 0.3 | 0.0 |
| Lupus kidney | 1.8 | 2.9 | 6.2 | 8.1 |
| NCI-H292 none | 0.0 | 0.0 | 0.0 | 0.0 |
| NCI-H292 IL-4 | 0.0 | 0.0 | 0.3 | 0.4 |
| NCI-H292 IL-9 | 0.0 | 0.0 | 0.0 | 0.0 |
| NCI-H292 IL-13 | 0.0 | 0.0 | 0.0 | 0.0 |
| NCI-H292 IFN gamma | 0.0 | 0.0 | 0.0 | 0.0 |
| HPAEC none | 15.1 | 12.2 | 0.0 | 0.0 |
| HPAEC TNF alpha + IL-1beta | 6.2 | 7.5 | 0.6 | 0.0 |
| Lung fibroblast none | 0.9 | 0.4 | 0.0 | 0.4 |
| Lung fibroblast TNF alpha + IL-1beta | 0.6 | 0.0 | 0.0 | 0.0 |
| Lung fibroblast IL-4 | 2.1 | 2.9 | 1.7 | 3.7 |
| Lung fibroblast IL-9 | 1.2 | 0.5 | 1.2 | 2.0 |
| Lung fibroblast IL-13 | 1.2 | 0.9 | 1.6 | 3.3 |
| Lung fibroblast IFN gamma | 2.1 | 1.9 | 2.3 | 0.2 |
| Dermal fibroblast CCD1070 rest | 10.5 | 9.8 | 10.3 | 8.4 |
| Dermal fibroblast CCD1070 TNF alpha | 11.6 | 4.6 | 10.0 | 11.3 |
| Dermal fibroblast CCD1070 IL-1beta | 4.9 | 2.2 | 4.5 | 3.8 |
| Dermal fibroblast IFN gamma | 1.2 | 1.7 | 0.3 | 1.6 |
| Dermal fibroblast IL-4 | 28.3 | 27.9 | 12.1 | 13.4 |
| IBD Colitis 2 | 0.7 | 1.6 | 0.3 | 0.0 |
| IBD Crohn's | 1.6 | 0.4 | 0.8 | 3.7 |
| Colon | 8.6 | 7.6 | 1.7 | 1.9 |
| Lung | 2.0 | 2.9 | 3.8 | 6.3 |
| Thymus | 7.0 | 13.7 | 4.1 | 4.4 |
| Kidney | 17.0 | 27.5 | 13.0 | 20.2 |

Panel 1.3D Summary: Ag2820

The expression of the CG53018-01 gene was assessed in two independent runs in panel 1.3D, with good concordance between the different runs. Overall, the expression of this gene is highest in brain cancer cell lines. In addition, there is substantial expression in other samples derived from cancer cell lines, such as lung cancer, and ovarian cancer. Thus, the expression of this gene could be used to distinguish these samples from other samples in the panel. Moreover, therapeutic modulation of this gene, through the use of small molecule drugs, antibodies or protein therapeutics is of use in the treatment of brain cancer, lung cancer, or ovarian cancer.

Panel 2D Summary: Ag690/Ag2820

The expression of the CG53018-01 gene was assessed in three independent runs in panel 2D using two different probe/primer sets. The highest expression of this gene is generally associated with kidney cancers. Of particular note is the consistent absence of expression in normal kidney tissue adjacent to malignant kidney. In addition, there is substantial expression associated with ovarian cancer, bladder cancer and lung cancer. This is consistent with the expression seen in Panel 1.3D. Thus, the expression of this gene could be used to distinguish the above listed malignant tissue from other tissues in the panel. Particularly, the expression of this gene could be used to distinguish malignant kidney tissue from normal kidney. Moreover, therapeutic modulation of this gene, through the use of small molecule drugs, antibodies or protein therapeutics is of benefit in the treatment of kidney cancer, ovarian cancer, bladder cancer or lung cancer.

Panel 4D Summary: Ag018b/Ag2820

Two out of three experiments show highest expression of the CG53018-01 transcript is highest in astrocytes and microvascular dermal endothelial cells (CTs=29-30), with low but significant expression in keratinocytes, and dermal fibroblasts. Expression is not modulated by any treatment, suggesting that this protein may be important in normal homeostasis. Thus, this transcript or the protein it encodes could be used to identify the tissues and cells in which it is expressed.

### Example 4

### Molecular Cloning of the Extracellular Domain of CG55069

The open reading frame of CG55069-04 codes for an extracellular domain of Ten-M3. Oligonucleotide primers were designed to PCR amplify a DNA segment, representing an ORF, coding for CG55069-04. The forward primer includes, a Hind III restriction site while the reverse primer contains an, in frame, Sal I restriction site for further subcloning purposes.

The open reading frame of CG55069-11 codes for an extracellular domain of Ten-M3. Oligonucleotide primers were designed to PCR amplify a DNA segment, representing an ORF, coding for CG55069-11. The forward primer includes, a NruI restriction site while the reverse primer contains an, in frame, XhoI restriction site for further subcloning purposes.

PCR reactions using the specific primers for CG55069-04 and CG55069-11 were set up using a total of 5 ng cDNA template containing equal parts of cDNA samples derived from human adrenal gland, human testis, human mammary, human skeletal muscle, and fetal brain; 1 μM of each of the Sem6A FORW and Sem6A FL-REV primers, 5 micromoles dNTP (Clontech Laboratories, Palo Alto Calif.) and 1 μl of 50×Advantage-HF 2 polymerase (Clontech Laboratories, Palo Alto Calif.) in 50 μl volume. An approximately 1 kbp large amplified product was isolated from agarose gel and ligated to pCR2.1 vector (Invitrogen, Carlsbad, Calif.). The cloned insert was sequenced, using vector specific, M13 Forward (−40) and M13 Reverse primers and verified as an open reading frame coding for CG55069-04 and CG55069-11. The EGF domain of Ten-M3 is thought to mediate dimerization of the protein, which may be necessary for proper functioning of the protein. CG55069-04 (SEQ ID NO: 12) and CG55069-11 (SEQ ID NO: 6) were tagged with V5 and 6×His, providing CG55069-18 and CG55069-19 respectively. Addition of tags to CG55069-04 and CG55069-11 resulted in the following:
CG55069-18 at the N-terminus has DAAQPARRARRTKL (SEQ ID NO:36) which accounts for amino acid 1 to 14 of SEQ ID NO: 18 wherein D is the remaining product of cleaved IgK signal sequence and MQPARRARRTKL (SEQ ID NO:37) is the filler sequence from vector (linkers, MCS etc.). At the C-terminus, CG55069-18 has LEGKPIPNPLLGLDSTRTGHHHHHH (SEQ ID NO:38) which accounts for amino acid 282-296 of SEQ ID NO: 18, wherein LE is a filler sequence from the vector, GKPIPNPLLGLDST (SEQ ID NO:38) is the V5 tag, RTG is a filler followed by 6×His tag.
CG55069-19 at the N-terminus has DAAQPARRARRTKLSR (SEQ ID NO:36) which accounts for amino acid 1 to 16 of SEQ ID NO: 20 wherein D is the remaining product of cleaved IgK signal sequence and AAQPARRARRTKLSR (SEQ ID NO:37) is the filler sequence from vector (linkers, MCS etc.).
At the C-terminus, CG55069-19 has LEGKPIPNPLLGLDSTRTGHHHHHH (SEQ ID NO:38) which accounts for amino acid 838-862of SEQ ID NO: 38, wherein LE is a filler sequence from the vector, GKPIPNPLLGLDST (SEQ ID NO:38) is the V5 tag, RTG is a filler followed by 6×His tag.

### Example 3

### Expression of CG55069 in Human Embryonic Kidney 293 Cells

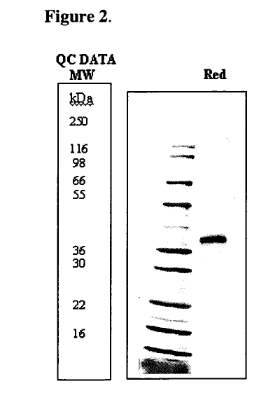

CG55069-04 was subcloned into expression vector pCEP-sec vector, generated Plasmid 1266. The resulting plasmid 1266 was transfected into 293 cells using the LipofectaminePlus reagent following the manufacturer's instructions (Gibco/BRL). The cell pellet and supernatant were harvested 72 h post transfection and examined for CG55069-04 expression by Western blot (reducing conditions) using an anti-V5 antibody. It was expressed in the mammalian expression system and purified to homogeneity (FIG. 2).

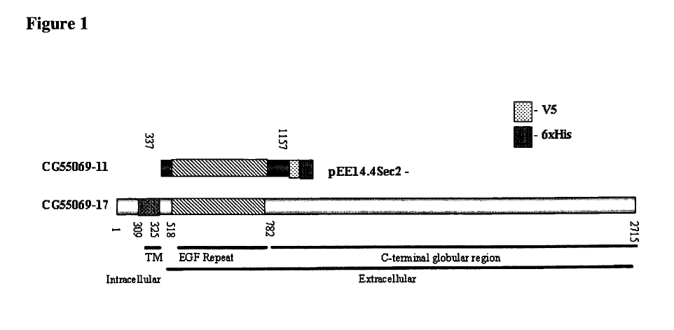

CG55069-11 was subcloned into expression vector pEE14.4Sec2 vector, generated Plasmid 2735 (FIG. 1). The resulting plasmid 2735 was transfected into 293 cells using the LipofectaminePlus reagent following the manufacturer's instructions (Gibco/BRL). The cell pellet and supernatant were harvested 72 h post transfection and examined for CG55069-11 expression by Western blot (reducing conditions) using an anti-V5 antibody. It was expressed in the mammalian expression system and purified to homogeneity.

### Example 4

### CG55069 Inhibition of Cell Migration

CG55069 was expressed in a number of tissues, including vascularized tissues. The mRNA expression profile of CG55069 (Example 1) was striking in that it was elevated in renal and lung tumor tissues as well as in HUVEC and in a majority of renal clear cell carcinoma (RCC) cell lines, suggesting that CG55069 plays a role in endothelial cell processes and potentially tumor neovascularization. Migration of endothelial cells is one of the important processes in the angiogenic cascade. Thus role of CG55069 polypeptide in the migration process was tested as described below.

To determine if Ten-M3 proteins influence cell migration, cell lines were screened for cell motility in response to various treatments. Cell lines tested include: HUVEC (human umbilical vein endothelial cells), HMVEC-d (human microvascular endothelial cells), 786-0 (renal carcinoma, epithelial), and H1299 (p53-null lung cancer cell line). 24-well transwell (BD Biosciences, Bedford, Mass.) migration chambers (8 μm pore size) were used. Briefly, 4×10⁴ cells in serum free medium (Medium 200 for HUVEC, Medium 131 for HMVEC-d, and DMEM high glucose/1% Penicillin/Streptomycin/10% FBS for the cancer cell lines) containing 0.1% BSA were added to wells in the upper chamber (300 μl). The chambers were pre-coated with Type I Collagen at 10 μg/ml for 1 h at 37° C. The lower chamber was filled with chemotactant (1% FBS supplemented with 10 ng/ml of VEGF). CG55069-04 or CG55069-11 in various concentrations ranging from 1 ng/ml to 100 ng/ml was added to the upper chamber and the cells were incubated at 37° C. Following incubation, cells on the upper surface of the membrane (non-migrated cells) were scraped with a cotton swab. Cells on the lower side of the membrane (migrated cells) were stained with 0.2% Crystal Violet dye (Fisher Scientific, Springfield, N.J.) in 70% ethanol for 30 min. The cells were then de-stained in PBS, pH 7.4 and the membrane was left to air dry at room temperature. Migrated cells were counted using a Zeiss Axiovert 100 inverted microscope. Three independent areas per filter were counted and the mean number of migrated cells was calculated. An RGD control peptide (Invitrogen; Cat. No. 12135-018) with the amino acid sequence “GRGDSP” was used as a positive control for the endothelial cell lines, and fetal bovine serum (FBS) ranging from 0.5% to 2% (with or without VEGF, depending on the cell line) was used as a positive control for the cancer cell lines. Serum free media (SFM) was used as a negative control.

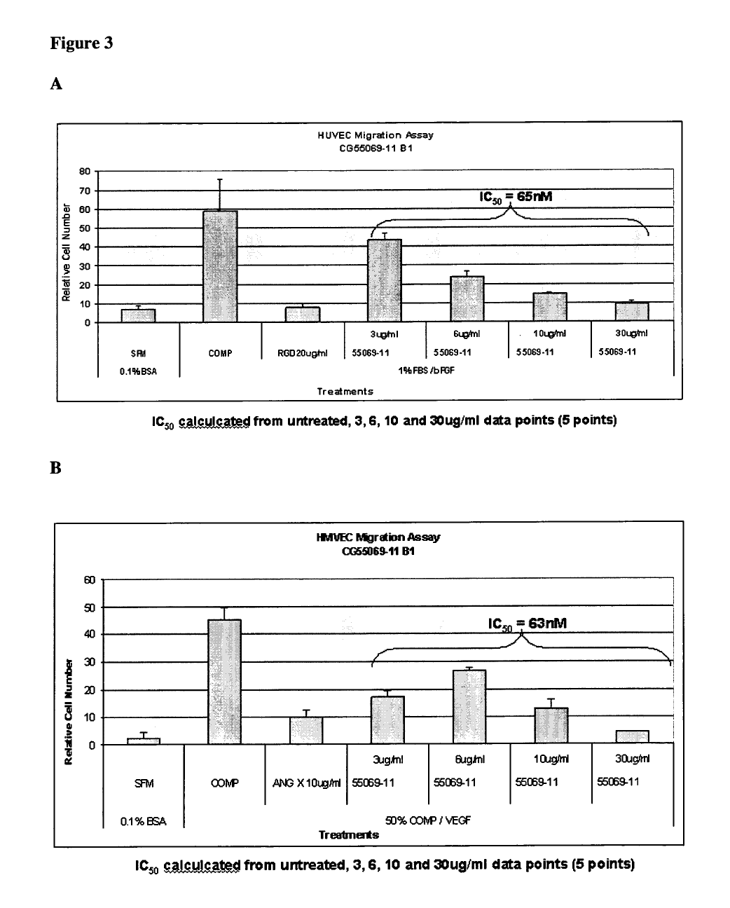

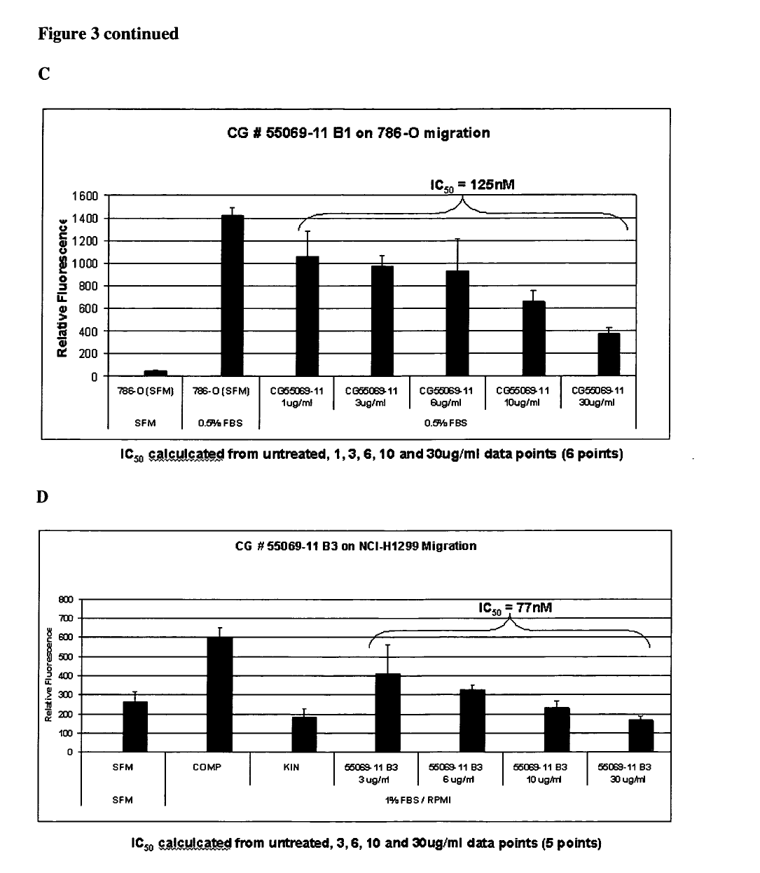

Ten-M3 variants CG55069-04 and CG55069-11 significantly inhibited the VEGF-induced migration of endothelial cells in a dose-dependent manner. The inhibition of migration was seen in human umbilical vein (HUVEC) (FIG. 3A) as well as microvascular endothelial cells (HMVEC-d) (FIG. 3B). CG55069-11 inhibited the migration of both 786-0 and H1299 lung cancer cells (FIGS. 3C and 3D). These data indicate that the EGF domain of Ten-M3 (CG55069-04 and CG55069-11) interferes with the described ability of these proteins to enhance cell migration. CG55069 proteins therefore demonstrate antiangiogenic and antimetastatic activity.

### Example 5

### In Vivo Activity of CG55069

CG55069 was tested for ability to inhibit neovascularization in vivo using a matrigel plug assays. Mice (nu/nu) were injected with 0.5 ml Matrigel prepared with 10 ng/mL of bFGF and 100 ng/mL of VEGFm, or 786-0 renal carcinoma cells. CG55069-19 was administered subcutaneously for 7 days. On day 7 all animals were euthanized and the Matrigel plugs excised and formalin-fixed. Three sections, 5 to 7 μm in thickness were cut from each Matrigel plug and were stained with hematoxylin and eosin. Sections were examined under phase contrast microscope. Representative photomicrographs were recorded [two frames (100× and 400×)]. Infiltration of endothelial cells and vessels were recorded.

Vessel staining by immunohistochemistry was done using the following protocol: Matrigel plugs sections were blocked with BSA (0.1%) and then treated with monoclonal antibody reactive to mouse CD31 conjugated to Phycoerythin (dilutions as recommended by the manufacturer). After thorough washing, sections were mounted with Vecta Shield and observed under UV microscope using a red filter. Representative digital images were captured (two images at 100× and 200× magnification). Morphometric analysis of vessel density was analyzed by immunofluorescence images of CD31 staining using the Skeletinization program. Data were processed to provide mean vessel density, node and length for each group.

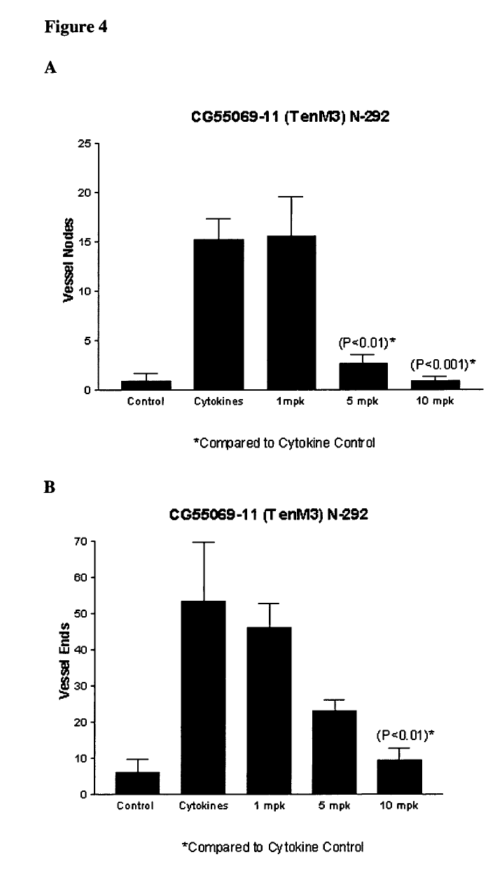

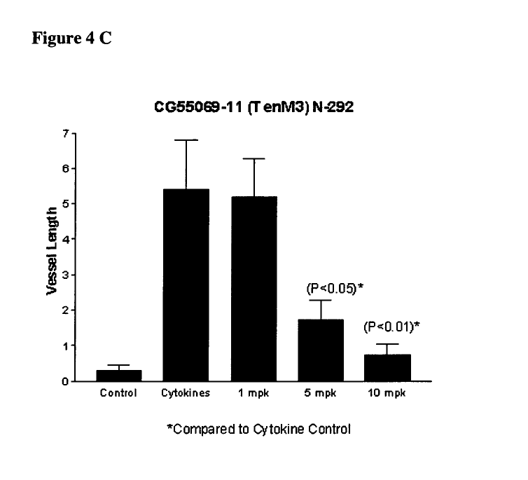

The effects of CG55069-19 on 786-0, renal carcinoma cell-induced angiogenesis in a matrigel plug assay were shown in FIG. 4. Gross morphology of the matrigel plugs indicate that CG55069 inhibited 786-0 renal carcinoma induced angiogenesis in athymic nude mice. When CG55069-19 was administered to mice, carcinoma cell induced vascularization was inhibited significantly. FIG. 4A shows the comparative angiogenic response in terms of the number of vessel nodes generated in the control group (matrigel alone) 1.0 compared to 17 nodes per unit area in 786-0 cell matrigel plugs and 3 or 1 in CG55069 treated (5 mpk and 10 mpk repectively) specimens. Treatment with CG55069-19 also significantly reduced the number of vessel ends recorded in specimens (FIG. 4B). CG55069 treatment also resulted in showed marked inhibition in total vessel length detected in specimens as compared to controls (FIG. 4C).

### Example 6

### CG55069 Inhibition of Human Tumor Xenograft

Athymic nude mice (nu/nu) are implanted with either tumor cells or tumor fragments from an existing host. After the implanted tumor reaches a volume of 100 mm³, animals are randomized into treatment groups. CG55069 is administered via conventional routes (IP, SQ, IV or IM) for a period of 2 weeks. Daily individual animal weights are recorded through the dosing period and twice weekly thereafter. Twice weekly, tumor size is determined. Tumor volume is determined using the formula: Tumor volume (in mm³)=(length×width×height)×0.536. The volume determinations for the treated groups is compared to the untreated tumor bearing control group. The difference in time for the treated tumors to reach specific volumes (500, 1000, 1500 and 2000 mm³) is calculated. It is demonstrated that CG55069 treatment causes a delay in the growth of tumors in vivo.

### Example 7

### CG55069 Binding to Cells Demonstrated by Flow Cytometry

Flow cytometry (FACs) analysis was performed to demonstrate Ten-M3 binding to the cell membrane of specific cells. FACs analysis was performed on cell lines determined to have increased CG55069 expression by RTQ-PCR, including 786-0, U87, and HUVEC cells. Cells were trypsinized, washed in complete media, resuspended in 1 ml 0.1% BSA/PBS solution, incubated with 30 μg/ml CG55069-11 on ice for 1 hr. Cells were then washed with 0.1% BSA/PBS. The ability of CG55069 to compete for heparin binding was determined in a competition experiment in which cells were subsequently incubated with different concentrations of heparin sulfate (Invitrogen) on ice for 1 hr. Cells were washed with 0.1% BSA/PBS and incubated with a 1:1000 dilution of Anti-V5 antibody (Invitrogen) on ice for 1 hr. After washing with 0.1% BSA/PBS, cells were incubated with a 1:200 dilution of Rabbit Anti-Mouse Phycoerythrin conjugate on ice for 1 hr, followed by 0.1% BSA/PBS wash. Cells were analyzed for PE fluorescence on the FL channel by FACS.

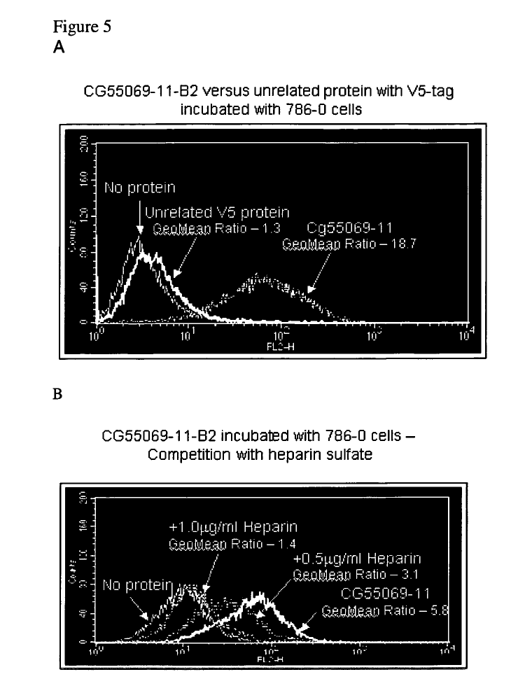

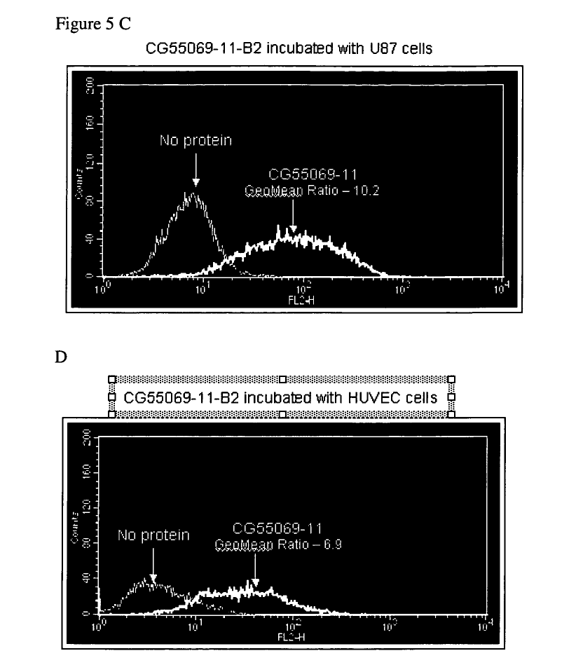

Binding of CG55069-11 to 786-0 cells was specific (FIG. 5A) and could be competed by heparin sulfalte (FIG. 5B). As heparin sulfalte is proteoglycan that acts as a cofactor in receptor signaling, these results indicate CG55069-11 acts as a dominant negative version of these factors. CG55069-11 also specifically bound U87 cells (FIG. 5C) and HUVEC cells (FIG. 5D).

### Example 8

### CG55069 Targeted Cell Killing

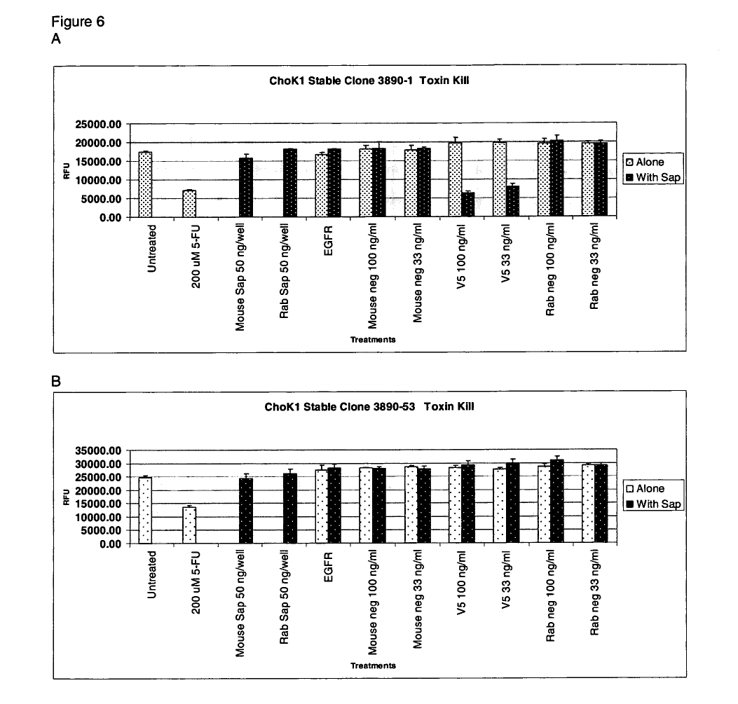

Targeted killing of cells expressing CG55069 was demonstrated using a primary antibody in combination with a toxin-conjugated secondary antibody reagent. The secondary reagent utilizes the toxin saporin at a concentration that requires the reagent be internalization to induce cell death. CHOK1 cells (clone 3890-53) and transfected CHOK1 cells expressing CG55069 (clone 3890-1) were plated at 2000 cells/well in 100 μl complete media in 96 well flat bottom tissue culture plates and incubates at 37° C. Cells were incubated with one of the following treatments: mouse secondary toxin 50 ng/well, rabbit secondary toxin 50 ng/well, V5 antibody in growth media 100 ng/ml, mouse neg antibody 100 ng/ml, rabbit neg antibody 100 ng/ml, EGFR positive control, 5-FU, untreated wells get 50 μl complete medi. Plates were tapped gently and incubatored. On Day 5 20 μl Cell Titer Blue was added to all wells and plates were read at 530 exc/580 em after 2.5 hours. FIG. 6 shows cells expressing CG55069 (FIG. 6A) compared to cells that did not express CG55069 (FIG. 6B) were killed by specific antibody saporin conjugate.

### Example 9

### Antibody to CG55069

Rabbit anti-CG55069 polyclonal antibodies (pAb) were generated using CG55069-11 as an immunogen, using methods known in the art. Binding of CG55069 pAb to various cell lines was assessed by FACs analysis. Adherant Cells were washed twice with PBS (Ca and Mg free), incubated with Versene at 37° C. until cells detached, counted and aliquoted at 1 million cells per assay tube. Cells were then washed twice and resuspended in ice-cold FACS buffer (0.01M HEPES, 0.15M NaCl, 0.1% NaN₃ and 4% FBS). Lymphoma or leukemia derived cells were washed twice with ice-cold FACS buffer and resuspended at 1 million cells per assay tube. Rabbit anti-CG55069 pAb was added to the cells. Cells were incubated on ice for 30 min, washed 2-3 times and resuspended in I ml of ice-cold FACS buffer. R-PE-conjugated goat anti-rabbit antibody (Jackson ImmunoResearch Laboratory) at 1:100 dilution was added and cells were incubated on ice for 30 min. After washing 3 times with 1 ml of ice-cold FACS buffer, cells were fixed with 0.5-1 ml of 1% formaldehyde in PBS and analyzed by Flow Cytometry.

Results showed that rabbit anti-CG55069 pAb positively stained OVCAR3 and MCF7 cells, weakly stained NCl-H69 and CAPAN 2 cells and was negative on NCl-H23 cells. Results of rabbit anti-CG55069 pAb staining of lymphoma and leukemia cells is shown in Table 12.

TABLE 12
Geo Mean Ratio of anti-CG55069 Staining
of Lymphoma and Leukemia Cells

| | Geo Mean Ratio | | |
|---|---|---|---|
| | | Rabbit | Rabbit anti- |
| Cell line | untreated | negative | CG55069 pAb |
| SR (anaplastic large | 3.51 | 4.29 | 6.28 |
| T cell lymphoma, ALCL) | | | |
| MOLT-4 (acute T cell | 3.27 | 4.13 | 3.58 |
| lymphoblastic leukemia) | | | |
| MV4-11 (myelomonocytic | 5.91 | 90.74 | 128.09 |
| leukemia) | | | |
| CCRF-CEM (acute T cell | 3.11 | 4.22 | 4.19 |
| lymphoblastic leukemia) | | | |
| Karpas 299 (ALCL) | 4.26 | 5.08 | 8.60 |
| SU-DHL-4 (B cell lymphoma) | 1.12 | 2.38 | 4.29 |
| SUP-M2 (ALCL) | 4.33 | 6.13 | 11.77 |
| DEL (ALCL) | 3.91 | 7.41 | 15.42 |

Thus we have illustrated and described the preferred embodiment of our invention, it is to be understood that this invention is capable of variation and modification, and we therefore do not wish to be limited to the precise terms set forth, but desire to avail ourselves of such changes and alterations which may be made for adapting the invention t various usages and conditions. Such alterations and changes may include, for different compositions for the administration of the polypeptides according to the present invention to a mammal; different amounts of the polypeptide; different times and means of administration; different materials contained in the administration dose including, for example combinations of different peptides, or combinations of peptides with different biologically active compounds. Such changes and alterations also are intended to include modifications in the amino acid sequence of the specific polypeptides described herein in which such changes alter the sequence in a manner as not to change the functionality of the polypeptide, but as to change solubility of the peptide in the composition to be administered to the mammal, absorption of the peptide by the body, protection of the polypeptide for either shelf life or within the body until such time as the biological action of the peptide is able to bring about the desired effect, and such similar modifications. Accordingly, such changes and alterations are properly intended to be within the full range of equivalents, and therefore within the purview of the following claims. Having thus described our invention and the manner and process of making and using it in such full, clear, concise and exact terms so as to enable any person skilled in the art to which it pertains, or with which it is most nearly connected, to make and use the same.

1. An isolated polypeptide comprising an amino acid sequence selected from the group consisting of SEQ ID NO: 2, 4, 6, 8, 10, 12, 14, 16, 18 and 20.

2. A composition comprising the polypeptide of claim 1 and a pharmaceutically acceptable carrier.

3. A kit comprising, in one more containers the composition of claim 2.

4. A method of modulating cell migration comprising contacting the cells with a polypeptide of claim 1.

5. The method of claim 4 wherein the cell is selected from the group consisting of an endothelial cell, an epithelial cell, a neuronal cell, a mesenchymal cell or a fibroblast cell.

6. The method of claim 5 wherein the endothelial cell is a microvascular endothelial cell or an umbilical vein endothelial cell.

7. The method of claim 4 wherein the cell is a cancer cell.

8. The method of claim 7 wherein the cancer cell is a renal cell carcinoma, a lung cancer cell or a pancreatic cancer cell.

9. A method of preventing or inhibiting angiogenesis or neovascularization in a mammal comprising administering a therapeutically effective amount of the polypeptide of claim 1 alone or together with a pharmaceutical carrier.

10. The method of claim 9 wherein the mammal is human.

11. An isolated polynucleotide comprising a nucleic acid sequence encoding the amino acid sequence selected from the group consisting of SEQ ID NO:2, 4, 6, 8, 10, 12, 14, 16, 18 and 20.

12. A vector comprising the polynucleotide of claim 11.

13. The vector of claim 12, wherein a promoter is operably linked to the said polynucleotide.

14. An isolated cell comprising the vector of claim 13.

15. The polynucleotide of claim 11 comprising a nucleic acid sequence selected from the group consisting of SEQ ID NO:1, 3, 5, 7, 9, 11, 13, 15, 17 and 19.

16. An isolated antibody that immunospecifically binds to the polypeptide of claim 1.

17. A method of modulating cell migration comprising contacting the cells with the antibody of claim 16.

18. The method of claim 17, wherein the cell is selected from the group consisting of an endothelial cell, an epithelial cell, a neuronal cell, a mesenchymal cell or a fibroblast cell.

19. The method of claim 17, wherein the cell is a cancer cell.

20. The method of claim 19, wherein the cancer cell is a renal cell carcinoma, a lung cancer cell, a breast cancer cell, an ovarian cancer cell or a pancreatic cancer cell.
